(19) United States
(12) Patent Application Publication
(10) Pub. No.: US 2026/0284066 A1
(43) Pub. Date: Sep. 24, 2026

(54) TYK2 INHIBITORS AND COMPOSITIONS AND METHODS THEREOF

(71) Applicant: LYNK PHARMACEUTICALS CO. LTD., Hangzhou (CN)

(72) Inventors: Xiaodong Li, Hangzhou (CN); Lin Su, Hangzhou (CN); Zhaokui Wan, Hangzhou (CN); Gurmit Grewal, Lexlington, MA (US); Michael Lawrence Vazquez, Creve Coeur, MO (US); Weiyu Lin, Hangzhou (CN)

(21) Appl. No.: 19/491,328

(22) PCT Filed: Jun. 14, 2024
(86) PCT No.: PCT/CN2024/099407

§ 371 Date: Dec. 8, 2025

(30) Foreign Application Priority Data
Jun. 15, 2023 (WO) PCT/CN2023/100508

(51) Int. Cl. A61K 31/5377; A61K 31/4439; A61K 31/444; A61K 31/501; A61K 31/506; A61K 31/519; A61K 31/695; A61P 29/00; A61P 35/00; C07B 59/00; C07D 401/12; C07D 401/14; C07D 471/04; C07D 487/04; C07F 7/08

(52) U.S. Cl. CPC A61K 31/5377; A61K 31/4439; A61K 31/444; A61K 31/501; A61K 31/506; A61K 31/519; A61K 31/695; A61P 29/00; A61P 35/00; C07B 59/002; C07B 59/004; C07D 401/12; C07D 401/14; C07D 471/04; C07D 487/04; C07F 7/0812

(57) ABSTRACT

The invention provides a novel class of therapeutic agents that are safe and effective TYK2 inhibitors and pharmaceutical compositions of these compounds and methods of preparation and use thereof against various TYK2-mediated diseases and disorders.

### PRIORITY CLAIMS AND RELATED PATENT APPLICATIONS

This application claims the benefit of priority to PCT International application No. PCT/CN2023/100508, filed Jun. 15, 2023, the entire content of which is incorporated herein by reference for all purposes.

### TECHNICAL FIELDS OF THE INVENTION

The invention generally relates to novel compounds and methods for their therapeutic use. More particularly, the invention provides a novel class of tyrosine kinase 2 inhibitors as well as pharmaceutical compositions of these compounds and methods of preparation and use thereof against various diseases and conditions.

### BACKGROUND OF THE INVENTION

Janus kinase (JAK) is a family of intracellular, nonreceptor tyrosine kinases that transduce cytokine-mediated signals via the Janus kinase—Signal Transduction Activators of Transcription (JAK-STAT) pathway. There are four members in the JAK family of enzymes in humans, i.e., JAK1, JAK2, JAK3 and tyrosine kinase 2 (TYK2). The family is defined by the presence of two adjacent kinase domains, JH1 and JH2, of which JH1 performs the phosphorylation involved in pathway activation whereas JH2 regulates JH1 function. (Thomas, et al., 2015 British Journal of Cancer 113, 365-371.)

These cytoplasmic tyrosine kinases are associated with membrane cytokine receptors such as common gamma-chain receptors and the glycoprotein 130 (gp130) transmembrane proteins. (Murray, et al. 2007 Immunol. 178(5):2623-2629.) About 40 cytokine receptors signal through combinations of these four JAKs and their 7 downstream substrates: the STAT family members. (Ghoreschi et al. 2009 Immunol Rev. 228(1):273-287.)

TYK2 is a key component of the JAK-STAT signaling pathway. TYK2 regulates INFα, IL12 and IL23. (Ihle, et al. 1995 Annu Rev Immunol. 13:369-398; Leonard, et al. 1998 Annu Rev Immunol. 16:293-322; Liu, et al. 1998 Curr Opin Immunol. 10:271-278.) Cytokines implicated in TYK2 activation include interferons (e.g., IFN-α, IFN-b, IFN-k, IFN-d, IFN-e, IFN-t, IFN-w, and IFN-z, and interleukins (e.g., IL-4, IL-6, IL-10, IL-11, IL-12, IL-13, L-22, IL-23, IL-27, IL-31, oncostatin M, ciliary neurotrophic factor, cardiotrophin 1, cardiotrophin-like cytokine, and LIF). The activated TYK2 goes on to phosphorylate further signaling proteins such as members of the STAT family, including STAT1, STAT2, STAT4, and STAT6. Selective inhibition of TYK2 can be utilized to treat a variety of autoimmune inflammatory diseases, such as psoriasis, systemic lupus erythematosus (SLE), inflammatory bowel disease (IBD), rheumatoid arthritis (RA), as well as cancer and diabetes.

The selectivity against other JAK family subtypes is regarded as crucial in order to increase the intended pharmacological effects and to reduce side effects. Identifying kinase inhibitors with a high degree of TYK2 selectivity has posed a significant challenge partly due to the high sequence homology of the active site among the JAK family kinases. TYK2 specificity is critical for clinical application of TYK2 kinase inhibitors, because Tyk2 knockout mice are viable with normal blood cell counts, whereas deficiency of JAK3 results in severe combined immunodeficiency in mice, and JAK1 or JAK2 knockout mice show perinatal lethality. (Ghoreschi, et al. 2009 Immunol Rev. 228:273-287; Karaghiosoff, et al. 2000 Immunity. 13:549-560; Shimoda, et al. 2000 Immunity. 13:561-571.) Genetic evidence suggests that pharmacological inhibition of TYK2 should not result in acute toxicity in human patients, but careful monitoring for viral or mycobacterial infections would be warranted in patients treated for prolonged periods. (Akahane, et al. 2017 Br J Haematol. 177(2): 271-282.)

An urgent need exists and challenges remain across broad therapeutic areas for high selective TYK2 inhibitors against other JAK family members with improved potency and minimal side effects.

### SUMMARY OF THE INVENTION

The invention provides novel, high selective and potent compounds that are orally available. These therapeutic agents are safe, effective and high selective TYK2 inhibitors and exhibit fewer and/or lesser side effects than currently available drugs. The invention also provides pharmaceutical compositions of these compounds and methods of their preparation and use.

In one aspect, the invention generally relates to a compound having the structural formula (I):

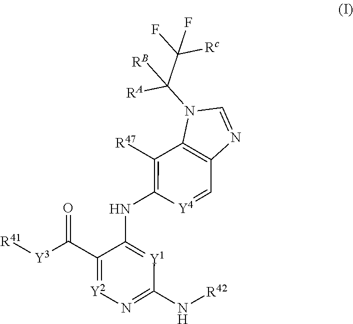

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a};

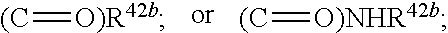

each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a};
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c};
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a}; and
R⁴⁷ is a C₁₋₃ alkoxy group optionally substituted with 1-3 D or halo.

In another aspect, the invention generally relates to a compound having the structural formula (II):

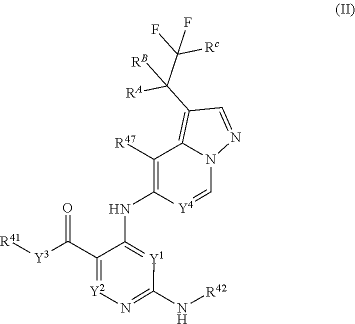

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

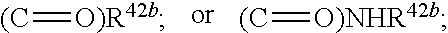

each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms
selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a}; and
R⁴⁷ is a C₁₋₃ alkoxy group optionally substituted with 1-3 D or halo.

In yet another aspect, the invention generally relates to a compound having the structural formula (III):

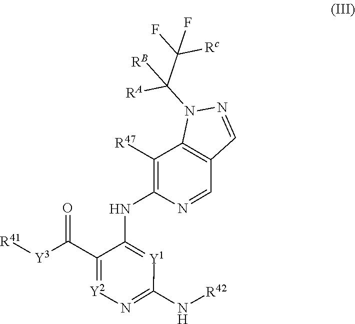

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

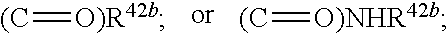

each of R^{A} and R^{B} is independently selected from H, D, halo, OR, and C₁₋₃ alkyl, provided that when both R^{A} and R^{B} is H, R^{C} is not CH₃ or F;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R⁴¹, C²⁻⁶ alkynyl substituted with 0-3 R^{42a}; and
R⁴⁷ is a C₁₋₃ alkoxy group optionally substituted with 1-3 D or halo.

In yet another aspect, the invention generally relates to a compound having the structural formula (IV):

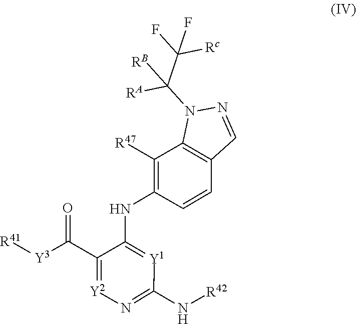

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

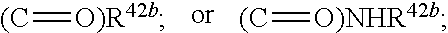

each of R^{A} and R^{B} is independently selected from H, D, halo, OR, and C₁₋₃ alkyl, provided that when both R^{A} and R^{B} is H, RC is not H or F;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42′}, C²⁻⁶ alkynyl substituted with 0-3 R^{42a}; and
R⁴⁷ is a C₁₋₃ alkoxy group optionally substituted with 1-3 D or halo.

In yet another aspect, the invention generally relates to a compound having the structural formula (V):

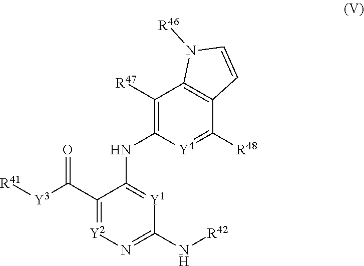

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a};

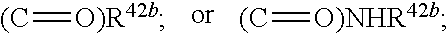

each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a};
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R⁴¹, C²⁻⁶ alkynyl substituted with 0-3 R^{42a};
R⁴⁶ is H, CD₃, a C₁-C₆ alkyl or C₃-C₆ cycloalkyl, with 0-4 of halogen, CN and OR;
R⁴⁷ is a C₁₋₃ alkoxy group optionally substituted with 1-3 D or halo; and
R⁴⁸ is H, halo or a C₁-C₃ alkyl.

In yet another aspect, the invention generally relates to a method for preparing a compound disclosed herein, as exemplified by the synthetic schemes and experimental procedure disclosed herein.

In yet another aspect, the invention generally relates to a pharmaceutical composition comprising a compound disclosed herein, effective to treat or reduce one or more diseases or disorders, in a mammal, including a human, and a pharmaceutically acceptable excipient, carrier, or diluent.

In yet another aspect, the invention generally relates to a unit dosage form comprising a pharmaceutical composition disclosed herein.

In yet another aspect, the invention generally relates to a method for treating, reducing or preventing a disease or disorder, comprising administering to a subject in need thereof a therapeutically effective amount of a compound having the structural formula of (I):

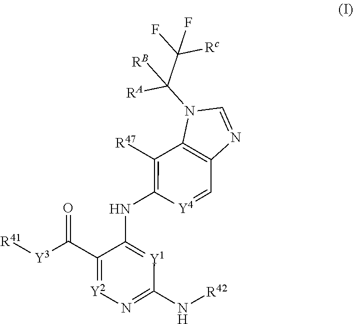

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

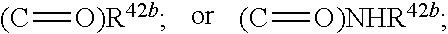

each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a};
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a};
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R⁴¹, C²⁻⁶ alkynyl substituted with 0-3 R^{42a}; and
R⁴⁷ is a C₁₋₃ alkoxy group optionally substituted with 1-3 D or halo,
   wherein the disease or disorder is selected from inflammatory diseases, immune-mediated diseases, cancer, or a related disease or disorder thereof, in a mammal, including a human.

In yet another aspect, the invention generally relates to a method for treating, reducing or preventing a disease or disorder, comprising administering to a subject in need thereof a therapeutically effective amount of a compound having the structural formula of (II):

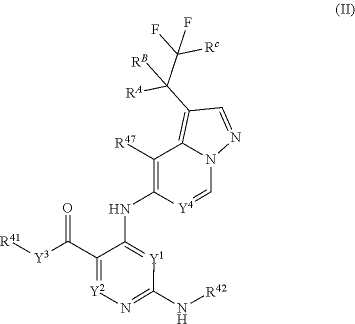

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

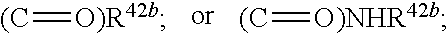

each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R⁴¹, C²⁻⁶ alkynyl substituted with 0-3 R^{42a}; and
R⁴⁷ is a C₁₋₃ alkoxy group optionally substituted with 1-3 D or halo,
   wherein the disease or disorder is selected from inflammatory diseases, immune-mediated diseases, cancer, or a related disease or disorder thereof, in a mammal, including a human.

In yet another aspect, the invention generally relates to a method for treating, reducing or preventing a disease or disorder, comprising administering to a subject in need thereof a therapeutically effective amount of a compound having the structural formula of (III):

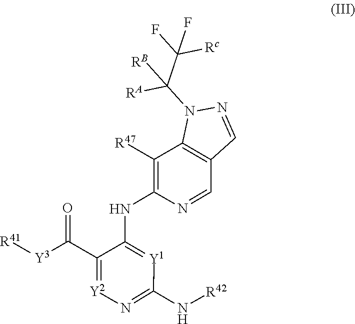

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

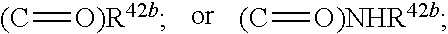

each of R^{A} and R^{B} is independently selected from H, D, halo, OR, and C₁₋₃ alkyl, provided that when both R^{A} and R^{B} is H, R^{C} is not CH₃ or F;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a}; and
R⁴⁷ is a C₁₋₃ alkoxy group optionally substituted with 1-3 D or halo,
   wherein the disease or disorder is selected from inflammatory diseases, immune-mediated diseases, cancer, or a related disease or disorder thereof, in a mammal, including a human.

In yet another aspect, the invention generally relates to a method for treating, reducing or preventing a disease or disorder, comprising administering to a subject in need thereof a therapeutically effective amount of a compound having the structural formula of (IV):

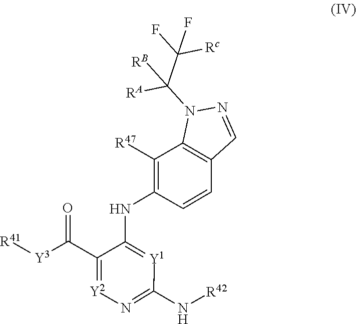

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group each substituted with 0-2 R^{42a}; or

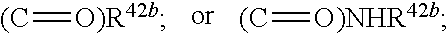

each of R^{A} and R^{B} is independently selected from H, D, halo, OR, and C₁₋₃ alkyl, provided that when both R^{A} and R^{B} is H, R^{C} is not H or F;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R⁴¹, C²⁻⁶ alkynyl substituted with 0-3 R^{42a}; and
R⁴⁷ is a C₁₋₃ alkoxy group optionally substituted with 1-3 D or halo,
   wherein the disease or disorder is selected from inflammatory diseases, immune-mediated diseases, cancer, or a related disease or disorder thereof, in a mammal, including a human.

In yet another aspect, the invention generally relates to a method for treating, reducing or preventing a disease or disorder, comprising administering to a subject in need thereof a therapeutically effective amount of a compound having the structural formula of (V):

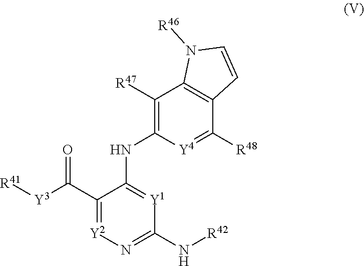

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{4a};

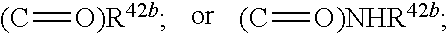

each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a};
R^{42C} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R⁴¹, C²⁻⁶ alkynyl substituted with 0-3 R^{42a};
R⁴⁶ is H, CD₃, a C₁-C₆ alkyl or C₃-C₆ cycloalkyl, with 0-4 of halogen, CN and OR;
R⁴⁷ is a C₁₋₃ alkoxy group optionally substituted with 1-3 D or halo; and
R⁴¹ is H, halo or a C₁-C₃ alkyl,
   wherein the disease or disorder is selected from inflammatory diseases, immune-mediated diseases, cancer, or a related disease or disorder thereof, in a mammal, including a human.

In yet another aspect, the invention generally relates to use of a compound disclosed herein, and a pharmaceutically acceptable excipient, carrier, or diluent, in preparation of a medicament for treating a disease or disorder.

### Definitions

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

The following terms, unless indicated otherwise according to the context wherein the terms are found, are intended to have the following meanings.

Ranges provided herein are understood to be shorthand for all of the values within the range. For example, a range of 1 to 16 is understood to include any number, combination of numbers, or sub-range from the group consisting 1, 2, 3, 4, 5, 6, 7, 8, 9, 10, 11, 12, 13, 14, 15 or 16.

As used herein, “at least” a specific value is understood to be that value and all values greater than that value.

As used herein, “more than one” is understood as 2, 3, 4, 5, 6, 7, 8, 9, 10, 11, 12, 13, 14, 15, 16, 17, 18, 19, 20, 25, 30, 40, 50, 100, etc., or any value therebetween.

In this specification and the appended claims, the singular forms “a,” “an,” and “the” include plural reference, unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Unless specifically stated or obvious from context, as used herein, the term “or” is understood to be inclusive.

Any compositions or methods disclosed herein can be combined with one or more of any of the other compositions and methods provided herein.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Certain compounds of the present invention may exist in particular geometric or stereoisomeric forms. The present invention contemplates all such compounds, including cis- and trans-isomers, atropisomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess of either the R- or S-configuration. For optically active compounds, it is often preferred to use one enantiomer to the substantial exclusion of the other enantiomer.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

A mixture of isomers can be separated on the basis of the physicochemical differences of the constituents, into the pure or substantially pure geometric or optical isomers, diastereomers, racemates, for example, by chromatography and/or fractional crystallization.

Definitions of specific functional groups and chemical terms are described in more detail below. When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

Where substituent groups are specified by their conventional chemical formulae, written from left to right, they equally encompass the chemically identical substituents that would result from writing the structure from right to left, e.g., —C(═O)—O— is equivalent to —O—C(═O)—.

Structures of compounds of the invention are limited by principles of chemical bonding known to those skilled in the art. Accordingly, where a group may be substituted by one or more of a number of substituents, such substitutions are selected so as to comply with principles of chemical bonding and to give compounds that are not inherently unstable and/or would be known to one of ordinary skill in the art as likely to be unstable under ambient conditions (e.g., aqueous, neutral, and several known physiological conditions).

Solvates and polymorphs of the compounds of the invention are also contemplated herein. Solvates of the compounds of the present invention include, for example, hydrates.

As used herein, the term “alkyl” refers to a straight or branched hydrocarbon chain radical consisting solely of carbon and hydrogen atoms, containing no unsaturation, having from one to ten carbon atoms (e.g., C₁₋₁₀ alkyl). Whenever it appears herein, a numerical range such as “1 to 10” refers to each integer in the given range; e.g., “1 to 10 carbon atoms” means that the alkyl group can consist of 1 carbon atom, 2 carbon atoms, 3 carbon atoms, etc., up to and including 10 carbon atoms, although the present definition also covers the occurrence of the term “alkyl” where no numerical range is designated. In some embodiments, “alkyl” can be a C₁₋₆ alkyl group. In some embodiments, alkyl groups have 1 to 10, 1 to 8, 1 to 6, or 1 to 3 carbon atoms. Representative saturated straight chain alkyls include, but are not limited to, -methyl, -ethyl, -n-propyl, -n-butyl, -n-pentyl, and -n-hexyl; while saturated branched alkyls include, but are not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The alkyl is attached to the parent molecule by a single bond. Unless stated otherwise in the specification, an alkyl group is optionally substituted by one or more of substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR*, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)N(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. In a non-limiting embodiment, a substituted alkyl can be selected from fluoromethyl, difluoromethyl, trifluoromethyl, 2-fluoroethyl, 3-fluoropropyl, hydroxymethyl, 2-hydroxyethyl, 3-hydroxypropyl, benzyl, and phenethyl.

As used herein, the term “alkoxy” refers to the group —O-alkyl, including from 1 to 10 carbon atoms (C₁₋₁₀) of a straight, branched, saturated cyclic configuration and combinations thereof, attached to the parent molecular structure through an oxygen. Unless stated otherwise in the specification, the term is intended to include both substituted and unsubstituted alkoxy groups. Examples include methoxy, ethoxy, propoxy, isopropoxy, butoxy, t-butoxy, pentoxy, cyclopropyloxy, cyclohexyloxy and the like. “Lower alkoxy” refers to alkoxy groups containing one to six carbons. In some embodiments, C₁₋₃ alkoxy is an alkoxy group that encompasses both straight and branched chain alkyls of from 1 to 3 carbon atoms. Unless stated otherwise in the specification, an alkoxy group can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR′, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)N(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein.

As used herein, the terms “aromatic” or “aryl” refer to a radical with 6 to 14 ring atoms (e.g., C₆₋₁₄ aromatic or C₆₋₁₄ aryl) that has at least one ring having a conjugated pi electron system which is carbocyclic (e.g., phenyl, fluorenyl, and naphthyl). Unless stated otherwise in the specification, the term is intended to include both substituted and unsubstituted aryl groups. In some embodiments, the aryl is a C₆₋₁₀ aryl group. For example, bivalent radicals formed from substituted benzene derivatives and having the free valences at ring atoms are named as substituted phenylene radicals. In other embodiments, bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 14 aryl” refers to each integer in the given range; e.g., “6 to 14 ring atoms” means that the aryl group can consist of 6 ring atoms, 7 ring atoms, etc., up to and including 14 ring atoms. The term includes monocyclic or fused-ring polycyclic (i.e., rings which share adjacent pairs of ring atoms) groups. Polycyclic aryl groups include bicycles, tricycles, tetracycles, and the like. In a multi-ring group, only one ring is required to be aromatic, so groups such as indanyl are encompassed by the aryl definition. Non-limiting examples of aryl groups include phenyl, phenalenyl, naphthalenyl, tetrahydronaphthyl, phenanthrenyl, anthracenyl, fluorenyl, indolyl, indanyl, and the like. Unless stated otherwise in the specification, an aryl moiety can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR′, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)N(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein.

As used herein, the terms “cycloalkyl” and “carbocyclyl” each refers to a monocyclic or polycyclic radical that contains only carbon and hydrogen, and can be saturated or partially unsaturated. Partially unsaturated cycloalkyl groups can be termed “cycloalkenyl” if the carbocycle contains at least one double bond, or “cycloalkynyl” if the carbocycle contains at least one triple bond. Cycloalkyl groups include groups having from 3 to 13 ring atoms (i.e., C₃₋₁₃ cycloalkyl). Unless stated otherwise in the specification, the term is intended to include both substituted and unsubstituted cycloalkyl groups. Whenever it appears herein, a numerical range such as “3 to 10” refers to each integer in the given range; e.g., “3 to 13 carbon atoms” means that the cycloalkyl group can consist of 3 carbon atoms, 4 carbon atoms, 5 carbon atoms, etc., up to and including 13 carbon atoms. The term “cycloalkyl” also includes bridged and spiro-fused cyclic structures containing no heteroatoms. The term also includes monocyclic or fused-ring polycyclic (i.e., rings which share adjacent pairs of ring atoms) groups. Polycyclic aryl groups include bicycles, tricycles, tetracycles, and the like. In some embodiments, “cycloalkyl” can be a C₃₋₈ cycloalkyl radical. In some embodiments, “cycloalkyl” can be a C₃₋₅ cycloalkyl radical. Illustrative examples of cycloalkyl groups include, but are not limited to the following moieties: C₃₋₆ carbocyclyl groups include, without limitation, cyclopropyl (C₃), cyclobutyl (C₄), cyclopentyl (C₅), cyclopentenyl (C₅), cyclohexyl (C₆), cyclohexenyl (C₆), cyclohexadienyl (C₆) and the like. Examples of C₃₋₇ carbocyclyl groups include norbornyl (C₇). Examples of C₃₋₈ carbocyclyl groups include the aforementioned C₃₋₇ carbocyclyl groups as well as cycloheptyl (C₇), cycloheptadienyl (C₇), cycloheptatrienyl (C₇), cyclooctyl (C₅), bicyclo[2.2.1]heptanyl, bicyclo[2.2.2]octanyl, and the like. Examples of C₃₋₁₃ carbocyclyl groups include the aforementioned C₃₋₈ carbocyclyl groups as well as octahydro-1H indenyl, decahydronaphthalenyl, spiro[4.5]decanyl and the like. Unless stated otherwise in the specification, a cycloalkyl group can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)N(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. The terms “cycloalkenyl” and “cycloalkynyl” mirror the above description of “cycloalkyl” wherein the prefix “alk” is replaced with “alken” or “alkyn” respectively, and the parent “alkenyl” or “alkynyl” terms are as described herein. For example, a cycloalkenyl group can have 3 to 13 ring atoms, such as 5 to 8 ring atoms. In some embodiments, a cycloalkynyl group can have 5 to 13 ring atoms.

As used herein, the term “halogen” refers to fluorine (F), chlorine (Cl), bromine (Br), or iodine (I). As used herein, the term “halide” or “halo”, means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine, such as, but not limited to, trifluoromethyl, difluoromethyl, 2,2,2-trifluoroethyl, 1-fluoromethyl-2-fluoroethyl, and the like. Each of the alkyl, alkenyl, alkynyl and alkoxy groups are as defined herein and can be optionally further substituted as defined herein.

As used herein, the term “heteroatom” refers to oxygen (O), nitrogen (N), sulfur (S), and phosphorus (P).

As used herein, the term “heteroalkyl” refers to an alkyl radical, which have one or more skeletal chain atoms selected from an atom other than carbon, e.g., oxygen, nitrogen, sulfur, phosphorus or combinations thereof. Unless stated otherwise in the specification, the term is intended to include both substituted and unsubstituted heteroalkyl groups. A numerical range can be given, e.g., C₁₋₄ heteroalkyl, which refers to the chain length in total, which in this example is 4 atoms long. For example, a —CH₂OCH₂CH₃ radical is referred to as a “C₄” heteroalkyl, which includes the heteroatom center in the atom chain length description. Connection to the parent molecular structure can be through either a heteroatom or a carbon in the heteroalkyl chain. For example, an N-containing heteroalkyl moiety refers to a group in which at least one of the skeletal atoms is a nitrogen atom. One or more heteroatom(s) in the heteroalkyl radical can be optionally oxidized. One or more nitrogen atoms, if present, can also be optionally quaternized. For example, heteroalkyl also includes skeletal chains substituted with one or more nitrogen oxide (—O—) substituents. Exemplary heteroalkyl groups include, without limitation, ethers such as methoxyethanyl (—CH₂CH₂OCH₃), ethoxymethanyl (—CH₂OCH₂CH₃), (methoxymethoxy)ethanyl (—CH₂CH₂OCH₂OCH₃), (methoxymethoxy) methanyl (—CH₂OCH₂OCH₃) and (methoxyethoxy)methanyl (—CH₂OCH₂CH₂OCH₃) and the like; amines such as (—CH₂CH₂NHCH₃, —CH₂CH₂N(CH₃)₂, —CH₂NHCH₂CH₃, —CH₂N(CH₂CH₃)(CH₃)) and the like.

As used herein, the term “heterocycloalkyl” refers to a cycloalkyl radical, which have one or more skeletal chain atoms selected from an atom other than carbon, e.g., oxygen, nitrogen, sulfur, phosphorus or combinations thereof. Unless stated otherwise in the specification, the term is intended to include both substituted and unsubstituted heterocycloalkyl groups. Illustrative examples of heterocycloalkyl include 2-hydroxy-aziridin-1-yl, 3-oxo-1-oxacyclobutan-2-yl, 2,2-dimethyl-tetrahydrofuran-3-yl, 3-carboxy-morpholin-4-yl, 1-cyclopropyl-4-methyl-piperazin-2-yl. 2-pyrrolinyl, 3-pyrrolinyl, dihydro-2H-pyranyl, 1,2,3,4-tetrahydropyridine, 3,4-dihydro-2H-[1,4]oxazine, etc.

As used herein, the term “heteroaryl” or, alternatively, “heteroaromatic” refers to a radical of a 5-18 membered monocyclic or polycyclic (e.g., bicyclic, tricyclic, tetracyclic and the like) aromatic ring system (e.g., having 6, 10 or 14 π electrons shared in a cyclic array) having ring carbon atoms and 1-6 ring heteroatoms provided in the aromatic ring system, wherein each heteroatom is independently selected from nitrogen, oxygen, phosphorous and sulfur (“5-18 membered heteroaryl”). Unless stated otherwise in the specification, the term is intended to include both substituted and unsubstituted heteroaryl groups. Heteroaryl polycyclic ring systems can include one or more heteroatoms in one or both rings. Whenever it appears herein, a numerical range such as “5 to 18” refers to each integer in the given range; e.g., “5 to 18 ring atoms” means that the heteroaryl group can consist of 5 ring atoms, 6 ring atoms, etc., up to and including 18 ring atoms. In some instances, a heteroaryl can have 5 to 14 ring atoms. In some embodiments, the heteroaryl has, for example, bivalent radicals derived from univalent heteroaryl radicals whose names end in “-yl” by removal of one hydrogen atom from the atom with the free valence are named by adding “-ene” to the name of the corresponding univalent radical, e.g., a pyridyl group with two points of attachment is a pyridylene.

For example, an N-containing “heteroaromatic” or “heteroaryl” moiety refers to an aromatic group in which at least one of the skeletal atoms of the ring is a nitrogen atom. One or more heteroatom(s) in the heteroaryl radical can be optionally oxidized. One or more nitrogen atoms, if present, can also be optionally quaternized. Heteroaryl also includes ring systems substituted with one or more nitrogen oxide (—O—) substituents, such as pyridinyl N-oxides. The heteroaryl is attached to the parent molecular structure through any atom of the ring(s).

“Heteroaryl” also includes ring systems wherein the heteroaryl ring, as defined above, is fused with one or more aryl groups wherein the point of attachment to the parent molecular structure is either on the aryl or on the heteroaryl ring, or wherein the heteroaryl ring, as defined above, is fused with one or more cycloalkyl or heterocycyl groups wherein the point of attachment to the parent molecular structure is on the heteroaryl ring. For polycyclic heteroaryl groups wherein one ring does not contain a heteroatom (e.g., indolyl, quinolinyl, carbazolyl and the like), the point of attachment to the parent molecular structure can be on either ring, i.e., either the ring bearing a heteroatom (e.g., 2-indolyl) or the ring that does not contain a heteroatom (e.g., 5-indolyl). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, phosphorous, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, phosphorous, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, phosphorous, and sulfur (“5-6 membered heteroaryl”). In some embodiments, the 5-6 membered heteroaryl has 1-3 ring heteroatoms selected from nitrogen, oxygen, phosphorous, and sulfur. In some embodiments, the 5-6 membered heteroaryl has 1-2 ring heteroatoms selected from nitrogen, oxygen, phosphorous, and sulfur. In some embodiments, the 5-6 membered heteroaryl has 1 ring heteroatom selected from nitrogen, oxygen, phosphorous, and sulfur.

Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzopyranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimdinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e., thienyl). Unless stated otherwise in the specification, a heteroaryl moiety can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)N(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein.

As used herein, the term “administering” refers to oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Suitable routes of administration for a particular patient will depend on the nature and severity of the disease or condition being treated or the nature of the therapy being used and on the nature of the active compound.

Administration may be by any suitable route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

By “co-administer” it is meant that a composition described herein is administered at the same time, just prior to, or just after the administration of one or more additional therapies.

The compound of the invention can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The compositions of the present invention can be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, gels, for example, water or water/propylene glycol solutions.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, 1995 J. Biomater Sci. Polym. Ed. 7:623-645; as biodegradable and injectable gel formulations (see, e.g., Gao 1995 Pharm. Res. 12:857-863); or, as microspheres for oral administration (see, e.g., Eyles 1997 J. Pharm. Pharmacol. 49:669-674).

As used herein, the terms “disease,” “condition,” and “disorder” are used interchangeably herein and refer to a state of being or health status of a patient or subject capable of being treated with a compound, pharmaceutical composition, or method provided herein.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the terms “inhibition,” “inhibit” and “inhibiting” and the like in reference to a biological target (e.g., TYK2) inhibitor interaction refers to negatively affecting (e.g., decreasing) the activity or function of the protein relative to the activity or function of the protein in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

As used herein, the terms “isolated” or “purified” refer to a material that is substantially or essentially free from components that normally accompany it in its native state. Purity and homogeneity are typically determined using analytical chemistry techniques such as polyacrylamide gel electrophoresis or high-performance liquid chromatography.

As used herein, the term “modulate” refers to the production, either directly or indirectly, of an increase or a decrease, a stimulation, inhibition, interference, or blockage in a measured activity when compared to a suitable control. A “modulator” of a polypeptide or polynucleotide refers to a substance that affects, for example, increases, decreases, stimulates, inhibits, interferes with, or blocks a measured activity of the polypeptide or polynucleotide, when compared to a suitable control. For example, a “modulator” may bind to and/or activate or inhibit the target with measurable affinity, or directly or indirectly affect the normal regulation of a receptor activity.

As used herein, a “pharmaceutically acceptable form” of a disclosed compound includes, but is not limited to, pharmaceutically acceptable salts, esters, hydrates, solvates, isomers, prodrugs, and isotopically labeled derivatives thereof. In one embodiment, a “pharmaceutically acceptable form” includes, but is not limited to, pharmaceutically acceptable salts, esters, prodrugs and isotopically labeled derivatives thereof. In some embodiments, a “pharmaceutically acceptable form” includes, but is not limited to, pharmaceutically acceptable isomers and stereoisomers, prodrugs and isotopically labeled derivatives thereof.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchlorate acid or with organic acids such as acetic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, p-toluenesulfonate, undecanoate, valerate salts, and the like. In some embodiments, organic acids from which salts can be derived include, for example, acetic acid, propionic acid, glycolic acid, pyruvic acid, lactic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate.” Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

Prodrug forms often offer advantages of solubility, tissue compatibility, or delayed release in the mammalian organism. (See, Bundgard, Design of Prodrugs, pp. 7-9,21-24, Elsevier, Amsterdam 1985 and Silverman, The Organic Chemistry of Drug Design and Drug Action, pp. 352-401, Academic Press, San Diego, Calif., 1992.) Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Other prodrug derivatives may be combined with other features disclosed herein to enhance bioavailability. As such, those of skill in the art will appreciate that certain of the presently disclosed compounds having free amino, amido, hydroxy or carboxylic groups can be converted into prodrugs. Prodrugs include compounds having a carbonate, carbamate, amide or alkyl ester moiety covalently bonded to any of the above substituents disclosed herein.

Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “subject” refers to any animal (e.g., a mammal), including, but not limited to humans, non-human primates, rodents, and the like, which is to be the recipient of a particular treatment. A subject to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), rodents (e.g., rats and/or mice), etc. In certain embodiments, the non-human animal is a mammal. The non-human animal may be a male or female at any stage of development. A non-human animal may be a transgenic animal. Typically, the terms “subject” and “patient” are used interchangeably herein in reference to a human subject.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. The treatment can be any reduction and can be, but is not limited to, the complete ablation of the disease or the symptoms of the disease. Treating or treatment thus refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, for example, the results of a physical examination, neuropsychiatric exams, and/or a psychiatric evaluation. As compared with an equivalent untreated control, such reduction or degree of amelioration may be at least 5%, 10%, 20%, 40%, 50%, 60%, 80%, 90%, 95%, or 100% as measured by any standard technique.

Treatment methods include administering to a subject a therapeutically effective amount of a compound described herein. The administering step may be a single administration or may include a series of administrations. The length of the treatment period depends on a variety of factors, such as the severity of the condition, the patient's age, the concentration of the compound, the activity of the compositions used in the treatment, or a combination thereof. It will also be appreciated that the effective dosage of an agent used for the treatment may increase or decrease over the course of a particular treatment regime. Changes in dosage may result and become apparent by standard diagnostic assays known in the art. In some instances, chronic administration may be required. For example, the compositions are administered to the subject in an amount and for a duration sufficient to treat the patient.

### DETAILED DESCRIPTION OF THE INVENTION

The invention is based on an unexpected discovery of novel, selective and potent compounds that are TYK2 inhibitors. The invention also provides pharmaceutical compositions of these compounds and methods of their preparation and use. The compounds are orally available and exhibit fewer and/or lesser side effects than currently available drugs.

The new class of TYK2 inhibitors disclosed herein exhibit exceptional potency profiles and are useful in treating one or more TYK2-mediated diseases and conditions, such as allergic, autoimmune, inflammatory, metabolic, neurological and proliferative diseases and conditions. Without wishing to be bound by the theory, compounds of the invention are modulators of interleukins (e.g., IL-12, IL-23) and interferons (e.g., IFN-a) by inhibiting TYK2-mediated signal transduction.

These compounds are designed to show good potency against TYK2 with good oral absorption and good in vivo stability. The invention also provides pharmaceutical compositions of these compounds and methods of preparation and use thereof. The TYK2 inhibitors disclosed herein exhibit favorable pharmacokinetic profiles and drug properties that are suitable for the target indications.

In one aspect, the invention generally relates to a compound having the structural formula (I):

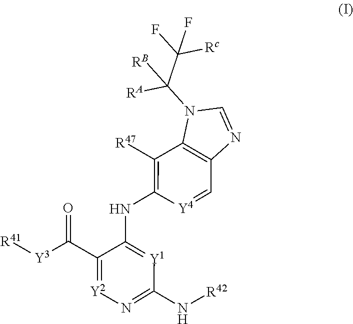

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a};

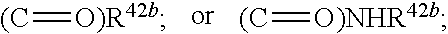

each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a};
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a};
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R⁴¹, C²⁻⁶ alkynyl substituted with 0-3 R^{42a}; and
R⁴⁷ is a C₁₋₃ alkoxy group optionally substituted with 1-3 D or halo.

In certain embodiments of (I), Y⁴ is CH and the compound has the structure:

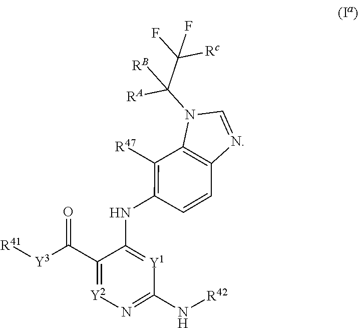

In certain embodiments of (I), Y¹ is CH and Y² is CH, having the structure:

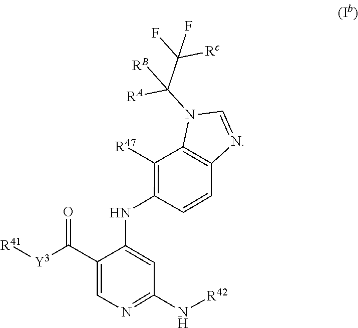

In certain embodiments of (I), Y¹ is N and Y² is CH, having the structure:

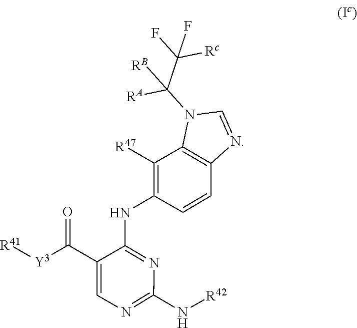

In certain embodiments of (I), Y¹ is CH and Y² is N, having the structure:

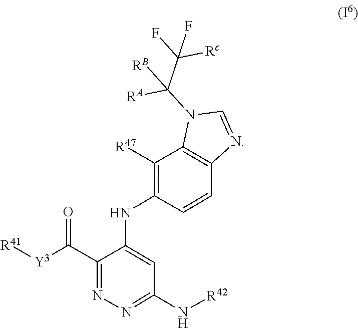

In certain embodiments of (I), Y¹ is N and Y² is N, having the structure:

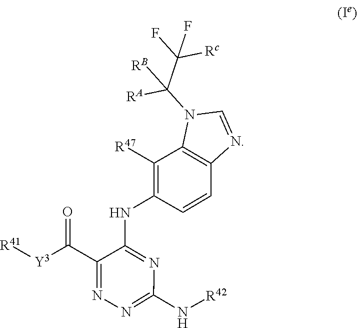

In certain embodiments of (I), Y⁴ is N, having the structure:

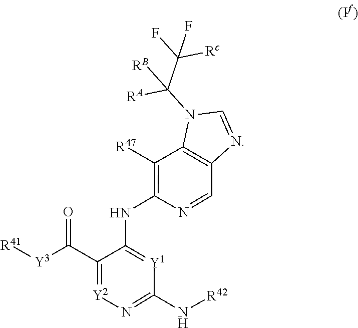

In certain embodiments of (I), Y¹ is CH and Y² is CH, having the structure:

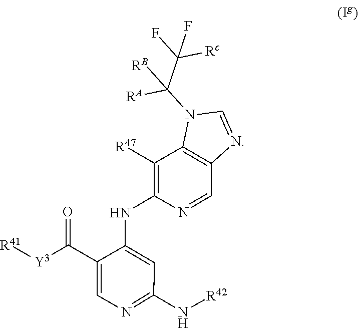

In certain embodiments of (I^{f}), Y¹ is N and Y² is CH, having the structure:

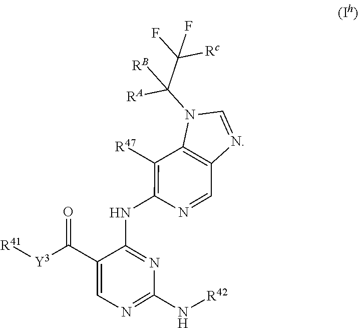

In certain embodiments of (I^{f}), Y¹ is CH and Y² is N, having the structure:

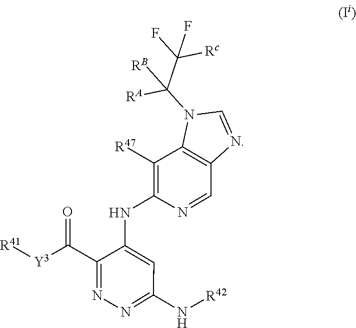

In certain embodiments of (I^{f}), Y¹ is N and Y² is N, having the structure:

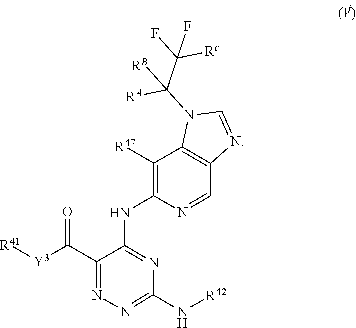

In certain embodiments of (I)-(I^{j}), R^{A} is H and R^{B} is H.

In certain embodiments of (I)-(I^{j}), R^{C} is CH₃.

In certain embodiments of (I)-(I^{j}), R^{C} is not CH₃.

In another aspect, the invention generally relates to a compound having the structural formula (II):

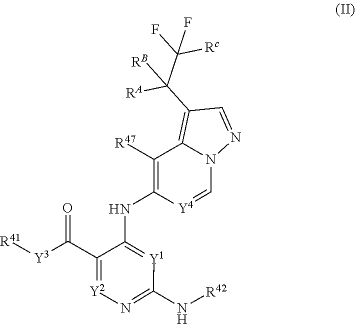

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

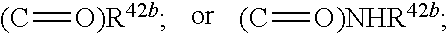

each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42′}, C²⁻⁶ alkynyl substituted with 0-3 R^{42a}; and
R⁴⁷ is a C₁₋₃ alkoxy group optionally substituted with 1-3 D or halo.

In certain embodiments of (II), Y⁴ is CH, having the structure:

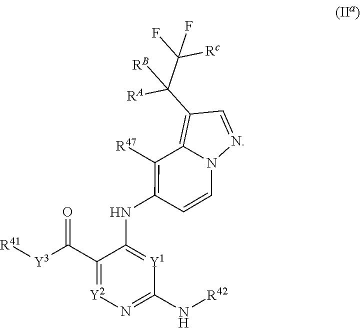

In certain embodiments of (II^{a}), Y¹ is CH and Y² is CH, having the structure:

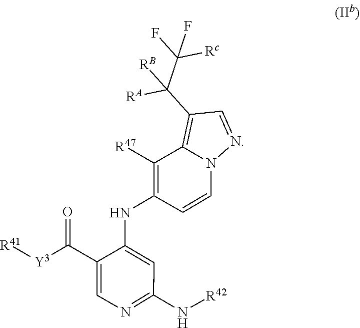

In certain embodiments of (II^{a}), Y¹ is CH and Y² is N, having the structure:

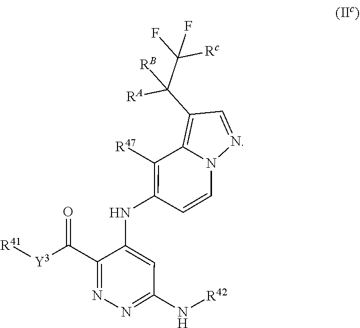

In certain embodiments of (II^{a}), Y¹ is N and Y² is CH, having the structure:

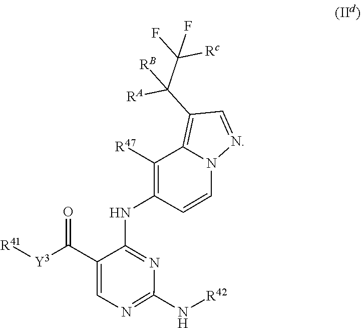

In certain embodiments of (II^{a}), Y¹ is N and Y² is N, having the structure:

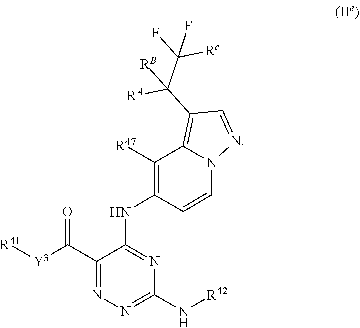

In certain embodiments of (II), Y⁴ is N, having the structure:

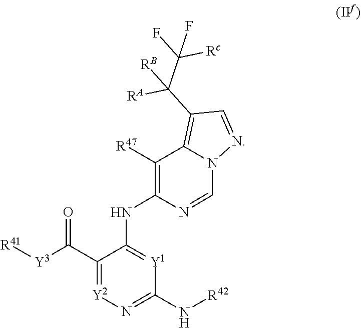

In certain embodiments of (II^{f}), Y¹ is CH and Y² is CH, having the structure:

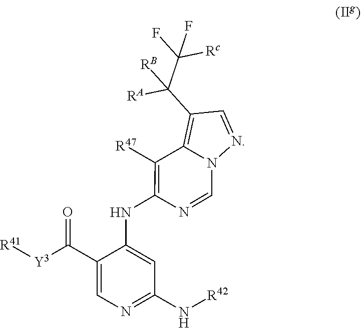

In certain embodiments of (II^{f}), Y¹ is N and Y² is CH, having the structure:

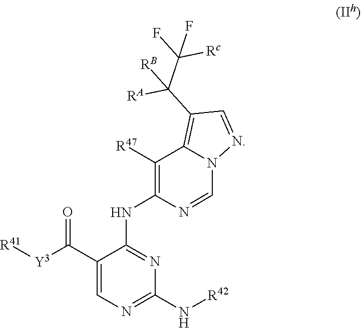

In certain embodiments of (II^{f}), Y¹ is CH and Y² is N, having the structure:

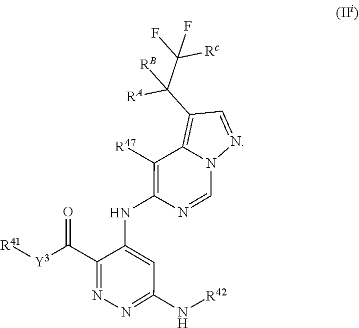

In certain embodiments of (II^{f}), Y¹ is N and Y² is N, having the structure:

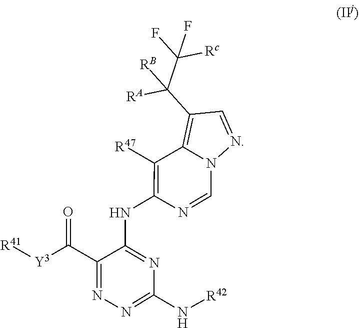

In certain embodiments of (II)-(II^{j}), R^{A} is H and R^{B} is H.

In certain embodiments of (II)-(II^{j}), R^{C} is CH₃.

In certain embodiments of (II)-(II^{j}), R^{C} is not CH₃.

In yet another aspect, the invention generally relates to a compound having the structural formula (III):

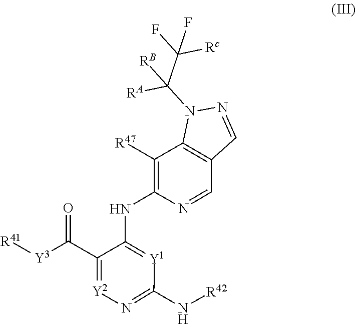

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

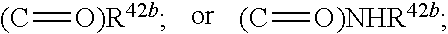

each of R^{A} and R^{B} is independently selected from H, D, halo, OR, and C₁₋₃ alkyl, provided that when both R^{A} and R^{B} is H, R^{C} is not CH₃ or F;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R⁴¹, C²⁻⁶ alkynyl substituted with 0-3 R^{42a}; and
R⁴⁷ is a C₁₋₃ alkoxy group optionally substituted with 1-3 D or halo.

In certain embodiments of (III), Y¹ is CH and Y² is CH, having the structure:

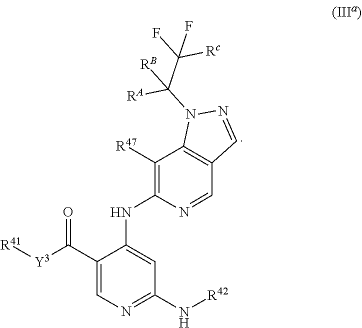

In certain embodiments of (III), Y¹ is CH and Y² is N, having the structure:

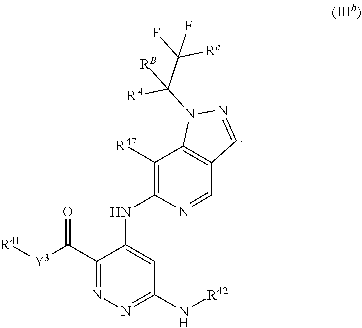

In certain embodiments of (III), Y¹ is N and Y² is CH, having the structure:

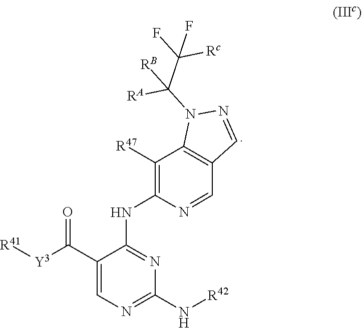

In certain embodiments of (III), Y¹ is N and Y² is N, having the structure:

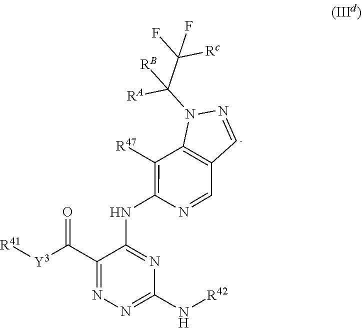

In certain embodiments of (III)-(III^{d}), R^{A} is H and R^{B} is H.

In certain embodiments of (III)-(III^{d}), one of R^{A} and R^{B} is not H.

In certain embodiments of (III)-(III^{d}), R^{C} is not CH₃.

In yet another aspect, the invention generally relates to a compound having the structural formula (IV):

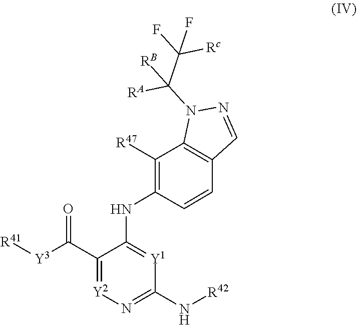

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

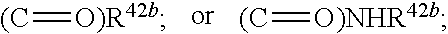

each of R^{A} and R^{B} is independently selected from H, D, halo, OR, and C₁₋₃ alkyl, provided that when both R^{A} and R^{B} is H, R^{C} is not H or F;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R⁴¹, C²⁻⁶ alkynyl substituted with 0-3 R^{42a}; and
R⁴⁷ is a C₁₋₃ alkoxy group optionally substituted with 1-3 D or halo.

In certain embodiments of (IV), Y¹ is CH and Y² is CH, having the structure:

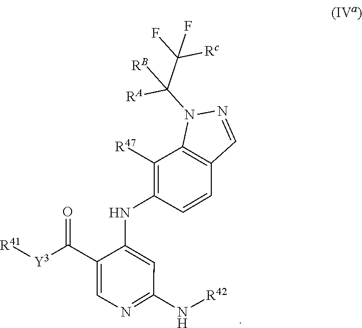

In certain embodiments of (IV), Y¹ is N and Y² is CH, having the structure:

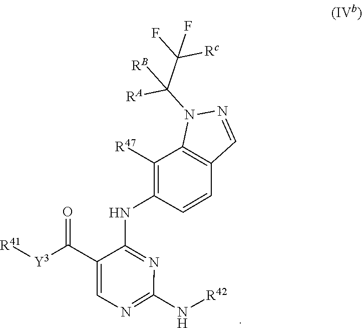

In certain embodiments of (IV), Y¹ is CH and Y² is N, having the structure:

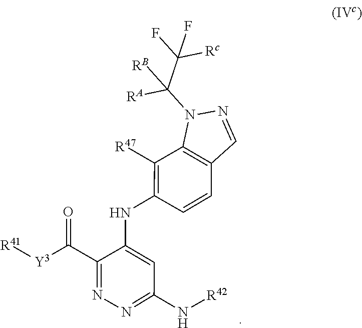

In certain embodiments of (IV), Y¹ is N and Y² is N, having the structure:

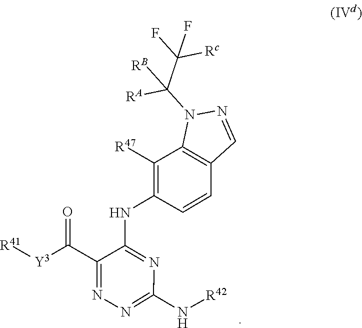

In certain embodiments of (IV)—(IV^{d}), R^{A} is H and R^{B} is H.

In certain embodiments of (IV)—(IV^{d}), R^{C} is CH₃.

In certain embodiments of (IV)—(IV^{d}), R^{C} is not CH₃.

In yet another aspect, the invention generally relates to a compound having the structural formula (V):

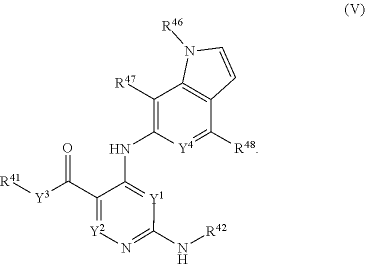

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a};

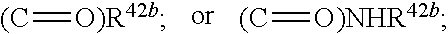

each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c};
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{4a}, C²⁻⁶ alkynyl substituted with 0-3 R^{42a};
R⁴⁶ is H, CD₃, a C₁-C₆ alkyl or C₃-C₆ cycloalkyl, with 0-4 of halogen, CN and OR;
R⁴⁷ is a C₁₋₃ alkoxy group optionally substituted with 1-3 D or halo; and
R⁴⁸ is H, halo or a C₁-C₃ alkyl.

In certain embodiments of (V), YI is CH and Y² is CH, having the structure:

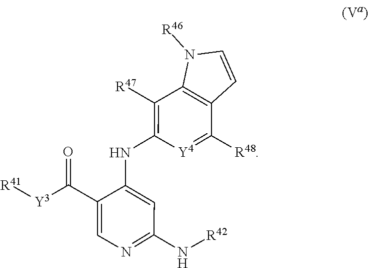

In certain embodiments of (V), Y¹ is N and Y² is CH, having the structure:

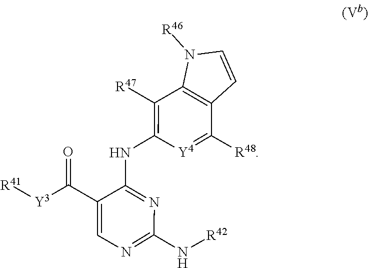

In certain embodiments of (V), Y¹ is CH and Y² is N, having the structure:

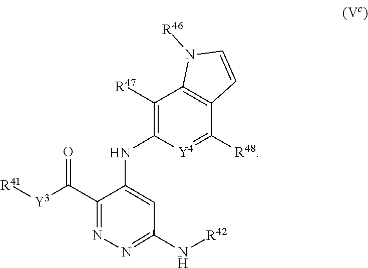

In certain embodiments of (V), Y¹ is N and Y² is N, having the structure:

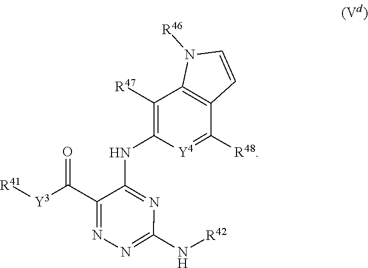

In certain embodiments of (V), the compound has the structure:

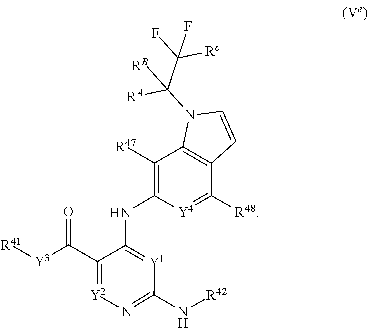

wherein
each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a};

In certain embodiments of (V)—(V^{e}), Y⁴ is CH.

In certain embodiments of (V)—(V^{e}), Y⁴ is N.

In certain embodiments of (V)—(V^{e}), R⁴⁸ is H.

In certain embodiments of (V)—(V^{e}), R⁴⁸ is halo.

In certain embodiments of (V^{e}), R^{A} is H and R^{B} is H.

In certain embodiments of (V^{e}), R^{C} is CH₃.

In certain embodiments of (V^{e}), R^{C} is not CH₃.

In certain embodiments of (I)—(V^{e}), R⁴⁷ is C₁-C₃ alkoxy.

In certain embodiments of (I)—(V^{e}), R⁴⁷ is OCH₃.

In certain embodiments of (I)—(V^{e}), R⁴⁷ is OCD₃.

In certain embodiments of (I)—(V^{e}), Y³ is NR.

In certain embodiments of (I)—(V^{e}), Y³ is NH.

In certain embodiments of (I)—(V^{e}), Y³ is O.

In certain embodiments of (I)—(V^{e}), Y³ is O—NH.

In certain embodiments of (I)—(V^{e}), R⁴¹ is CH₃.

In certain embodiments of (I)—(V^{e}), R⁴¹ is CD₃.

In certain embodiments of (I)—(V^{e}), R⁴² is R^{42′}.

In certain embodiments of (I)—(V^{e}), R⁴² is (C═O)R^{42b}, wherein R^{42b} is selected from C₁-C₆ alkyl, cyclopropyl or cyclobutyl, substituted with 0-2 R^{42c}.

In certain embodiments, R⁴² is (C═O)R^{42b}, wherein R^{42b} is cyclopropyl, optionally substituted with one or more of F, Cl, CH₃, CF₃ and CN.

In certain embodiments, R⁴² is (C═O)R^{42b}, wherein R^{42b} is bis-spiro-cyclopropyl, optionally substituted with one or more of F, Cl, CH₃, CF₃ and CN.

In certain embodiments, R⁴² is (C═O)R^{42b}, wherein R^{42b} is cyclobutyl, optionally substituted with one or more of F, Cl, CH₃, CF₃ and CN.

In certain embodiments, R⁴² is (C═O)R^{42b}, wherein R^{42b} is C₁-C₆ alkyl, optionally substituted with one or more of F, Cl, CH₃, CF₃, CN, NRR′ and OR.

In certain embodiments of (I)—(V^{e}), R⁴² is (C═O)NHR^{42b}, wherein R^{42b} is selected from C₁-C₆ alkyl, cyclopropyl or cyclobutyl, substituted with 0-2 R^{42c}.

In certain embodiments of (I)—(V^{e}), R⁴² is phenyl substituted with 0-2 R^{42c}.

In certain embodiments of (I)—(V^{e}), R⁴² is pyridinyl substituted with 0-2 R^{42c}.

In certain embodiments of (I)—(V^{e}), R⁴² is pyrazolyl substituted with 0-2 R^{42c}.

In certain embodiments of (I)—(V^{e}), R⁴² is pyrimidyl substituted with 0-2 R^{42c}.

In certain embodiments, R^{42c} is CH₃ or CD₃.

In certain embodiments, R^{42c} is F.

Non-limiting examples of compounds of the invention include those listed in Table 1A in the Examples section herein, or a pharmaceutically acceptable form or an isotope derivative thereof.

In certain embodiments, a compound of the invention is selected from those listed in Table 1B. or a pharmaceutically acceptable form or an isotope derivative thereof.

TABLE 1B

| Example | Structure | Name |
|---|---|---|
| 4 | 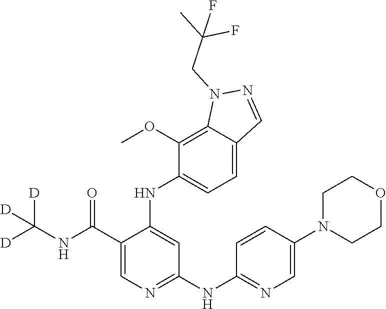 [SMILES: [2H]C([2H])([2H])NC(=O)c1cnc(Nc2ccc(N3CCOCC3)cn2)cc1Nc1ccc2cnn(CC(C)(F)F)c2c1OC] | 4-((1-(2,2-Difluoropropyl)-7- methoxy-1H-indazol-6-yl)amino)- N-(methyl-d3)-6-((5- morpholinopyridin-2- yl)amino)nicotinamide |
| 5B | 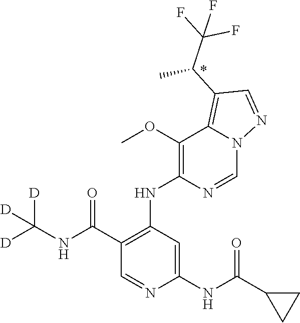 [SMILES: [2H]C([2H])([2H])NC(=O)c1cnc(NC(=O)C2CC2)cc1Nc1ncn2ncc([C@H](C)C(F)(F)F)c2c1OC] | (S*)6-(Cyclopropanecarboxamido)- 4-((4-methoxy-3-(1,1,1- trifluoropropan-2-yl)pyrazolo[1,5- c]pyrimidin-5-yl)amino)-N- (methyl-d3)nicotinamide |
| 8 | 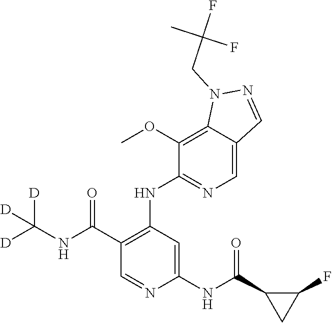 [SMILES: [2H]C([2H])([2H])NC(=O)c1cnc(NC(=O)[C@@H]2C[C@@H]2F)cc1Nc1ncc2cnn(CC(C)(F)F)c2c1OC] | 4-((1-(2,2-Difluoropropyl)-7- methoxy-1H-pyrazolo[4,3- c]pyridin-6-yl)amino)-6-((1S,2S)- 2-fluorocyclopropane-1- carboxamido)-N-(methyl- d3)nicotinamide |
| 18 | 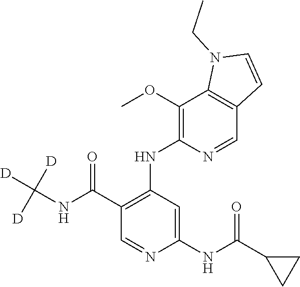 [SMILES: [2H]C([2H])([2H])NC(=O)c1cnc(NC(=O)C2CC2)cc1Nc1ncc2ccn(CC)c2c1OC] | 6-(Cyclopropanecarboxamido)-4- ((1-ethyl-7-methoxy-1H- pyrrolo[3,2-c] pyridin-6-yl) amino)-N-(methyl-d3)nicotinamide |
| 23 | 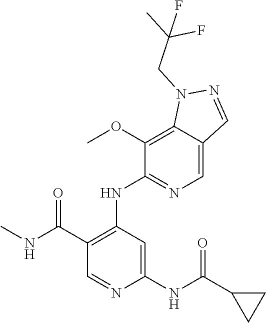 [SMILES: CNC(=O)c1cnc(NC(=O)C2CC2)cc1Nc1ncc2cnn(CC(C)(F)F)c2c1OC] | 6-(Cyclopropanecarboxamido)-4- ((1-(2,2-difluoropropyl)-7- methoxy-1H-pyrazolo[4,3- c]pyridin-6-yl)amino)-N- methylnicotinamide |
| 24 | 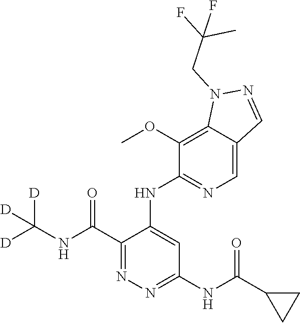 [SMILES: [2H]C([2H])([2H])NC(=O)c1nnc(NC(=O)C2CC2)cc1Nc1ncc2cnn(CC(C)(F)F)c2c1OC] | 6-(Cyclopropanecarboxamido)-4- ((1-(2,2-difluoropropyl)-7- methoxy-1H-pyrazolo[4,3- c]pyridin-6-yl)amino)-N-(methyl- d3)pyridazine-3-carboxamide |
| 28 | 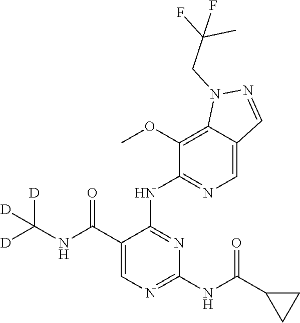 [SMILES: [2H]C([2H])([2H])NC(=O)c1cnc(NC(=O)C2CC2)nc1Nc1ncc2cnn(CC(C)(F)F)c2c1OC] | 2-(cyclopropanecarboxamido)-4- ((1-(2,2-difluoropropyl)-7- methoxy-1H-pyrazolo[4,3- c]pyridin-6-yl)amino)-N-(methyl- d3)pyrimidine-5-carboxamide |
| 30 | 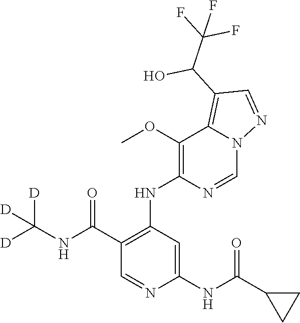 [SMILES: [2H]C([2H])([2H])NC(=O)c1cnc(NC(=O)C2CC2)cc1Nc1ncn2ncc(C(O)C(F)(F)F)c2c1OC] | 6-(cyclopropanecarboxamido)-4- ((4-methoxy-3-(2,2,2-trifluoro-1- hydroxyethyl)pyrazolo[1,5- c]pyrimidin-5-yl)amino)-N- (methyl-d3)nicotinamide |

In yet another aspect, the invention generally relates to a method for preparing a compound disclosed herein, as exemplified by the synthetic schemes and experimental procedure disclosed herein.

In yet another aspect, the invention generally relates to a pharmaceutical composition comprising a compound disclosed herein, effective to treat or reduce one or more diseases or disorders, in a mammal, including a human, and a pharmaceutically acceptable excipient, carrier, or diluent.

In certain embodiments, the pharmaceutical composition is suitable for oral administration.

In certain embodiments, the pharmaceutical composition is suitable for topical administration.

In certain embodiments, the pharmaceutical composition is suitable for GI-restricted administration.

In certain embodiments, the pharmaceutical composition is useful to treat or reduce one or more of inflammatory diseases, immune-mediated diseases and cancers, or a related disease or disorder.

In certain embodiments, the disease or disorder is an inflammatory disease.

In certain embodiments, the disease or disorder is an immune-mediated disease.

In certain embodiments, the disease or disorder is cancer.

In certain embodiments, the disease or disorder is selected from: inflammatory bowel disease, psoriasis, vitiligo, atopic dermatitis, systemic lupus erythematosus, asthma, diabetic nephropathy, chronic myelogenous leukemia (CML), essential thrombocythemia (ET), polycythemia vera (PV), myelofibrosis (MF), breast cancer and ovarian cancer.

In yet another aspect, the invention generally relates to a unit dosage form comprising a pharmaceutical composition disclosed herein.

In certain embodiments, the unit dosage form is a tablet.

In certain embodiments, the unit dosage form is a capsule.

In certain embodiments, the unit dosage form is a topical formulation.

In yet another aspect, the invention generally relates to a method for treating, reducing or preventing a disease or disorder, comprising administering to a subject in need thereof a therapeutically effective amount of a compound having the structural formula of (I):

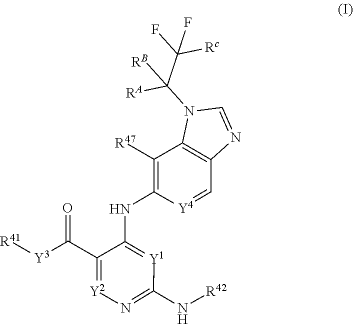

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

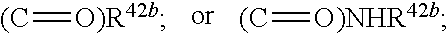

each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a};
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a};
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R⁴¹, C²⁻⁶ alkynyl substituted with 0-3 R^{42a}; and
R⁴⁷ is a C₁₋₃ alkoxy group optionally substituted with 1-3 D or halo,
   wherein the disease or disorder is selected from inflammatory diseases, immune-mediated diseases, cancer, or a related disease or disorder thereof, in a mammal, including a human.

In yet another aspect, the invention generally relates to a method for treating, reducing or preventing a disease or disorder, comprising administering to a subject in need thereof a therapeutically effective amount of a compound having the structural formula of (II):

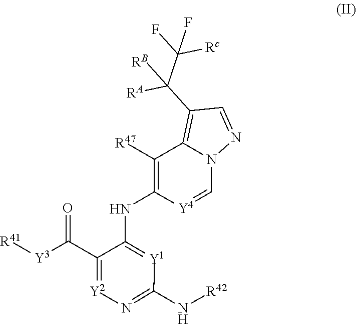

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

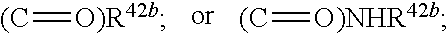

each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42′}, C²⁻⁶ alkynyl substituted with 0-3 R^{42a}; and
R⁴⁷ is a C₁₋₃ alkoxy group optionally substituted with 1-3 D or halo,
   wherein the disease or disorder is selected from inflammatory diseases, immune-mediated diseases, cancer, or a related disease or disorder thereof, in a mammal, including a human.

In yet another aspect, the invention generally relates to a method for treating, reducing or preventing a disease or disorder, comprising administering to a subject in need thereof a therapeutically effective amount of a compound having the structural formula of (III):

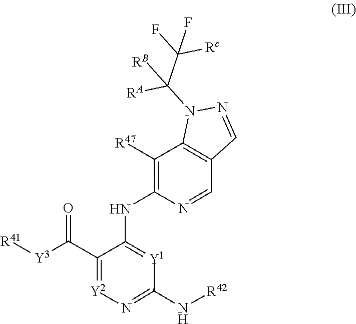

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

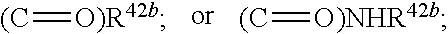

each of R^{A} and R^{B} is independently selected from H, D, halo, OR, and C₁₋₃ alkyl, provided that when both R^{A} and R^{B} is H, R^{C} is not CH₃ or F;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R⁴¹, C²⁻⁶ alkynyl substituted with 0-3 R^{42a}; and
R⁴⁷ is a C₁₋₃ alkoxy group optionally substituted with 1-3 D or halo,
   wherein the disease or disorder is selected from inflammatory diseases, immune-mediated diseases, cancer, or a related disease or disorder thereof, in a mammal, including a human.

In yet another aspect, the invention generally relates to a method for treating, reducing or preventing a disease or disorder, comprising administering to a subject in need thereof a therapeutically effective amount of a compound having the structural formula of (IV):

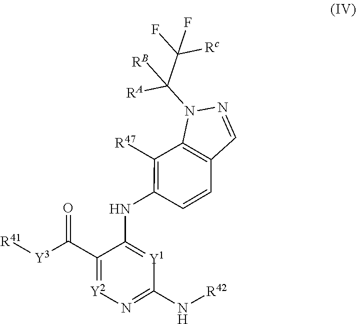

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a}; or

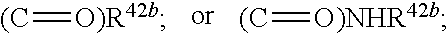

each of R^{A} and R^{B} is independently selected from H, D, halo, OR, and C₁₋₃ alkyl, provided that when both R^{A} and R^{B} is H, R^{C} is not H or F;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42′}, C²⁻⁶ alkynyl substituted with 0-3 R^{42a}; and
R⁴⁷ is a C₁₋₃ alkoxy group optionally substituted with 1-3 D or halo,
   wherein the disease or disorder is selected from inflammatory diseases, immune-mediated diseases, cancer, or a related disease or disorder thereof, in a mammal, including a human.

In yet another aspect, the invention generally relates to a method for treating, reducing or preventing a disease or disorder, comprising administering to a subject in need thereof a therapeutically effective amount of a compound having the structural formula of (V):

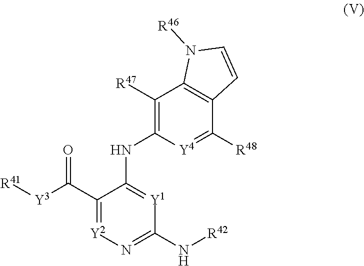

or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
   R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
   an aryl or heteroaryl group, each substituted with 0-2 R^{42a};

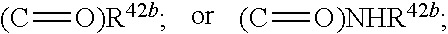

each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c};
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R⁴¹, C²⁻⁶ alkynyl substituted with 0-3 R^{4a};
R⁴⁶ is H, CD₃, a C₁-C₆ alkyl or C₃-C₆ cycloalkyl, with 0-4 of halogen, CN and OR;
R⁴⁷ is a C₁₋₃ alkoxy group optionally substituted with 1-3 D or halo; and
R⁴⁸ is H, halo or a C₁-C₃ alkyl,
   wherein the disease or disorder is selected from inflammatory diseases, immune-mediated diseases, cancer, or a related disease or disorder thereof, in a mammal, including a human.

In certain embodiments, the disease or disorder is an inflammatory disease.

In certain embodiments, the disease or disorder is an immune-mediated disease.

In certain embodiments, the disease or disorder is cancer.

In certain embodiments, the disease or disorder is selected from: inflammatory bowel disease, psoriasis, vitiligo, atopic dermatitis, systemic lupus erythematosus, asthma, diabetic nephropathy, chronic myelogenous leukemia (CML), essential thrombocythemia (ET), polycythemia vera (PV), myelofibrosis (MF), breast cancer and ovarian cancer.

In certain embodiments, administration is via oral administration.
In certain embodiments, administration is via topical administration.
In certain embodiments, administration is via GI-restricted administration.

In yet another aspect, the invention generally relates to use of a compound disclosed herein, and a pharmaceutically acceptable excipient, carrier, or diluent, in preparation of a medicament for treating a disease or disorder.

In certain embodiments of the use, the disease or disorder is one or more of inflammatory diseases, immune-mediated diseases and cancer.

In certain embodiments of the use, the disease or disorder is an inflammatory disease.

In certain embodiments of the use, the disease or disorder is an immune-mediated disease.

In certain embodiments of the use, the disease or disorder is cancer.

In certain embodiments of the use, the medicament is for oral administration.

In certain embodiments of the use, the medicament is for topical administration.

In certain embodiments of the use, the medicament is for GI restriction administration.

As discussed herein, isotope derivative compounds having one or more hydrogen atoms (e.g., 1, 2, 4, 5, 6, 7, 8, 9, 10, etc.) replaced with deuterium atoms are contemplated in the presented invention.

The term “inflammatory disease” refers to a disease or condition characterized by aberrant inflammation, e.g. an increased level of inflammation compared to a control such as a healthy person not suffering from a disease. Examples of inflammatory diseases that may be treated with a compound, pharmaceutical composition, or method described herein include autoimmune diseases, traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis. Such conditions are frequently inextricably intertwined with other diseases, disorders and conditions. A non-limiting list of inflammatory-related diseases, disorders and conditions which may, for example, be caused by inflammatory cytokines, include, arthritis, kidney failure, lupus, asthma, psoriasis, colitis, pancreatitis, allergies, fibrosis, surgical complications (e.g., where inflammatory cytokines prevent healing), anemia, and fibromyalgia. Other diseases and disorders, which may be associated with chronic inflammation include Alzheimer's disease, congestive heart failure, stroke, aortic valve stenosis, arteriosclerosis, osteoporosis, Parkinson's disease, infections, inflammatory bowel disease (IBD), allergic contact dermatitis and other eczemas, systemic sclerosis, transplantation and multiple sclerosis. Some of the aforementioned diseases, disorders and conditions for which a compound of the present disclosure may be particularly efficacious (due to, for example, limitations of current therapies) are described in more detail hereafter.

The term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include acne vulgaris, acute disseminated encephalomyelitis, acute necrotizing hemorrhagic leukoencephalitis, Addison's disease, agammaglobulinemia, Aicardi-Goutibres syndrome (AGS), alopecia areata, alopecia totalis, amyloidosis, ankylosing spondylitis, anti-GBM/anti-TBM nephritis, antiphospholipid syndrome, autoimmune angioedema, autoimmune aplastic anemia, autoimmune dysautonomia, autoimmune hepatitis, autoimmune hyperlipidemia, autoimmune immunodeficiency, autoimmune inner ear disease, autoimmune myocarditis, autoimmune oophoritis, autoimmune pancreatitis, autoimmune retinopathy, autoimmune thrombocytopenic purpura, autoimmune thyroid disease, autoimmune urticaria, axonal or neuronal neuropathies, balo disease, Behcet's disease, bullous pemphigoid, cardiomyopathy, Castleman disease, celiac disease, Chagas disease, chronic atypical neutrophilic dermatosis with lipodystrophy and elevated temperature (CANDLE), chronic active hepatitis, chronic fatigue syndrome, chronic inflammatory demyelinating polyneuropathy, chronic recurrent multifocal ostomyelitis, Churg-Strauss syndrome, cicatricial pemphigoid/benign mucosal pemphigoid, Crohn's disease, Cogans syndrome, cold agglutinin disease, congenital heart block, coxsackie myocarditis, CREST disease, Cushing's disease, demyelinating neuropathies, depression, dermatitis herpetiformis, dermatomyositis, Devic's disease (neuromyelitis optica), discoid lupus, Dressler's syndrome, dry eye syndrome DES (keratoconjunctivitis sicca), endometriosis, eosinophilic esophagitis, eosinophilic fasciitis, erythema nodosum, essential mixed cryoglobulinemia, experimental allergic encephalomyelitis, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, graft-versus-host disease (GVDH), Graves' disease, Guillain-Barre syndrome, Hashimoto's encephalitis, Hashimoto's thyroiditis, hemolytic anemia, Henoch-Schonlein purpura, herpes gestationis, hidradenitis suppurativa, hypogammaglobulinemia, idiopathic thrombocytopenic purpura, IgA nephropathy, IgG4-related sclerosing disease, inflammatory bowel disease (IBD), immunoregulatory lipoproteins, inclusion body myositis, interstitial cystitis, juvenile arthritis, juvenile diabetes (Type 1 diabetes), juvenile dermatomyositis (JDM), juvenile myositis, Kawasaki syndrome, Lambert-Eaton syndrome, leukocytoclastic vasculitis, lichen planus, lichen sclerosus, ligneous conjunctivitis, linear IgA disease, lupus, lyme disease, chronic, Meniere's disease, microscopic polyangiitis, mixed connective tissue disease, Mooren's ulcer, Mucha-Habermann disease, multiple sclerosis (MS), myasthenia gravis, myositis, narcolepsy, neuromyelitis optica, neutropenia, ocular cicatricial pemphigoid, optic neuritis, palindromic rheumatism, pediatric autoimmune neuropsychiatric disorders associated with streptococcus, paraneoplastic cerebellar degeneration, paroxysmal nocturnal hemoglobinuria p, Parry Romberg syndrome, Parsonnage-Turner syndrome, Pars planitis (peripheral uveitis), pemphigus, peripheral neuropathy, perivenous encephalomyelitis, pernicious anemia, POEMS syndrome, polyarteritis nodosa, polycystic ovary syndrome (PCOS), Type I, II, & III autoimmune polyglandular syndromes, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, plaque psoriasis, idiopathic pulmonary fibrosis, pyoderma gangrenosum, pure red cell aplasia, Raynauds phenomenon, reactive Arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, stimulator of interferon genes (STING)-associated vasculopathy with onset during infancy (SAVI), subacute bacterial endocarditis, Susac's syndrome, sympathetic ophthalmia, systemic lupus erythematosus (SLE), Takayasu's arteritis, temporal arteritis/Giant cell arteritis, thrombocytopenic purpura, Tolosa-Hunt syndrome, transplant rejection (allograft transplant rejection), transverse myelitis, Type 1 diabetes, ulcerative colitis, undifferentiated connective tissue disease, uveitis, vasculitis, vesiculobullous dermatosis, vitiligo, or Wegener's granulomatosis.

The term “immune-mediated disease” refers to chronic inflammatory diseases perpetuated by antibodies and cellular immunity. Immune-mediated diseases include, for example, but not limited to, asthma, allergies, arthritis (e.g., rheumatoid arthritis, psoriatic arthritis, and ankylosing spondylitis), juvenile arthritis, inflammatory bowel diseases (e.g., ulcerative colitis and Crohn's disease), endocrinopathies (e.g., type 1 diabetes and Graves' disease), neurodegenerative diseases (e.g., multiple sclerosis (MS)), autistic spectrum disorder, depression, Alzheimer's disease, Guillain-Barre syndrome, obsessive-compulsive disorder, optic neuritis, retinal degeneration, dry eye syndrome DES, Sjogren's syndrome, amyotrophic lateral sclerosis (ALS), Parkinson's disease, Huntington's Disease, Guillain-Barre syndrome, myasthenia gravis, and chronic idiopathic demyelinating disease (CID)), vascular diseases (e.g., autoimmune hearing loss, systemic vasculitis, and atherosclerosis), and skin diseases (e.g., acne vulgaris dermatomyositis, pemphigus, systemic lupus erythematosus (SLE), discoid lupus erthematosus, scleroderma, psoriasis, plaque psoriasis, vasculitics, vitiligo and alopecias). Hashimoto's thyroiditis, pernicious anemia, Cushing's disease, Addison's disease, chronic active hepatitis, polycystic ovary syndrome (PCOS), celiac disease, pemphigus, transplant rejection (allograft transplant rejection), graft-versus-host disease (GVDH).

The term “cancer” as used herein refers to all types of cancer, neoplasm or malignant tumors found in mammals, e.g., humans, including hematological cancers leukemia, and lymphomas, T-ALL, large B-cell lymphoma, solid cancers such as carcinomas and sarcomas. Exemplary cancers include blood cancer, brain cancer, glioma, glioblastoma, neuroblastoma, prostate cancer, colorectal cancer, pancreatic cancer, cervical cancer, gastric cancer, ovarian cancer, lung cancer, and cancer of the head. Exemplary cancers include cancer of the thyroid, endocrine system, brain, breast, cervix, colon, head & neck, liver, kidney, lung, non-small cell lung, melanoma, mesothelioma, ovary, sarcoma, stomach, uterus, medulloblastoma, colorectal cancer, pancreatic cancer. Additional examples include penile, skin—non-melanoma, anal, hepatobiliary, esophagogastric, uterine sarcoma, gastrointestinal stromal tumor, salivary gland, peripheral nervous system, soft tissue sarcoma, bone, renal, myeloproliferative neoplasms, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, metastatic leiomyosarcoma, synovial sarcoma, undifferentiated pleomorphic sarcoma, round cell liposarcoma or prostate cancer.

In certain embodiments of the use, the disease or disorder is selected from: inflammatory bowel disease, psoriasis, vitiligo, atopic dermatitis, systemic lupus erythematosus, asthma, diabetic nephropathy, chronic myelogenous leukemia (CML), essential thrombocythemia (ET), polycythemia vera (PV), myelofibrosis (MF), breast cancer and ovarian cancer.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure. Solvates and polymorphs of the compounds of the invention are also contemplated herein. Solvates of the compounds of the present invention include, for example, hydrates.

Any appropriate route of administration can be employed, for example, parenteral, intravenous, subcutaneous, intramuscular, intraventricular, intracorporeal, intraperitoneal, rectal, or oral administration. Most suitable means of administration for a particular patient will depend on the nature and severity of the disease or condition being treated or the nature of the therapy being used and on the nature of the active compound.

Solid dosage forms for oral administration include capsules, tablets, pills, powders, and granules. In such solid dosage forms, the compounds described herein or derivatives thereof are admixed with at least one inert customary excipient (or carrier) such as sodium citrate or dicalcium phosphate or (i) fillers or extenders, as for example, starches, lactose, sucrose, glucose, mannitol, and silicic acid, (ii) binders, as for example, carboxymethylcellulose, alignates, gelatin, polyvinylpyrrolidone, sucrose, and acacia, (iii) humectants, as for example, glycerol, (iv) disintegrating agents, as for example, agar-agar, calcium carbonate, potato or tapioca starch, alginic acid, certain complex silicates, and sodium carbonate, (v) solution retarders, as for example, paraffin, (vi) absorption accelerators, as for example, quaternary ammonium compounds, (vii) wetting agents, as for example, cetyl alcohol, and glycerol monostearate, (viii) adsorbents, as for example, kaolin and bentonite, and (ix) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

The following examples are meant to be illustrative of the practice of the invention and not limiting in any way.

### EXAMPLES

### Abbreviations

Methanol: MeOH
Dichloromethane: DCM
Petroleum ether: PE
Ethyl acetate: EtOAc/EA
Acetonitrile: ACN or MeCN
Isopropanol: IPA
Triethylamine: TEA
Tetrahydrofuran: THF
Sodium hydroxide: NaOH
Propylphosphonic Acid Anhydride: T₃P
Nitrogen: N₂
N,N-Diisopropylethylamine: DIPEA
N, N-Dimethylformamide: DMF
4-Methylbenzenesulfonic acid: pTSA or TsOH
N,N-Dimethylformamide dimethyl acetal: DMF-DMA
N-Iodosuccinimide: NIS
Lithium bis(trimethylsilyl)amide: LiHMDS
2-(Trimethylsilyl)ethoxymethyl chloride: SEMCl
2,4-Dimethoxybenzylamine: DMBNH₂
4-Methoxybenzylamine: PMBNH₂
2,2,6,6-Tetramethylpiperidinooxy: TEMPO
Thin-Layer Chromatography: TLC
High Performance Liquid Chromatography: HPLC
Room temperature: r.t.
Hours: h

Representative methods of prep-HPLC: Flow rate and gradient may change.

Exemplary methods for prep-HPLC are provided below.

Method A: NH₄HCO₃: Column: Gilson2-Xbridge C18 19*150 mm, 5 μm; mobile phase: CH₃CN in water (0.1% NH₄HCO₃) from 20% to 60%, flow rate: 15 mL/min.

Method B: TFA: Column: Waters-Xbridge C18 10*190 mm, 5 μm; mobile phase: CH₃CN in water (0.1% TFA) from 15% to 40%, flow rate: 15 mL/min.

Method C: HCOOH: Column: Waters-Xbridge C18 10*190 mm, 5 μm; mobile phase: CH₃CN in water (0.1% formic acid) from 15% to 40%, flow rate: 15 mL/min.

Method D: HCOOH: Column: Waters SunFire® Prep C18 OBD™ (5 micron, 19*150 mm); mobile phase: CH₃CN in water (0.1% formic acid) from 18% to 38%, flow rate: 20 mL/min.

Method E: NH₄HCO₃: Column: Waters Xbridge @Prep C18 OBD™ (5 micron, 19*150 mm); mobile phase: CH₃CN in water (10 mM NH₄HCO₃) from 20% to 60%, flow rate: 20 mL/min.

Method F: HCOOH: Columns: Waters Atlantis T3 19*150 mm, 5 μm; mobile phase: CH₃CN in H₂O (0.1% formic acid) from 16% to 30%, flow rate: 20 mL/min.

### Representative Methods of Chiral Prep-HPLC:

Method G: Gilson 281, Daicel Chiralpak IB N, 10 μm 30*250 mm; Mobile phase: Hexane/EtOH=60/40, Flow rate: 25 mL/min.

Method H: Gilson 281, Daicel Chiralpak IB N, 10 μm 30*250 mm; Mobile phase: Hexane/EtOH=50/50, Flow rate: 25 mL/min.

### Representative Methods of Analytical-HPLC:

Method 1: Analysis was performed on an Agilent 1200_series HPLC-6120MS. UHPLC Long Gradient Equivalent 5% to 95% acetonitrile in water (containing 0.02% NH₄OAc) run time of 6.5 minutes with a flow rate of 1.5 mL/min. A Waters Xbridge C18 column (18.5 micron, 4.6*50 mm) was used at a temperature of 40° C.

Method 2: Analysis was performed on an Agilent 1200_series HPLC-6120MS. UHPLC Long Gradient Equivalent 5% to 95% acetonitrile in water (containing 0.1% trifluoroacetic acid) run time of 6.5 minutes with a flow rate of 1.5 mL/min. A Waters Xbridge C18 column (18.5 micron, 4.6*50 mm) was used at a temperature of 40° C.

Method 3: Analysis was performed on an Agilent 1260_series HPLC-6120MS. UHPLC Long Gradient Equivalent 5% to 95% acetonitrile in water (containing 0.02% NH₄OAc) run time of 2.5 minutes with a flow rate of 0.5 mL/min. A diamonsil Plus C18 column (18.5 micron, 4.6*30 mm) was used at a temperature of 40° C.

Method 4: Analysis was performed on an Agilent 1260_series HPLC-6125C MS. HPLC Long Gradient Equivalent 20% to 100% acetonitrile in water (containing 0.1% formic acid) run time of 6 minutes with a flow rate of 0.8 mL/min. Agilent ZORBAX SB—C18 column (1.8 micron, 2.1*50 mm) was used at a temperature of 30° C.

Method 5: Analysis was performed on a SHIMADZU 20A HPLC. HPLC Long Gradient Equivalent Hexane/EtOH (60/40) run time of 20 minutes with a flow rate of 1 mL/min. CHIRALPAK IB N-5 (5 um, 4.6*250 mm) was used at a temperature of 30° C.

Method 6: Analysis was performed on a SHIMADZU 20A HPLC. HPLC Long Gradient Equivalent Hexane/EtOH (50/50) run time of 20 minutes with a flow rate of 1 mL/min. CHIRALPAK IB N-5 (5 um, 4.6*250 mm) was used at a temperature of 30° C.

### Intermediate A

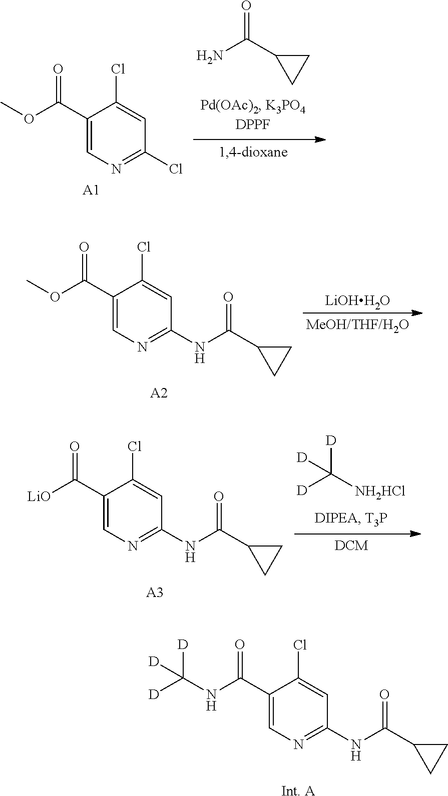

### Step 1. Methyl 4-chloro-6-(cyclopropanecarboxamido) nicotinate (A2)

A mixture of A1 (2.0 g, 9.71 mmol), cyclopropanecarboxamide (826 mg, 9.71 mmol), Pd(OAc)₂ (109 mg, 0.49 mmol), dppf (538 mg, 0.97 mmol), K₃PO₄ (4.12 g, 19.42 mmol) in dioxane (30 mL) was stirred at 90° C. for 4 h under N₂. The mixture was diluted with H₂O (100 mL), extracted with EtOAc (30 mL*3), washed with brine (30 mL), dried over Na₂SO₄, concentrated and purified by flash chromography (PE/EA=10/1 to 1/1) to get compound A2 (1.8 g, 73% yield) as a white solid. LC-MS (ESI, Method 4) t_{R}=3.33 min, m/z (M+H)⁺=255.0.

### Step 2. Lithium 4-chloro-6-(cyclopropanecarboxamido)nicotinate (A3)

To a solution of A2 (500 mg, 1.96 mmol) in a co-solvent of MeOH (2 mL), THF (2 mL) and water (1 mL) was added LiOH·H₂O (165 mg, 3.93 mmol). Then the mixture was stirred at r.t. overnight. The mixture was concentrated to dryness to give compound A3 (480 mg, 99% yield) as a white solid. LC-MS (ESI, Method 4) t_{R}=3.81 min, m/z (M+H)⁺=241.1.

### Step 3. 4-Chloro-6-(cyclopropanecarboxamido)-N-(methyl-d₃)nicotinamide (Int. A)

To a solution of A3 (480 mg, 1.95 mmol) in DCM (15 mL) was sequentially added methan-di-amine hydrochloride (275 mg, 3.89 mmol), DIPEA (1.51 g, 11.68 mmol) and T₃P (1.86 g, 2.92 mmol, 50% in EtOAc) at 0° C. The resulting mixture was stirred at r.t. overnight. The mixture was diluted with H₂O (30 mL) and extracted with DCM (30 mL*3). The organic layer was washed with brine (50 mL), dried over Na₂SO₄ and filtered. The filtrate was concentrated to dryness to give Int. A (300 mg, 60% yield) as a white solid. LC-MS (ESI, Method 4) t_{R}=2.25 min, m/z (M+H)⁺=257.1.

### Intermediate B

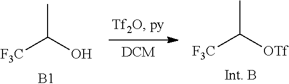

### Step 1. 1,1,1-Trifluoropropan-2-yl trifluoromethanesulfonate (Int. B)

To a solution of B1 (1.24 g, 10.87 mmol) and pyridine (1.12 g, 14.13 mmol, 1.14 mL) in DCM (10 mL) was added trifluoromethanesulfonic anhydride (3.37 g, 11.96 mmol, 2.01 mL) at 0° C., then the mixture was stirred at r.t. for 2 h. The mixture was filtered, The filter cake was rinsed with DCM (17 mL) to yield the compound Int. B (27 mL, 10.87 mmol, 0.4 M in DCM, yield given) as a solution, which was directly used in the next step.

### Intermediate C

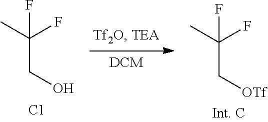

### Step 1. 2,2-Difluoropropyl trifluoromethanesulfonate (Int. C)

To a solution of C1 (1 g, 10.4 mmol) and triethylamine (1.5 g, 15.6 mmol) in DCM (15 mL) was added trifluoromethanesulfonic anhydride (4.4 g, 15.6 mmol) dropwise at −20° C. The reaction was stirred at −20° C. for 16 h and diluted with DCM (10 mL). The mixture was washed with ice water (10 mL). The separated organic layer was washed with 20% Na₂CO₃ aqueous solution (10 mL) and brine (10 mL), dried over Na₂SO₄, filtered and concentrated in vacuo to afford Int. C (2.3 g, 97% yield) as a black oil. ¹H NMR (400 MHz, DMSO-d₆) δ 5.08 (t, J=13.6 Hz, 2H), 1.72 (t, J=19.2 Hz, 3H).

### Intermediate D

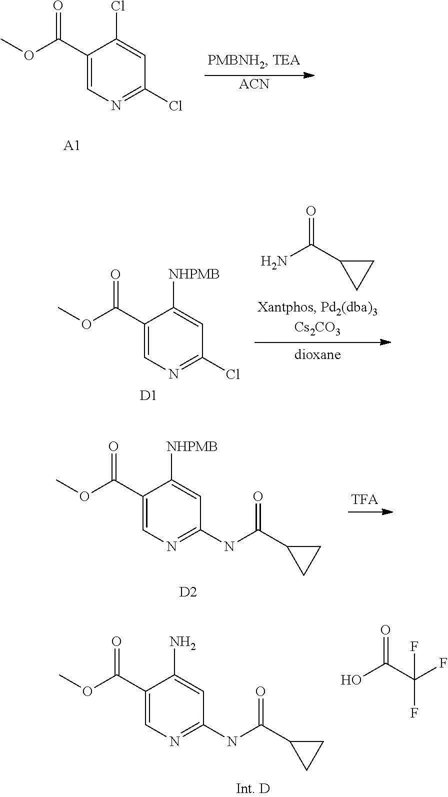

### Step 1. Methyl 6-chloro-4-((4-methoxybenzyl)amino)nicotinate (D1)

To a solution of A1 (5 g, 24.27 mmol) in ACN (8 mL) was added (4-methoxyphenyl)methanamine (3.33 g, 24.27 mmol, 3.2 mL) and TEA (4.91 g, 48.54 mmol, 6.8 mL). Then the mixture was stirred at r.t. for 24 h. The mixture was diluted with H₂O (100 mL), extracted with EA (50 mL*3), washed with brine, dried over Na₂SO₄, concentrated and purified by flash chromatography (PE/EA=20/1 to 5/1) to get compound D1 (6.5 g, 87% yield) as an off-white solid. LC-MS (ESI, Method 4) t_{R}=4.18 min, m/z (M+H)⁺=307.1.

### Step 2. Methyl 6-(cyclopropanecarboxamido)-4-((4-methoxybenzyl)amino)nicotinate (D2)

A mixture of D1 (2 g, 6.52 mmol), cyclopropanecarboxamide (1.11 g, 13.04 mmol), XantPhos (754 mg, 1.30 mmol), Pd₂(dba)₃ (597 mg, 0.65 mmol), Cs₂CO₃ (5.31 g, 16.30 mmol) in 1,4-dioxane (30 mL) was stirred at 110° C. for 2 h. Then the mixture was diluted with H₂O (100 mL), extracted with EA (60 mL*3), washed with brine, dried over Na₂SO₄ and concentrated to get compound D2 (2.3 g, 99% yield) as a yellow solid. LC-MS (ESI, Method 4) t_{R}=2.91 min, m/z (M+H)⁺=356.2.

### Step 3. Methyl 4-amino-6-(cyclopropanecarboxamido)nicotinate 2,2,2-trifluoroacetate (Int. D)

A solution of D2 (2.1 g, 5.91 mmol) in TFA (10 mL) was stirred at 80° C. for 16 h. Then the mixture was concentrated and diluted with EA (10 mL), filtered and wash with EA (5 mL*2). Then the solid was dried to get compound Int. D (1.8 g, 87% yield, TFA salt) as an off-white solid. LC-MS (ESI, Method 4) t_{R}=1.28 min, m/z (M+H)⁺=236.2.

### Intermediate E

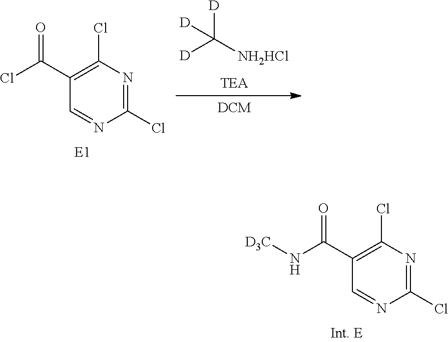

### Step 1. 2,4-Dichloro-N-(methyl-d₃)pyrimidine-5-carboxamide (Int. E)

To a mixture of methan-d₃-amine hydrochloride (1.40 g, 19.86 mmol) in DCM (200 mL) was added E1 (3.5 g, 16.55 mmol) slowly followed by TEA (1.68 g, 16.55 mmol, 2.31 mL) at −78° C. After stirring for 1 h at this temperature, the reaction was quenched with water (30 mL). The organic layer was separated and concentrated. The residue was purified by flash chromatography on silica gel (PE/EA=5/1) to afford Int. E (1.38 g, 35% yield) as a white solid. ¹H NMR (400 MHz, DMSO-d₆) δ 8.88 (s, 1H), 8.85 (s, 1H). LC-MS (ESI, Method 3) t_{R}=0.80 min, m/z (M+H)⁺=208.9.

### Intermediate F

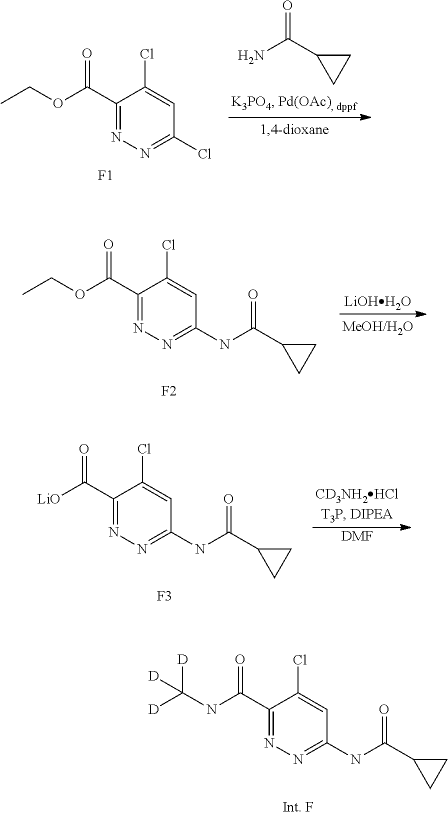

### Step 1. Methyl 4-chloro-6-(cyclopropanecarboxamido)pyridazine-3-carboxylate (F2)

To a solution of F1 (500 mg, 2.4 mmol) and cyclopropanecarboxamide (408 mg, 4.8 mmol) in 1,4-dioxane (20 mL) was added Pd(OAc)₂(54 mg, 0.24 mmol), dppf (265 mg, 0.48 mmol) and K₃PO₄(1.01 g, 4.8 mmol), then the mixture was stirred at 90° C. for 4 h under N₂ atmosphere. The mixture was diluted with H₂O (50 mL), extracted with EtOAc (30 mL*3), washed with brine (30 mL), dried over Na₂SO₄, concentrated and purified by flash chromatography (DCM/MeOH=100/1 to 10/1) to get compound F2 (200 mg, 33% yield) as an off-white solid. LC-MS (ESI, Method 4) t_{R}=3.46 min, m/z (M+H)⁺=270.1.

### Step 2. Lithium 4-chloro-6-(cyclopropanecarboxamido)pyridazine-3-carboxylate (F3)

To a solution of F2 (100 mg, 0.41 mmol) in MeOH (1 mL) and H₂O (1 mL) was added LiOH·H₂O (34 mg, 0.82 mmol), then the mixture was stirred at r.t. for 2 h. The reaction mixture was concentrated to get F3 (132 mg, crude) and used for the next step directly without further purification. LC-MS (ESI, Method 4) t_{R}=1.44 min, m/z (M+H)⁺=242.1.

### Step 3. 4-Chloro-6-(cyclopropanecarboxamido)-N-(methyl-d₃)pyridazine-3-carboxamide (Int. F)

To a solution of F3 (132 mg, crude) in DMF (1 mL) was added methan-d₃-amine hydrochloride (58 mg, 0.82 mmol) and DIPEA (251 mg, 1.95 mmol), the mixture was stirred at r.t. for 20 min, then T₃P (368 mg, 0.58 mmol, 50% in EtOAc) was added to the reaction mixture, the reaction mixture was stirred at r.t. for 3 h. The mixture was diluted with H₂O (50 mL), extracted with EtOAc (30 mL*3), washed with brine (30 mL), dried over Na₂SO₄, concentrated and purified by flash chromatography (DCM/MeOH=100/1 to 10/1) to get compound Int. F (15 mg, 14% yield, over two steps) as an off-white solid. LC-MS (ESI, Method 4) t_{R}=2.24 min, m/z (M+H)⁺=258.1.

### Intermediate G

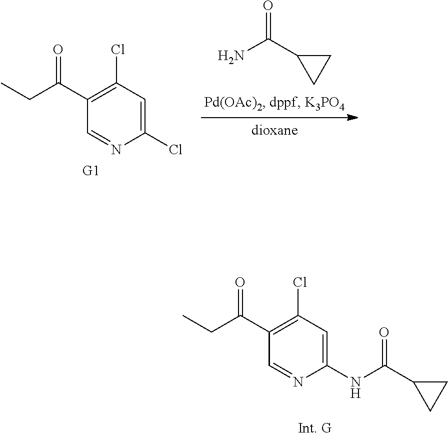 [SMILES: CCC(=O)c1cnc(Cl)cc1Cl ; NC(=O)C1CC1 ; CCC(=O)c1cnc(NC(=O)C2CC2)cc1Cl]

### Step 1. N-(4-chloro-5-propionylpyridin-2-yl)cyclopropanecarboxamide (Int. G)

A mixture of 1-(4,6-dichloropyridin-3-yl)propan-1-one (170 mg, 0.83 mmol, CAS 66525-49-1), cyclopropanecarboxamide (85 mg, 0.99 mmol), dppf (23 mg, 0.042 mmol), Pd(OAc)₂ (19 mg, 0.042 mmol) and K₃PO₄ (265 mg, 1.25 mmol) in dioxane (3 mL) was stirred at 90° C. for 3 h under N₂ atmosphere. The reaction mixture was concentrated and the residue was purified by flash chromatography on silica gel (PE/EA=3/1) to afford Int. G (140 mg, 67% yield) as a white solid. ¹H NMR (400 MHz, CDCl₃) δ 8.49 (s, 1H), 8.34 (s, 1H), 8.30 (brs, 1H), 2.99 (q, J=7.2 Hz, 2H), 1.60-1.53 (m, 1H), 1.21 (t, J=7.2 Hz, 3H), 1.16-1.12 (m, 2H), 0.98-0.93 (m, 2H).

### Example 1

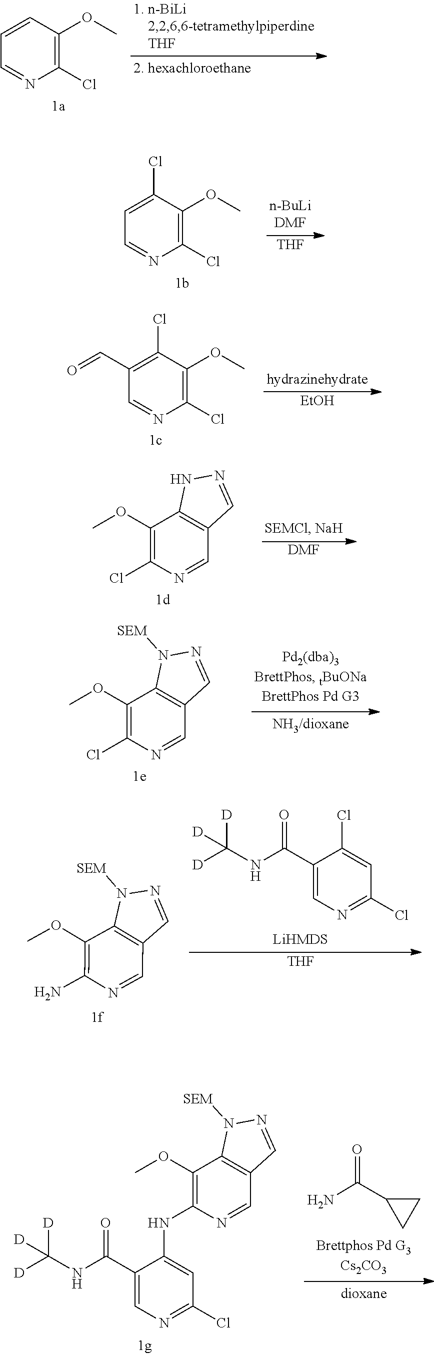

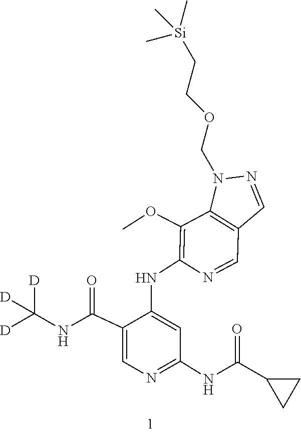 [SMILES: [2H]C([2H])([2H])NC(=O)c1cnc(NC(=O)C2CC2)cc1Nc1ncc2cnn(COCC[Si](C)(C)C)c2c1OC]

### Step 1. 2,4-Dichloro-3-methoxypyridine (1b)

To a solution of n-BuLi (5.5 mL, 13.93 mmol, 2.5 M in THF) in THF (25 mL) was added 2,2,6,6-tetramethylpiperidine (2.3 g, 16.72 mmol) at −78° C. The reaction mixture was stirred at −78° C. for 15 min. Then to the reaction mixture was added a solution of 1a (2.0 g, 13.93 mmol) in THF (25 mL). The reaction mixture was stirred at −78° C. for 4 h. A solution of hexachloroethane (6.6 g, 27.86 mmol) in THF (50 mL) was added dropwise to the reaction and the temperature was kept below −60° C. After stirring for another 2 h at −78° C., the reaction mixture was quenched with sat. NH₄Cl (20 mL), diluted with water (40 mL) and extracted with EtOAc (30 mL*3). The organic layer was washed with brine (20 mL), dried over Na₂SO₄, filtered and concentrated. The residue was purified by flash chromatography on silica gel (PE/EA=10/1) to afford compound 1b (700 mg, 28% yield) as a yellow oil. ¹H NMR (400 MHz, CDCl₃) δ 8.06 (d, J=5.2 Hz, 1H), 7.29 (d, J=5.6 Hz, 1H), 3.95 (s, 3H).

### Step 2. 4,6-Dichloro-5-methoxynicotinaldehyde (1c)

To a solution of n-BuLi (9.4 mL, 23.59 mmol, 2.5 M in THF) in THF (30 mL) was added 1b (2.8 g, 15.73 mmol) at −78° C. After stirring at −78° C. for 30 min, to the reaction mixture was added anhydrous DMF (1.7 g, 23.59 mmol) at −78° C. After stirring for 2 h at −78° C., the reaction was quenched with sat. NH₄Cl (20 mL) and extracted with EtOAc (100 mL). The organic layer was washed with brine (20 mL), dried over Na₂SO₄, filtered and concentrated. The residue was purified by flash chromatography on silica gel (PE/EA=5/1) to afford compound 1c (2.0 g, 61% yield) as a white solid. ¹H NMR (400 MHz, DMSO-d₆) δ 10.25 (s, 1H), 8.60 (s, 1H), 3.93 (s, 3H).

### Step 3. 6-Chloro-7-methoxy-1H-pyrazolo[4,3-c]pyridine (1d)

A mixture of 1c (1.5 g, 7.28 mmol) and hydrazine hydrate (3.6 g, 21.84 mmol, 30% wt in water) in EtOH (20 mL) was stirred at 100° C. for 8 h. The reaction mixture was concentrated. The residue was purified by flash chromatography on silica gel (PE/EA=3/1) to afford compound 1d (300 mg, 22% yield) as a white solid. ¹H NMR (400 MHz, DMSO-d₆) δ 13.95 (s, 1H), 8.67 (s, 1H), 8.37 (s, 1H), 4.04 (s, 3H).

### Step 4. 6-Chloro-7-methoxy-1-((2-(trimethylsilyl)ethoxy)methyl)-1H-pyrazolo[4,3-c]pyridine (1e)

To a mixture of 1d (3 g, 16.34 mmol) in DMF (50 mL) was added NaH (1.3 g, 32.68 mmol, 60% in mineral oil) at 0° C. The mixture was stirred at 0° C. for 30 min and to it was added SEMCl (4 g, 24.51 mmol) at 0° C. After stirring at 10° C. for 1 h, the reaction was quenched with H₂O (50 mL) and extracted with EtOAc (50 mL). The separated organic layer was washed with brine (50 mL), dried over Na₂SO₄ and filtered. The filtrate was concentrated. The residue was purified by flash chromatography on silica gel (PE/EA=3/1) to afford 1e (1.7 g, 33% yield) as a yellow oil. LC-MS (ESI, Method 3) t_{R}=1.88 min, m/z (M+H)⁺=314.1.

### Step 5. 7-Methoxy-1-((2-(trimethylsilyl)ethoxy)methyl)-1H-pyrazolo[4,3-c]pyridin-6-amine (1f)

A mixture of 1e (1.7 g, 5.42 mmol), Pd₂(dba)₃ (248 mg, 0.27 mmol), BrettPhos (145 mg, 0.27 mmol), ^{t}BuONa (780 mg, 8.12 mmol) and sat. NH₃/dioxane (20 mL) was stirred at 100° C. for 12 h under N₂ atmosphere. The mixture was cooled and concentrated. The residue was purified by prep-HPLC (Method A) to afford if (550 mg, 34% yield) as a white solid. LC-MS (ESI, Method 3) t_{R}=1.69 min, m/z (M+H)⁺=295.2.

### Step 6. 6-Chloro-4-((7-methoxy-1-((2-(trimethylsilyl)ethoxy)methyl)-1H-pyrazolo[4,3-c]pyridin-6-yl)amino)-N-(methyl-d₃)nicotinamide (1g)

To a solution of if (500 mg, 1.70 mmol) and 4,6-dichloro-N-(methyl-d₃)nicotinamide (389 mg, 1.87 mmol) in THF (5 mL) was added LiHMDS (6.79 mL, 6.79 mmol, 1 M in THF) at −10° C. The reaction was stirred at −10° C. to r.t. for 1 h and quenched with ice-water (20 mL). The resultant mixture was extracted with EtOAc (30 mL*2) and the combined organic layer was concentrated. The residue was purified by flash chromatography on silica gel (DCM/MeOH=97/3) to afford 1g (610 mg, 77% yield) as a brown solid. LC-MS (ESI, Method 3) t_{R}=1.45 min, m/z (M+H)⁺=466.3.

### Step 7. 6-(Cyclopropanecarboxamido)-4-((7-methoxy-1-((2-(trimethylsilyl)ethoxy)methyl)-1H-pyrazolo[4,3-c]pyridin-6-yl)amino)-N-(methyl-d₃)nicotinamide (1)

A mixture of 1g (600 mg, 1.29 mmol), cyclopropanecarboxamide (548 mg, 6.44 mmol), BrettPhos Pd G3 (233 mg, 0.26 mmol) and Cs₂CO₃ (839 mg, 2.57 mmol) in 1,4-dioxane (6 mL) was stirred at 90° C. for 12 h under N₂ atmosphere. The reaction mixture was concentrated and the residue was purified by flash chromatography on silica gel (DCM/MeOH=20/1) to afford 1 (200 mg, 30% yield) as a white solid. ¹H NMR (400 MHz, DMSO-d₆) δ 11.79 (s, 1H), 10.79 (s, 1H), 9.37 (s, 1H), 8.76 (s, 1H), 8.73 (s, 1H), 8.65 (s, 1H), 8.38 (s, 1H), 5.86 (s, 2H), 4.00 (s, 3H), 3.69 (t, J=8.4 Hz, 2H), 2.12-2.07 (m, 1H), 0.97-0.87 (m, 6H), 0.03 (s, 9H). LC-MS (ESI, Method 2) t_{R}=4.13 min, m/z (M+H)⁺=515.1.

### Example 2

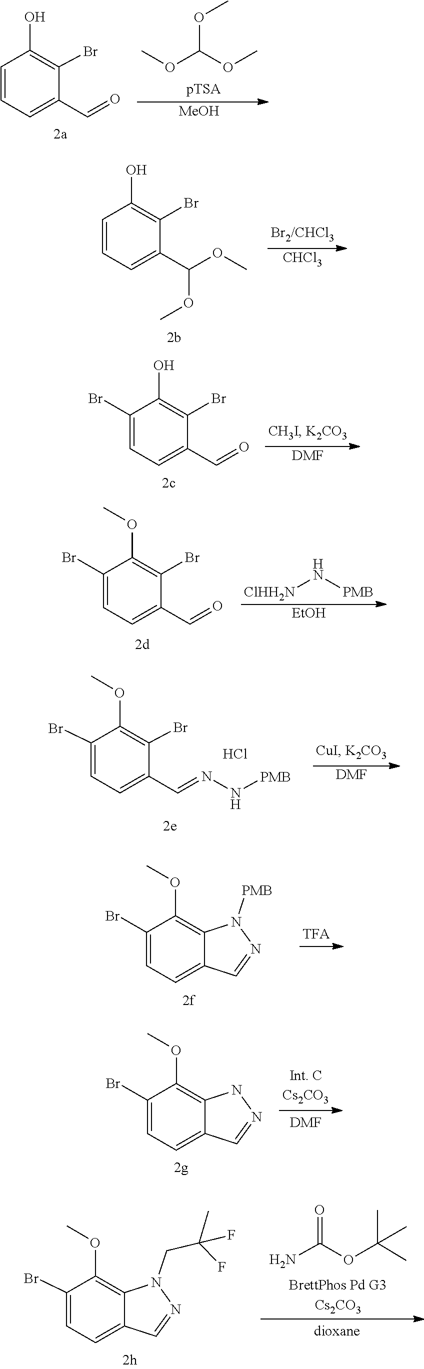

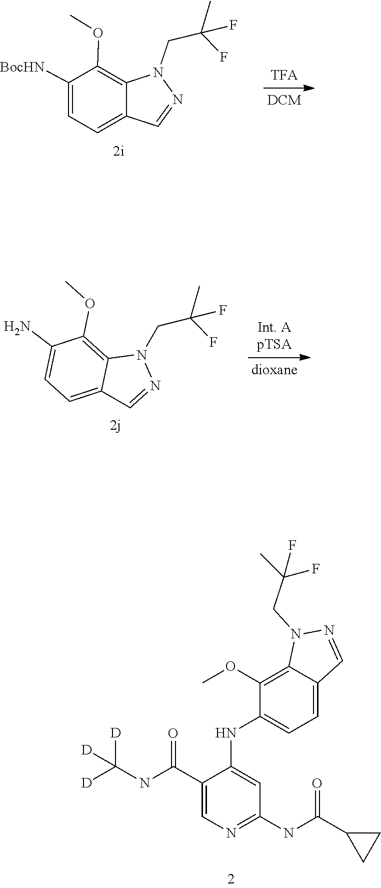

### Step 1. 2-Bromo-3-(dimethoxymethyl)phenol (2b)

To a solution of 2a (2 g, 9.95 mmol) and trimethoxymethane (5.28 g, 49.75 mmol, 5.45 mL) in MeOH (30 mL) was added pTSA (172 mg, 1.00 mmol). The mixture was stirred at 100° C. for 16 h. The solvent was removed under vacuum to give crude product 2b (2.5 g, yield given) as a yellow oil. LC-MS (ESI, Method 4) t_{R}=3.54 min/z (⁷⁹Br, M+H−32)⁺=215.0.

### Step 2. 2,4-Dibromo-3-hydroxybenzaldehyde (2c)

To a solution of 2b (500 mg, 2.02 mmol) in CHCl₃ (5 mL) was added a solution of molecular bromine (323 mg, 2.02 mmol) in CHCl₃ (5 mL) at 0° C. The reaction was stirred at 25° C. for 16 h. The reaction was quenched by aq. Na₂S₂O₃ (40 mL) and extracted with EtOAc (25 mL*3). The combined organic layer was washed with brine (25 mL), dried over Na₂SO₄, filtered and concentrated. The residue was purified by flash chromatography on silica gel (PE/EA=10/1 to 3/1) to give the title compound 2c (300 mg, 53% yield) as a white solid. ¹H NMR (400 MHz, DMSO-d₆) δ 10.44 (s, 1H), 10.17 (d, J=0.8 Hz, 1H), 7.83-7.65 (m, 1H), 7.27 (d, J=8.4 Hz, 1H). LC-MS (ESI, Method 4) t_{R}=2.92 min, m/z (M+H)⁺=280.9.

### Step 3. 2,4-Dibromo-3-methoxybenzaldehyde (2d)

To a solution of 2c (300 mg, 1.07 mmol) and K₂CO₃ (296 mg, 2.14 mmol) in DMF (3 mL) was added iodomethane (228 mg, 1.61 mmol). The mixture was stirred at 25° C. for 2 h, then poured into water (20 mL) and extracted with EtOAc (20 mL*3). The combined organic layer was washed with brine (30 mL), dried over Na₂SO₄, filtered and concentrated under vacuum to give the title product 2d (300 mg, 95% yield) as a yellow solid. LC-MS (ESI, Method 4) t_{R}=4.00 min, m/z (M+H)⁺=294.9.

### Step 4. (E)-1-(2,4-dibromo-3-methoxybenzylidene)-2-(4-methoxybenzyl)hydrazine hydrochloride (2e)

Compound 2d (3.0 g, 10.21 mmol) and (4-methoxybenzyl)hydrazine hydrochloride (2.12 g, 11.23 mmol) were dissolved in EtOH (30 mL). The resulting mixture was stirred at 25° C. for 16 h and then cooled to 0° C. The cloudy mixture was filtered and washed with EtOH (5 mL) to afford the title compound 2e (3.5 g, 74% yield) as a pale yellow solid. LC-MS (ESI, Method 4) t_{R}=5.22 min, m/z (M+H)⁺=427.0.

### Step 5. 6-Bromo-7-methoxy-1-(4-methoxybenzyl)-1H-indazole (2f)

To a solution of 2e (3.5 g, 7.53 mmol) in DMF (50 mL) was added K₂CO₃ (2.6 g, 18.83 mmol) and CuI (143 mg, 0.75 mmol). The mixture was stirred at 100° C. for 16 h. Water (200 mL) was added to above mixture. The solution was extracted with EtOAc (60 mL*3). The combined organic layer was washed with brine (100 mL), dried over Na₂SO₄, filtered and concentrated under vacuum to give crude compound 2f (2.4 g, 91% yield) as a yellow oil. LC-MS (ESI, Method 4) t_{R}=4.81 min, m/z (1Br, M+H)⁺=347.1.

### Step 6. 6-Bromo-7-methoxy-1H-indazole (2g)

2f (2.4 g, 6.91 mmol) was dissolved in TFA (20 mL), then the mixture was stirred at 90° C. for 4 h. Then the mixture was concentrated and diluted with H₂O (50 mL), adjusted pH to 7 with aq. NaHCO₃, then extracted with EtOAc (50 mL*3), washed with brine (50 mL), dried over Na₂SO₄, concentrated and purified by flash chromatography (PE/EA=1/1 to 1/10) to get the compound 2g (1.4 g, 89% yield) as a yellow solid. LC-MS (ESI, Method 4) t_{R}=3.39 min, m/z (⁷⁹Br, M+H)⁺=226.9.

### Step 7. 6-Bromo-1-(2,2-difluoropropyl)-7-methoxy-1H-indazole (2h)

To a solution of 2g (400 mg, 1.76 mmol) in DMF (5 mL) was added Cs₂CO₃ (1.15 g, 3.52 mmol) and Int. C (804 mg, 3.52 mmol). Then the mixture was stirred at r.t. for 2 h. The mixture was diluted with H₂O (20 mL), extracted with EtOAc (20 mL*3), washed with brine, dried over Na₂SO₄, concentrated and purified by flash chromatography (PE/EA=20/1 to 1/1) to get the compound 2h (100 mg, 19% yield) as a yellow oil. ¹H NMR (400 MHz, CDCl₃) δ 8.03 (s, 1H), 7.34 (d, J=6.8 Hz, 1H), 7.27 (d, J=6.8 Hz, 1H), 4.95 (t, J=13.2 Hz, 2H), 4.05 (s, 3H), 1.66 (t, J=15.2 Hz, 3H). LC-MS (ESI, Method 4) t_{R}=3.16 min, m/z (⁷⁹Br, M+H)⁺=305.1.

### Step 8. Tert-butyl (1-(2,2-difluoropropyl)-7-methoxy-1H-indazol-6-yl)carbamate (2i)

A mixture of 2h (100 mg, 0.33 mmol), tert-butyl carbamate (77 mg, 0.66 mmol), Cs₂CO₃ (267 mg, 0.82 mmol), BrettPhos Pd G3 (30 mg, 0.033 mmol) in dioxane (2 mL) was stirred at 100° C. for 16 h. Then the mixture was concentrated, diluted with EtOAc, filtered and the filtrate was concentrated to get the crude compound 2i (110 mg, 98% yield) as a yellow solid. LC-MS (ESI, Method 4) t_{R}=3.14 min, m/z (M+H)⁺=342.2.

### Step 9. 1-(2,2-Difluoropropyl)-7-methoxy-1H-indazol-6-amine (2j)

To a solution of 2i (110 mg, 0.32 mmol) in DCM (2 mL) was added TFA (1 mL) at r.t., then the mixture was concentrated and diluted with H₂O (20 mL), basified to pH >7 with aq. Na₂CO₃, extracted with EtOAc (20 mL*3). The combined organic layer was washed with brine (20 mL), dried over Na₂SO₄, concentrated and purified by flash chromatography (EA in PE is 0% to 45%) to get compound 2j (70 mg, 90% yield) as a yellow oil. LC-MS (ESI, Method 4) t_{R}=2.05 min, m/z (M+H)⁺=242.1.

### Step 4. 6-(Cyclopropanecarboxamido)-4-((1-(2,2-difluoropropyl)-7-methoxy-1H-indazol-6-yl)amino)-N-(methyl-d₃)nicotinamide (2)

To a solution of 2j (40 mg, 0.17 mmol) and Int. A (47 mg, 0.18 mmol) in dioxane (1.5 mL) was added pTSA (29 mg, 0.17 mmol), then the mixture was stirred at r.t. for 16 h. The mixture was concentrated and purified by prep-HPLC (Method E) to get compound 2 (43.3 mg, 57% yield) as a pale yellow solid. ¹H NMR (400 MHz, DMSO-d₆) δ 10.73 (s, 1H), 10.55 (s, 1H), 8.63 (s, 1H), 8.53 (s, 1H), 8.15 (s, 1H), 7.83 (s, 1H), 7.54 (d, J=8.4 Hz, 1H), 7.15 (d, J=8.4 Hz, 1H), 4.97 (t, J=13.2 Hz, 2H), 3.78 (s, 3H), 1.96-1.91 (m, 1H), 1.65 (t, J=19.2 Hz, 3H), 0.74-0.71 (m, 4H). LC-MS (ESI, Method 4) t_{R}=2.12 min, m/z (M+H)⁺=462.3.

### Example 3

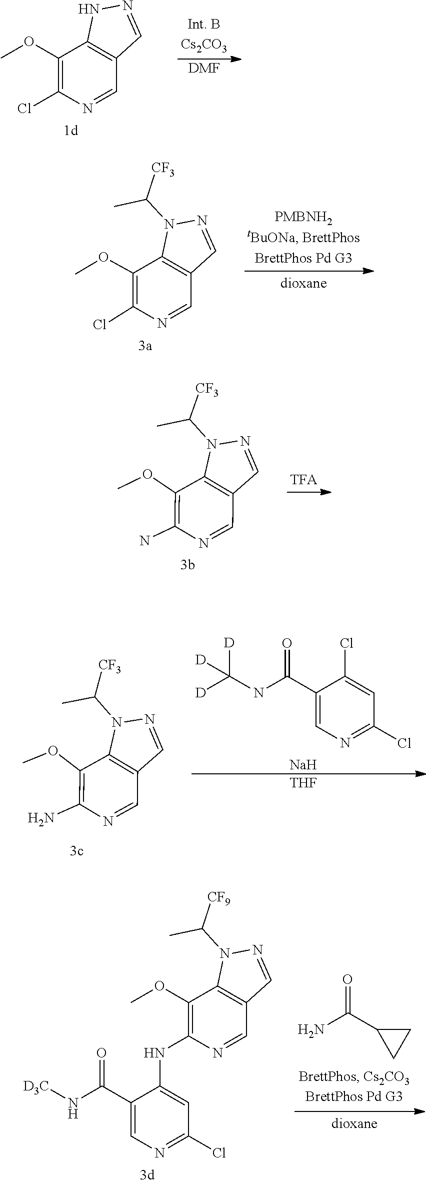

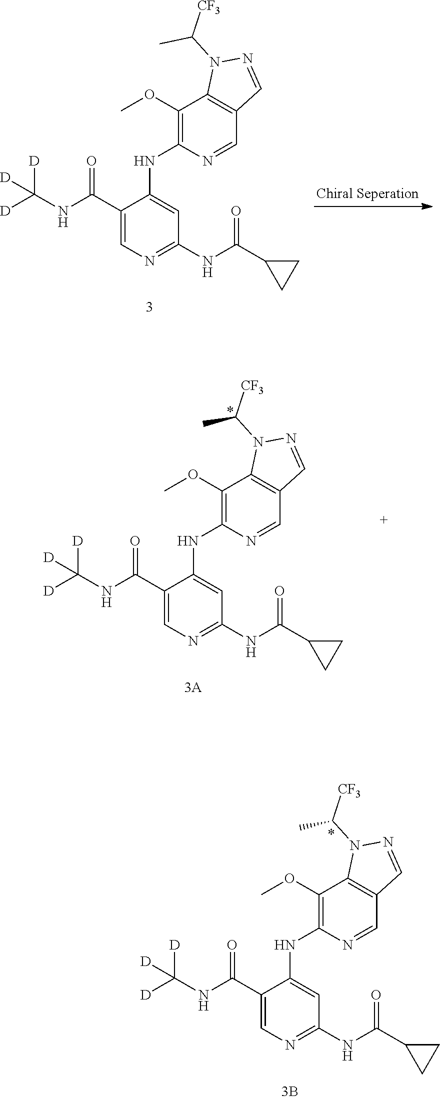 [SMILES: [2H]C([2H])([2H])NC(=O)c1cnc(NC(=O)C2CC2)cc1Nc1ncc2cnn(C(C)C(F)(F)F)c2c1OC ; [2H]C([2H])([2H])NC(=O)c1cnc(NC(=O)C2CC2)cc1Nc1ncc2cnn([C@@H](C)C(F)(F)F)c2c1OC ; [2H]C([2H])([2H])NC(=O)c1cnc(NC(=O)C2CC2)cc1Nc1ncc2cnn([C@H](C)C(F)(F)F)c2c1OC]

### Step 1. 6-Chloro-7-methoxy-1-(1,1,1-trifluoropropan-2-yl)-1H-pyrazolo[4,3-c]pyridine (3a)

To a solution of 1d (1 g, 5.45 mmol), Cs₂CO₃ (5.32 g, 16.34 mmol) in DMF (10 mL) was added Int. B (27 mL, 10.87 mmol, 0.4 M in DCM). Then mixture was stirred at r.t. for 2 h. The reaction mixture was diluted with H₂O (50 mL) and extracted with DCM (20 mL*3). The organic layer was washed with brine (20 mL), dried over Na₂SO₄ and filtered. The filtrate was concentrated and the residue was purified by flash chromatography on silical gel (PE/EA=3/1) to afford compound 3a (160 mg, 10% yield) as a yellow solid. ¹H NMR (400 MHz, CDCl₃) δ 8.64 (s, 1H), 8.20 (s, 1H), 5.77-5.66 (m, 1H), 4.09 (s, 3H), 1.89 (d, J=7.2 Hz, 3H). LC-MS (ESI, Method 4) t_{R}=2.91 min, m/z (M+H)⁺=280.1.

### Step 2. 7-Methoxy-N-(4-methoxybenzyl)-1-(1,1,1-trifluoropropan-2-yl)-1H-pyrazolo[4,3-c]pyridin-6-amine (3b)

A mixture of 3a (160 mg, 0.57 mmol), (4-methoxyphenyl)methanamine (157 mg, 1.14 mmol), ^{t}BuONa (110 mg, 1.14 mmol), BrettPhos (61 mg, 0.11 mmol), BrettPhos Pd G3 (52 mg, 0.057 mmol) in dioxane (2 mL) was stirred at 120° C. for 24 h. The mixture was diluted with H₂O (10 mL), extracted with EtOAc (10 mL*3), washed with brine (20 mL), dried over Na₂SO₄ and filtered. The filtrate was concentrated and purified by flash chromatography (DCM/MeOH=100/1 to 30/1) to get compound 3b (140 mg, 64% yield) as a yellow oil. LC-MS (ESI, Method 4) t_{R}=3.11 min, m/z (M+H)⁺=381.2.

### Step 3. 7-Methoxy-1-(1,1,1-trifluoropropan-2-yl)-1H-pyrazolo[4,3-c]pyridin-6-amine (3c)

A solution of 3b (140 mg, 0.37 mmol) in TFA (2 mL) was stirred at r.t. for 16 h. The mixture was concentrated and diluted with H₂O (10 mL), basified to pH >7 with aq. Na₂CO₃, extracted with EtOAc (10 mL*3), washed with brine (15 mL), dried over Na₂SO₄, concentrated and purified by flash chromatography (DCM/MeOH=50/1 to 5/1) to get compound 3c (70 mg, 73% yield) as a yellow solid. LC-MS (ESI, Method 4) t_{R}=0.81 min, m/z (M+H)⁺=261.1.

### Step 4. 6-Chloro-4-((7-methoxy-1-(1,1,1-trifluoropropan-2-yl)-1H-pyrazolo[4,3-c]pyridin-6-yl)amino)-N-(methyl-d₃)nicotinamide (3d)

To a solution of 4,6-dichloro-N-(methyl-d₃)nicotinamide (29 mg, 0.14 mmol) and 3c (30 mg, 0.115 mmol) in DMF (2 mL) was added NaH (23 mg, 0.58 mmol, 60% purity in mineral oil) at 0° C. Then the mixture was stirred at 0° C. for 2 h and stirred at r.t. for 16 h. The mixture was diluted with H₂O (10 mL), extracted with EtOAc (10 mL*3), washed with brine (20 mL), dried over Na₂SO₄ and filtered. The filtrate was concentrated and purified by flash chromatography (MeOH in DCM is 0-6%) to get compound 3d (25 mg, 50% yield) as a yellow solid. LC-MS (ESI, Method 4) t_{R}=3.15 min, m/z (M+H)⁺=432.2.

### Step 5. 6-(Cyclopropanecarboxamido)-4-((7-methoxy-1-(1,1,1-trifluoropropan-2-yl)-1H-pyrazolo[4,3-c]pyridin-6-yl)amino)-N-(methyl-d₃)nicotinamide (3)

A mixture of 3d (25 mg, 0.058 mmol), cyclopropanecarboxamide (99 mg, 1.16 mmol), BrettPhos Pd G3 (5 mg, 0.006 mmol), BrettPhos (3 mg, 0.006 mmol), Cs₂CO₃ (47 mg, 0.14 mmol) in dioxane (0.5 mL) was stirred at 120° C. for 1 h under M.W. The mixture was diluted with H₂O (10 mL), extracted with EtOAc (10 mL*3), washed with brine (20 mL), dried over Na₂SO₄ and filtered. The filtrate was concentrated and purified by prep-HPLC (Method E) to get compound 3 (8 mg, 29% yield) as a pale-yellow solid. ¹H NMR (400 MHz, DMSO-d₆) δ 11.71 (s, 1H), 10.74 (s, 1H), 9.25 (s, 1H), 8.71 (s, 1H), 8.69 (s, 1H), 8.57 (s, 1H), 8.40 (s, 1H), 5.69-5.65 (m, 1H), 3.91 (s, 3H), 2.04-2.00 (m, 1H), 1.85 (d, J=6.8 Hz, 3H), 0.81-0.78 (m, 4H). LC-MS (ESI, Method 4) t_{R}=2.26 min, m/z (M+H)⁺=481.3.

### Step 6. (R*)-6(Cyclopropanecarboxamido)-4((7-methoxy-1-(1,1,1-trifuoropropan-2-yl)-1H-pyrazolo[4,3-c]pyridin-6-yl)amino)-N-(methyl-d₃)nicotinamide (3A) and (S*)-6-(Cyclopropanecarboxamido)-4-((7-methoxy-1-(1,1,1-trifluoropropan-2-yl)-1H-pyrazolo[4,3-c]pyridin-6-yl)amino)-N-(methyl-d₃)nicotinamide (3B)

3 (8 mg, 0.017 mmol) was separated by chiral prep-HPLC (Method G) to afford 3A (2.5 mg, 31% yield) as a white solid and 3B (2.4 mg, 30% yield) as a white solid. 3A: LC-MS (ESI, Method 4) t_{R}=2.25 min, m/z (M+H)⁺=481.2. HPLC (Method 5) t_{R}=5.92 min. 3B: LC-MS (ESI, Method 4) t_{R}=2.26 min, m/z (M+H)⁺=481.3. HPLC (Method 5) t_{R}=7.89 min.

### Example 4

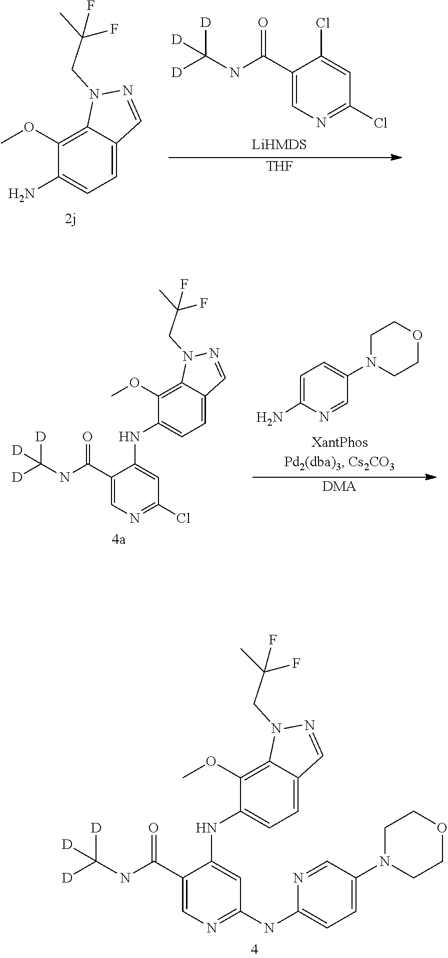

### Step 1. 6-Chloro-4-((1-(2,2-difluoropropyl)-7-methoxy-1H-indazol-6-yl)amino)-N-(methyl-d3)nicotinamide (4a)

To a solution of 2j (30 mg, 0.12 mmol) and 4,6-dichloro-N-(methyl-d₃)nicotinamide (25.9 mg, 0.12 mmol) in THF (1 mL) was added LiHMDS (0.87 mL, 0.87 mmol, 1 M in THF) at 0° C. Then the mixture was stirred at r.t. for 2 h. The mixture was diluted with H₂O (10 mL), extracted with EtOAc (10 mL*3), washed with brine (20 mL), dried over Na₂SO₄ and filtered. The filtrate was concentrated and purified by flash chromatography (DCM/MeOH=100/1 to 30/1) to get compound 4a (50 mg, 97% yield) as a yellow solid. LC-MS (ESI, Method 4) t_{R}=2.74 min, m/z (M+H)⁺=413.2.

### Step 2. 4-((1-(2,2-Difluoropropyl)-7-methoxy-1H-indazol-6-yl)amino)-N-(methyl-d₃)-6-((5-morpholinopyridin-2-yl)amino)nicotinamide (4)

Compound 4a (50 mg, 0.12 mmol), 5-morpholinopyridin-2-amine (65 mg, 0.36 mmol), XantPhos (14 mg, 0.024 mmol), Cs₂CO₃ (99 mg, 0.30 mmol) and Pd₂(dba)₃ (11 mg, 0.012 mmol) were dissolved in DMA (1 mL). The resulting mixture was stirred at 145° C. for 2 h. The mixture was concentrated to dryness and purified by prep-HPLC (Method D) to give the title compound 4 (11.4 mg, 17% yield) as a pale-yellow solid. ¹H NMR (400 MHz, DMSO-d₆) δ 10.59 (s, 1H), 9.43 (s, 1H), 8.47 (s, 1H), 8.45 (s, 1H), 8.16 (s, 1H), 7.77 (d, J=3.2 Hz, 1H), 7.59 (d, J=8.8 Hz, 1H), 7.52-7.50 (m, 2H), 7.37 (dd, J=9.2 Hz, J=2.8 Hz, 1H), 7.32 (d, J=8.8 Hz, 1H), 4.98 (t, J=13.2 Hz, 2H), 3.81 (s, 3H), 3.73-3.71 (m, 4H), 3.03-3.01 (m, 4H), 1.67 (t, J=19.2 Hz, 3H). LC-MS (ESI, Method 4) t_{R}=2.27 min, m/z (M+H)⁺=556.4.

### Example 5

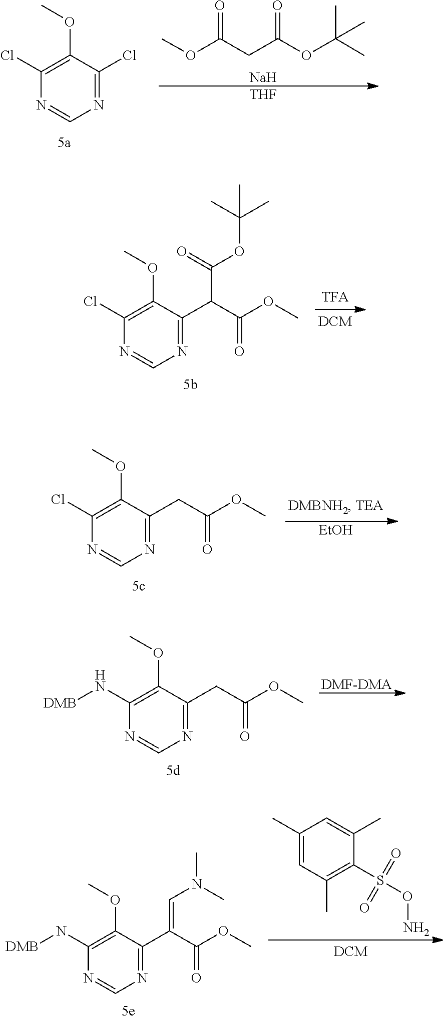

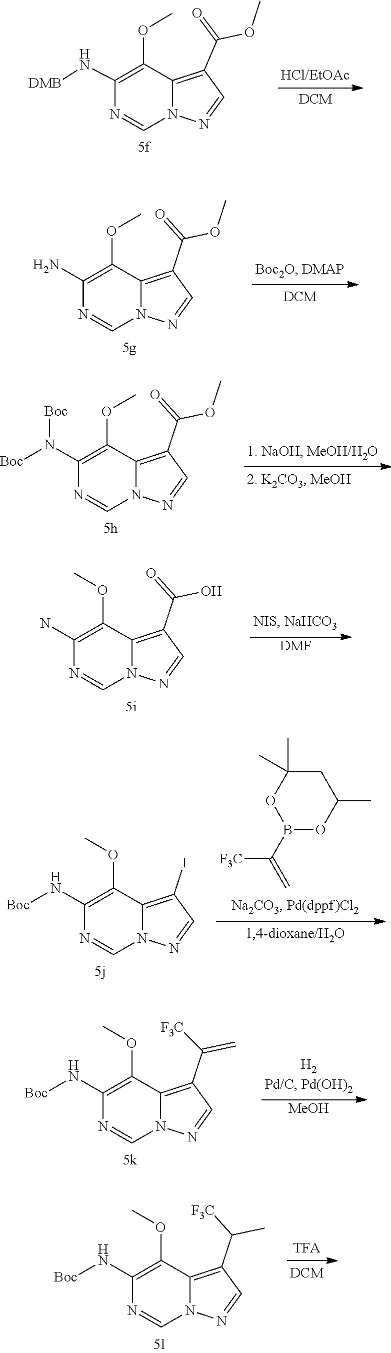

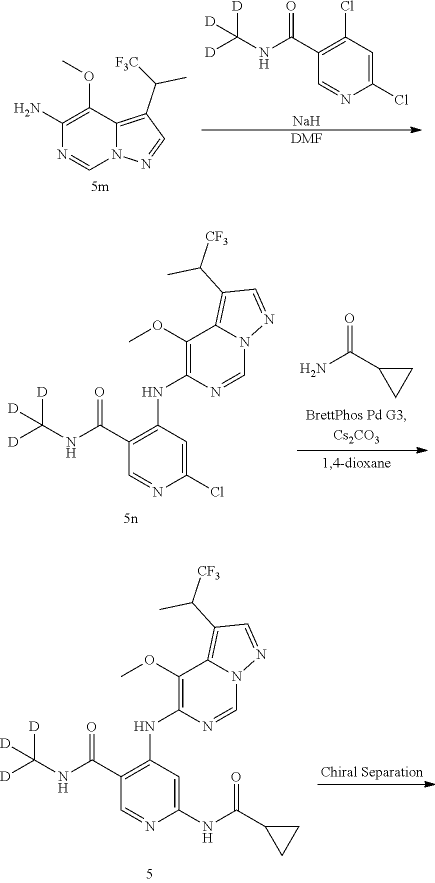

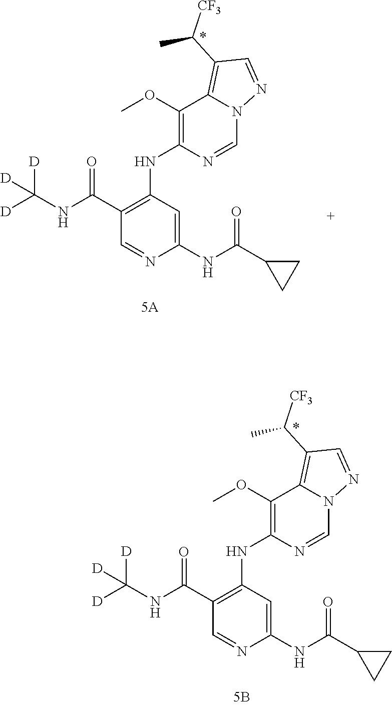 [SMILES: [2H]C([2H])([2H])NC(=O)c1cnc(NC(=O)C2CC2)cc1Nc1ncn2ncc([C@@H](C)C(F)(F)F)c2c1OC ; [2H]C([2H])([2H])NC(=O)c1cnc(NC(=O)C2CC2)cc1Nc1ncn2ncc([C@H](C)C(F)(F)F)c2c1OC]

### Step 1. 1-Tert-butyl 3-methyl 2-(6-chloro-5-methoxypyrimidin-4-yl) malonate (5b)

To a solution of tert-butyl methyl malonate (53.52 g, 307 mmol) in THF (600 mL) was added NaH (22.35 g, 559 mmol, 60% purity in mineral oil) at 0° C. The mixture was stirred at 0° C. for 30 min. Then a solution of 5a (50 g, 279 mmol) in THF (100 mL) was added to the mixture at 0° C. The mixture was stirred at 80° C. for 2 h and poured into ice-water (400 mL). The mixture was acidified with 2 N HCl to adjust to pH=2 and extracted with EtOAc (500 mL*2). The organic layer was washed with brine (300 mL), dried over Na₂SO₄, filtered and concentrated to afford 5b (88 g, 100% yield) as a yellow oil, which was used for the next step directly without further purification. LC-MS (ESI, Method 3) t_{R}=1.33 min, m/z (M+H)+=317.2.

### Step 2. Methyl 2-(6-chloro-5-methoxypyrimidin-4-yl)acetate (5c)

A mixture of 5b (70 g, 177 mmol) in HCl/EtOAc (2 M, 400 mL) was stirred at 30° C. overnight. The reaction mixture was concentrated. The residue was diluted with EtOAc (100 mL), adjusted to pH=10 with sat. Na₂CO₃ (150 mL). The mixture was extracted with EtOAc (200 mL*3). The combined organic layer was dried over Na₂SO₄, filtered and concentrated to afford 5c (38 g, 100% yield) as a yellow oil, which was used for the next step directly without further purification. LC-MS (ESI, Method 3) t_{R}=1.05 min, m/z (M+H)⁺=217.2.

### Step 3. Methyl 2-(6-((2,4-dimethoxybenzyl)amino)-5-methoxypyrimidin-4-yl)acetate (5d)

A mixture of 5c (54 g, 199 mmol), TEA (40 g, 399 mmol) and DMBNH₂(37 g, 219 mol) in EtOH (300 mL) was stirred at 80° C. for 5 h. After cooling to 5° C., the formed solid was collected by filtering and dried to afford 5d (1.33 g, 83% yield) as a white solid, which was used for the next step directly without further purification. LC-MS (ESI, Method 3) t_{R}=1.05 min, m/z (M+H)⁺=348.2.

### Step 4. Methyl 2-(6-((2,4-dimethoxybenzyl)amino)-5-methoxypyrimidin-4-yl)-3-(dimethylamino)acrylate (5e)

A mixture of 5d (17.5 g, 50.38 mmol) and DMF-DMA (18.05 g, 151 mmol) was stirred at 130° C. for 6 h. The mixture was concentrated to afford crude 5e (20.27 g, yield given) as a brown oil, which was used for the next step directly without further purification. LC-MS (ESI, Method 3) t_{R}=1.38 min, m/z (M+H)⁺=403.3.

### Step 5. Methyl 5-((2,4-dimethoxybenzyl)amino)-4-methoxypyrazolo[1,5-c]pyrimidine-3-carboxylate (5f)

To a mixture of 5e (21.5 g crude, 48.1 mmol) in DCM (200 mL) was added dropwise a solution of O-(mesitylsulfonyl)hydroxylamine (28.75 g, 80.14 mmol, 60% wt in H₂O) in DCM (100 mL) at 0° C. After stirring at 0° C. for 2 h, the reaction mixture was poured into ice-sat.NaHCO₃ (70 mL) and extracted with DCM (150 mL*2). The combined organic layer was concentrated and the residue was purified by flash chromatography on silica gel (DCM/EtOAc=5/1) to afford 5f (11.25 g, 63% yield) as a yellow solid. ¹H NMR (400 MHz, CDCl₃) δ 8.85 (s, 1H), 8.27 (s, 1H), 7.21 (d, J=8.4 Hz, 1H), 6.46 (d, J=2.4 Hz, 1H), 6.41 (dd, J=8.4, 2.4 Hz, 1H), 5.64 (t, J=6.0 Hz, 1H), 4.64 (d, J=6.0 Hz, 2H), 3.86 (s, 3H), 3.85 (s, 3H), 3.80 (s, 3H), 3.79 (s, 3H).

### Step 6. Methyl 5-amino-4-methoxypyrazolo[1,5-c]pyrimidine-3-carboxylate (5g)

To a solution of 5f (17.7 g, 47.53 mmol) in DCM (40 mL) was added HCl/EtOAc (120 mL, 2 M). The mixture was stirred at 30° C. for 1 h. The mixture was concentrated and the residue was diluted with H₂O (100 mL). The suspension was basidied with 2 M aq. NaOH to pH=13. The solid was filtered and the filter cake was treated with DCM/MeOH (200 mL) at refluxing for 1 h. The solid was filtered off and the filtrate was concentrated to afford 5g (8.2 g, 78% yield) as a yellow solid. ¹H NMR (400 MHz, CDCl₃) δ 8.81 (s, 1H), 8.32 (s, 1H), 4.91 (s, 2H), 3.88 (s, 6H).

### Step 7. Methyl 5-[bis(tert-butoxycarbonyl)amino]-4-methoxy-pyrazolo[1,5-c]pyrimidine-3-carboxylate (5h)

A mixture of 5g (8.2 g, 36.94 mmol), Boc₂O (20.15 g, 92.34 mmol) and DMAP (901 mg, 7.29 mmol) in DCM (125 mL) was stirred at 30° C. for 6 h. The solvent was removed in vacuo and the residue was purified by flash chromatography on silica gel (DCM/MeOH=20/1) to afford 5h (14.4 g, 92% yield) as a yellow oil. ¹H NMR (400 MHz, CDCl₃) δ 9.05 (s, 1H), 8.50 (s, 1H), 3.95 (s, 3H), 3.92 (s, 3H), 1.46 (s, 18H).

### Step 8. 5-((Tert-butoxycarbonyl)amino)-4-methoxypyrazolo[1,5-c]pyrimidine-3-carboxylic acid (5i)

To a mixture of 5h (13.5 g, 31.96 mmol) in MeOH (200 mL) and H₂O (100 mL) was added NaOH (1.92 g, 47.94 mmol) at r.t. The mixture was stirred at 40° C. for 10 h. After cooling to r.t., the organic layer was removed under reduced pressure and the aqueous layer was acidified by 2 N aq. HCl to pH=1. The formed solid was filtered and dried to afford a white solid. The obtained white solid, K₂CO₃(12.17 g, 88.03 mmol) and MeOH (80 mL) was stirred at 70° C. for 3 h. Then, the mixture was concentrated and the residue was acidified by 2 N aq. HCl to pH=1. The formed solid was filtered and dried to afford 5i (7.42 g, 71% yield) as a white solid. ¹H NMR (400 MHz, CDCl₃) δ 9.12 (s, 1H), 8.52 (s, 1H), 7.33 (s, 1H), 3.97 (s, 3H), 1.57 (s, 9H).

### Step 9. Tert-butyl (3-iodo-4-methoxypyrazolo[1,5-c]pyrimidin-5-yl)carbamate (5j)

To a mixture of 5i (3.3 g, 10.70 mmol) and NIS (4.82 g, 21.41 mmol) in DMF (45 mL) was added NaHCO₃ (2.70 g, 32.11 mmol) at 0° C. After stirring at 10° C. for 2 h, the reaction mixture was diluted with aq. Na₂S₂O₃ (350 mL) and extracted with EtOAc (100 mL*3). The organic layer was washed with brine (100 mL*3), dried over Na₂SO₄, filtered and the filtrate was concentrated to afford 5j (2.97 g, 71% yield) as a yellow solid. ¹H NMR (400 MHz, DMSO-d₆) δ 9.28 (s, 1H), 9.19 (s, 1H), 8.21 (s, 1H), 3.83 (s, 3H), 1.49 (s, 9H).

### Step 10. Tert-butyl (4-methoxy-3-(3,3,3-trifluoroprop-1-en-2-yl)pyrazolo[1,5-c]pyrimidin-5-yl)carbamate (5k)

A mixture of 5j (145 mg, 0.37 mmol), 4,4,6-trimethyl-2-[1-(trifluoromethyl)vinyl]-1,3,2-dioxaborinane (165 mg, 0.74 mmol), Na₂CO₃ (79 mg, 0.74 mmol) and Pd(dppf)Cl₂ (27 mg, 0.04 mmol) in 1,4-dioxane/H₂O (0.5 mL/0.1 mL) was stirred at 70° C. for 6 h and 90° C. for 3 h. After cooling to r.t., the reaction mixture was diluted with EtOAc (10 mL), washed with water (5 mL) and brine (5 mL). The organic layer dried over Na₂SO₄, filtered and the filtrate was concentrated to afford 5k (130 mg, crude) as a yellow oil, which was used for the next step directly without further purification. LC-MS (ESI, Method 3) t_{R}=1.28 min, m/z (M−Bu+H)⁺=303.1.

### Step 11. Tert-butyl (4-methoxy-3-(1,1,1-trifluoropropan-2-yl)pyrazolo[1,5-c]pyrimidin-5-yl)carbamate (51)

A mixture of 5k (130 mg, 0.36 mmol), Pd/C (150 mg, 50% wt. wetted in water) and Pd(OH)₂ (150 mg, Pd 20% on carbon, wetted in water 50% water) in MeOH (2 mL) was stirred at 50° C. for 3 days under H₂ (50 Psi) atmosphere. The mixture was filtered and the filtrate was concentrated to afford 51(130 mg, crude) as a yellow oil, which was used for the next step directly without further purification. LC-MS (ESI, Method 3) t_{R}=1.35 min, m/z (M-Bu+H)⁺=305.0.

### Step 12. 4-Methoxy-3-(1,1,1-trifluoropropan-2-yl)pyrazolo[1,5-c]pyrimidin-5-amine (5m)

A mixture of 5l (125 mg, 0.35 mmol) and TFA/DCM (2 mL/6 mL) was stirred at 0° C. for 1 h. The solvent was removed by pumping nitrogen stream. The residue was diluted with DCM (3 mL), adjusted to pH >7 with NH₃/1,4-dioxane (2 M). The mixture was concentrated and the residue was purified by flash chromatography on silical gel (DCM/EtOAc=10/1) to afford 5m (59 mg, 65% yield) as a yellow oil. LC-MS (ESI, Method 3) t_{R}=1.17 min, m/z (M+H)⁺=261.0.

### Step 13. 6-Chloro-4-((4-methoxy-3-(1,1,1-trifluoropropan-2-yl)pyrazolo[1,5-c]pyrimidin-5-yl)amino)-N-(methyl-d₃)nicotinamide (5n)

To a solution of 5m (50 mg, 0.19 mmol) and 4,6-dichloro-N-(methyl-d₃)nicotinamide (120 mg, 0.58 mmol) in DMF (1 mL) was added NaH (23 mg, 0.58 mmol, 60% in mineral oil) at 0° C. Then the mixture was stirred at 15° C. for 3 h. The reaction mixture was quenched with ice-water (3 mL) and extracted with EtOAc (5 mL*3). The organic layer was concentrated and the residue was purified by prep-HPLC (Method A) to afford 5n (10 mg, 12% yield) as a white solid. LC-MS (ESI, Method 3) t_{R}=1.26 min, m/z (M+H)⁺=432.3.

### Step 14. 6-(Cyclopropanecarboxamido)-4-((4-methoxy-3-(1,1,1-trifluoropropan-2-yl)pyrazolo[1,5-c]pyrimidin-5-yl)amino)-N-(methyl-d₃)nicotinamide (5)

A mixture of Sn (10.0 mg, 0.023 mmol), cyclopropanecarboxamide (10.0 mg, 0.12 mmol), Cs₂CO₃ (15 mg, 0.046 mmol) and Brettphos Pd G3 (4 mg, 0.005 mmol) in 1,4-dioxane (0.5 mL) was stirred at 90° C. for 10 h under N₂ atmosphere. The reaction mixture was concentrated and the residue was purified by prep-HPLC (Method A) to afford 5 (3 mg, 27% yield) as a white solid. ¹H NMR (400 MHz, DMSO-d₆) δ 11.54 (s, 1H), 10.76 (s, 1H), 9.33 (s, 1H), 8.86 (s, 1H), 8.69 (s, 1H), 8.58 (s, 1H), 8.26 (s, 1H), 4.11-4.05 (m, 1H), 3.87 (s, 3H), 2.02-1.96 (m, 1H), 1.55 (d, J=7.2 Hz, 3H), 0.81-0.79 (m, 4H). LC-MS (ESI, Method 2) t_{R}=3.25 min, m/z (M+H)⁺=481.1.

### Step 15. (R*)-6-(Cyclopropanecarboxamido)-4((4-methoxy-3-(1,1,1-trifluoropropan-2-yl)pyrazolo[1,5-c]pyrimidin-5-yl)amino)-N-(methyl-d₃)nicotinamide (5A) and (S*)-6-(Cyclopropanecarboxamido)-4-((4-methoxy-3-(1,1,1-trifluoropropan-2-yl)pyrazolo[1,5-c]pyrimidin-5-yl)amino)-N-(methyl-d₃)nicotinamide (5B)

Compound 5 (10 mg, 0.021 mmol) was separated by chiral prep-HPLC (Method H) to afford 5A (3 mg, 27% yield) as a white solid and 5B (3 mg, 27% yield) as a white solid.

5A: ¹H NMR (400 MHz, DMSO-d₆) δ 11.54 (s, 1H), 10.76 (s, 1H), 9.33 (s, 1H), 8.85 (s, 1H), 8.70 (s, 1H), 8.58 (s, 1H), 8.26 (s, 1H), 4.11-4.07 (m, 1H), 3.87 (s, 3H), 2.02-1.97 (m, 1H), 1.55 (d, J=7.2 Hz, 3H), 0.81-0.79 (m, 4H). LC-MS (ESI, Method 2) t_{R}=3.23 min, m/z (M+H)⁺=481.0. HPLC (Method 6) t_{R}=5.00 min.

5B: ¹H NMR (400 MHz, DMSO-d₆) δ 11.54 (s, 1H), 10.76 (s, 1H), 9.33 (s, 1H), 8.85 (s, 1H), 8.69 (s, 1H), 8.58 (s, 1H), 8.26 (s, 1H), 4.11-4.07 (m, 1H), 3.87 (s, 3H), 2.02-1.97 (m, 1H), 1.55 (d, J=7.2 Hz, 3H), 0.81-0.79 (m, 4H). LC-MS (ESI, Method 2) t_{R}=3.24 min, m/z (M+H)⁺=481.0. HPLC (Method 6) t_{R}=7.03 min.

### Example 6

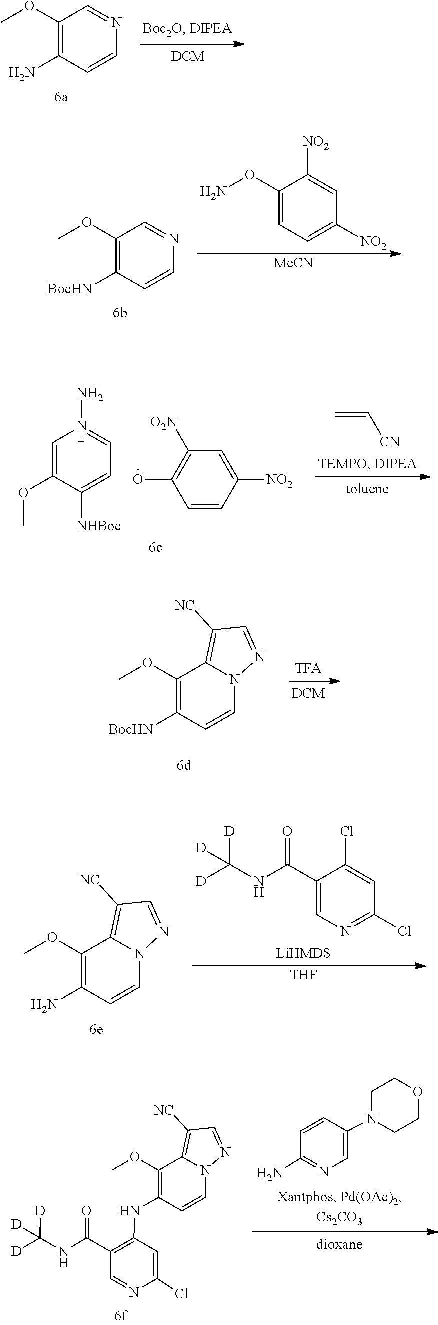

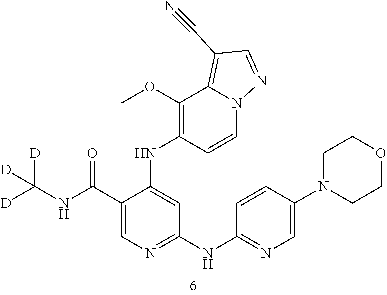 [SMILES: [2H]C([2H])([2H])NC(=O)c1cnc(Nc2ccc(N3CCOCC3)cn2)cc1Nc1ccn2ncc(C#N)c2c1OC]

### Step 1. Tert-butyl (3-methoxypyridin-4-yl)carbamate (6b)

A solution of 6a (800 mg, 6.44 mmol), Boc₂O (1.83 g, 8.38 mmol, 1.9 mL) and DIPEA (1.67g 12.89 mmol, 2.24 mL) in DCM (15 mL) was stirred at 20° C. for 12 h. A yellow solution was formed. The reaction mixture was concentrated and purified by flash chromatography (EtOAc in PE is 10-50%) to give 6b (1.45 g, yield given) as a white solid. LC-MS (ESI, Method 4) t_{R}=1.92 mmi, m/z (M+H)⁺=225.1.

### Step 2. 1-Amino-4-((tert-butoxycarbonyl)amino)-3-methoxypyridin-1-ium 2,4-dinitrophenolate (6c)

A mixture of 6b (1.45 g, 6.47 mmol) and O-(2,4-dinitrophenyl) hydroxylamine (1.42 g, 7.11 mmol) in MeCN (50 mL) was stirred at 504C for 16 h. A yellow solution was formed. The reaction was concentrated to give 6c (2.73 g, crude) as a yellow solid, which was used for the next step directly without further purification. LC-MS (ESI, Method 4) t_{R}=1.66 mg c m/z M=240.1.

### Step 3. Tert-butyl (3-cyano-4-methoxypyrazolo[1,5-a]pyridin-5-yl) carbamate (6d)

A mixture of 6c (88 mg, 0.208 mmol), acrylonitrile (27 mg, 0.312 mmol), TEMPO (40 mg, 0.25 mmol) and DIPEA (54 mg, 0.416 mmol, 73 μL) in toluene (1 mL) was stirred at 40° C. for 12 h. A black solution was formed. The reaction mixture was diluted with water (30 mL) and extracted with EtOAc (30 mL*2). The combined organic layer was washed with brine (40 mL*2), dried over anhydrous Na₂SO₄, filtered and concentrated. The residue was purified by column chromatograph (EtOAc in PE is 10-50%) to give 6d (30 mg, 50% yield over 2 steps) as a yellow solid. ¹H NMR (400 MHz, CDCl₃) δ 8.30 (d, J=7.2 Hz, 1H), 8.14 (s, 1H), 8.08 (d, J=7.6 Hz, 1H), 7.22 (brs, 1H), 4.04 (s, 3H), 1.56 (s, 9H). LC-MS (ESI, Method 4) t_{R}=4.02 min, m/z (M+H)⁺=289.1.

### Step 4. 5-Amino-4-methoxypyrazolo[1,5-a]pyridine-3-carbonitrile (6e)

To a mixture of 6d (30 mg, 0.104 mmol) in DCM (3 mL) was added TFA (1.49 g, 13.1 mmol, 1 mL) at 0° C. The resulting mixture was stirred at 20° C. for 1 h. The reaction mixture was concentrated and diluted with H₂O (10 mL), adjusted pH >7 with aq Na₂CO₃, extracted with EtOAc (10 mL*3) washed with brine, dried in vacuo to give 6e (18 mg, 92% yield) as a yellow solid. LC-MS (ESI, Method 4) t_{R}=1.97 min, m/z (M+H)⁺=189.0.

### Step 5. 6-Chloro-4-((3-cyano-4-methoxypyrazolo[1,5-a]pyridin-5-yl)amino)-N-(methyl-d3)nicotinamide (6f)

To a solution of 6e (18 mg, 0.096 mmol) and 4,6-dichloro-N-(methyl-d₃)nicotinamide (20 mg, 0.096 mmol) in THF (0.5 mL) was added LiHMDS (0.67 mL, 0.67 mmol, 1 M in THF) at 0° C. The resulting mixture was stirred at 10° C. for 2 h. The reaction mixture was diluted with H₂O (10 mL), extracted with EtOAc (10 mL*3) washed with brine (10 mL), dried in vacuo to give 6f (30 mg, 88% yield) as a yellow solid. LC-MS (ESI, Method 4) t_{R}=2.41 min, m/z (M+H)⁺=360.1.

### Step 6. 4-((3-Cyano-4-methoxypyrazolo[1,5-a]pyridin-5-yl)amino)-N-(methyl-d3)-6-((5-morpholinopyridin-2-yl)amino)nicotinamide (6)

To a solution 6f (30 mg, 0.083 mmol) and 5-morpholinopyridin-2-amine (30 mg, 0.17 mmol) in dioxane (0.5 mL) was added XantPhos (10 mg, 0.017 mmol) and Cs₂CO₃ (54 mg, 0.17 mmol) and Pd(OAc)₂(2 mg, 0.008 mmol) at 25° C., the reaction mixture was stirred at 100° C. for 12 hours under N₂ atmosphere, the mixture was diluted with H₂O (2 mL), extracted with EtOAc (10 mL*3), washed with brine (10 mL), dried over Na₂SO₄, concentrated and purified by prep-HPLC (Method E) to get the compound 6 (6.5 mg, 16% yield) as a yellow solid. ¹H NMR (400 MHz, DMSO) δ 11.10 (s, 1H), 9.71 (s, 1H), 8.55 (s, 1H), 8.49 (s, 1H), 8.14 (s, 1H), 7.95-7.93 (m, 2H), 7.75 (d, J=6.4 Hz, 1H), 7.48 (d, J=9.2 Hz, 1H), 7.41 (dd, J=9.2 Hz, 2.8 Hz, 1H), 6.95 (d, J=6.4 Hz, 1H), 3.74 (t, J=4.8 Hz, 4H), 3.66 (s, 3H), 3.03 (t, J=4.8 Hz, 4H). LC-MS (ESI, Method 4) t_{R}=1.72 min, m/z (M+H)⁺=503.3.

### Example 7

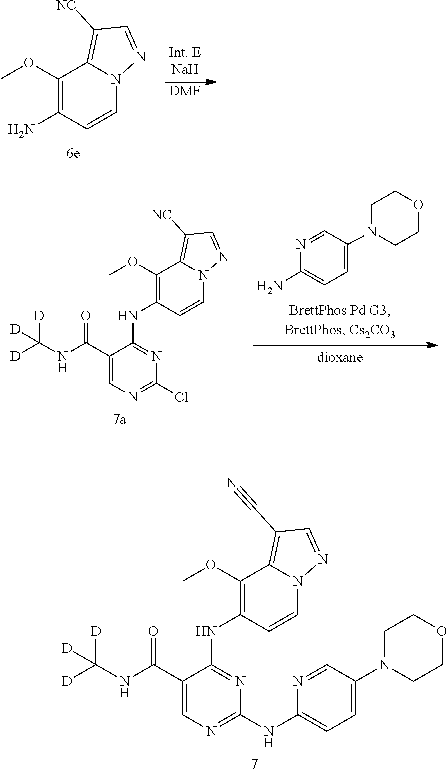

### Step 1. 2-Chloro-4-((3-cyano-4-methoxypyrazolo[1,5-a]pyridin-5-yl)amino)-N-(methyl-d3)pyrimidine-5-carboxamide (7a)

To a mixture of Int. E (45 mg, 0.212 mmol) and 6e (40 mg, 0.212 mmol) in DMF (3 mL) was added NaH (60 mg, 1.49 mmol, 60% in mineral oil) at 0° C. The resulting mixture was stirred at 0-20° C. for 2 h. The reaction mixture was quenched with water (30 mL) and extracted with EtOAc (30 mL*3). The combined organic layer was washed with water (30 mL) and brine (30 mL), dried over anhydrous Na₂SO₄ and concentrated to give the crude product. The crude product was purified by prep-TLC (DCM/MeOH=10/1) to give 7a (30 mg, 39% yield) as a yellow solid. LC-MS (ESI, Method 4), t_{R}=2.53 min, m/z (M+H)⁺=361.2.

### Step 2 4-((3-Cyano-4-methoxypyrazolo[1,5-a]pyridin-5-yl)amino)-N-(methyl-d)-2-((5-morpholinopyridin-2-yl)amino)pyrimidine-5-carboxamide (7)

A mixture of 7a (30 mg, 0.083 mmol), 5-morpholinopyridin-2-amine (18 mg, 0.10 mmol), BrettPhos (10 mg, 0.017 mmol), Cs₂CO₃ (81 mg, 0.25 mmol) and BrettPhos Pd G3 (8 mg, 0.008 mmol) in dioxane (1 mL) was degassed and purged with nitrogen for 3 times. The resulting mixture was stirred at 100° C. under N₂ atmosphere for 1 h. A yellow suspension was formed. The reaction mixture was concentrated and purified by prep-HPLC (Method E) to give compound 7 (1.6 mg, 3.8% yield) as an off-white solid. ¹H NMR (400 MHz, DMSO-d₆) δ 12.29 (s, 1H), 10.14 (s, 1H), 9.03 (d, J=6.8 Hz, 1H), 8.76 (s, 1H), 8.67 (d, J=7.6 Hz, 1H), 8.62 (s, 1H), 8.56 (s, 1H), 8.10 (d, J=2.8 Hz, 1H), 7.84 (d, J=6.8 Hz, 1H), 7.46 (dd, J=9.2, 3.2 Hz, 1H), 3.98 (s, 3H), 3.77 (t, J=4.8 Hz, 4H), 3.15 (t, J=4.8 Hz, 4H). LC-MS (ESI, Method 4) t_{R}=2.43 min, m/z (M+H)⁺=504.3.

### Example 8

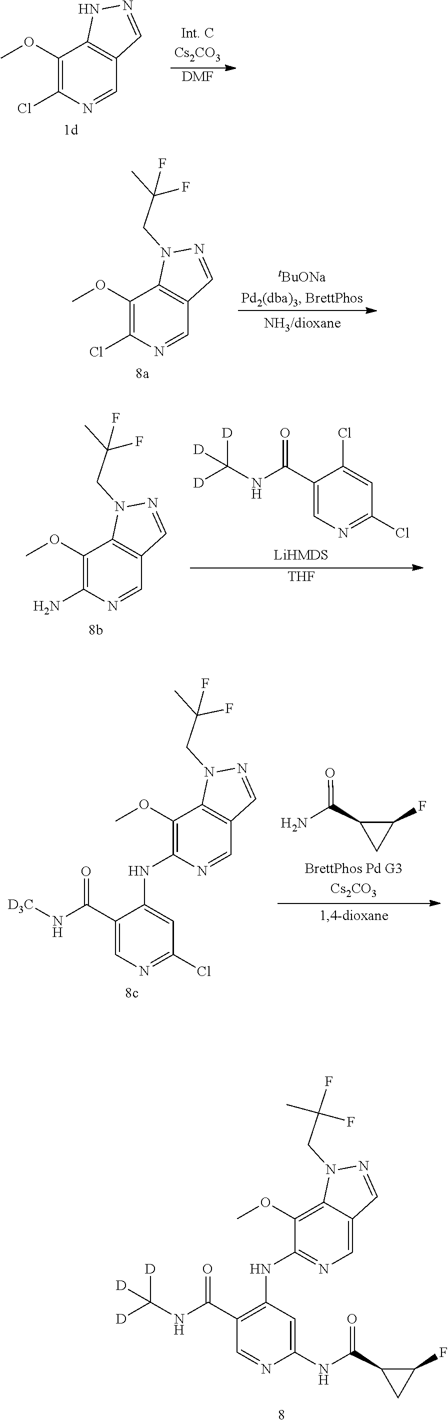

### Step 1. 6-Chloro-1-(2,2-difluoropropyl)-7-methoxy-1H-pyrazolo[4,3-c]pyridine (8a)

A mixture of 1d (400 mg, 2.18 mmol), Int. C (1.6 g, 7.01 mmol), Cs₂CO₃ (1.42 g, 4.36 mmol) in DMF (5 mL) was stirred at 30° C. for 1 h. The reaction mixture was diluted with H₂O (15 mL) and extracted with EtOAc (15 mL*3). The combined organic layer was washed with brine (20 mL), dried over Na₂SO₄ and filtered. The filtrate was concentrated and the residue was purified by flash chromatography on silical gel (PE/EA=3/1) to afford 8a (120 mg, 21% yield) as a yellow oil. ¹H NMR (400 MHz, DMSO-d₆) δ 8.73 (s, 1H), 8.45 (s, 1H), 4.98 (t, J=13.6 Hz, 2H), 4.02 (s, 3H), 1.71 (t, J=19.2 Hz, 3H).

### Step 2. 1-(2,2-Difluoropropyl)-7-methoxy-1H-pyrazolo[4,3-c]pyridin-6-amine (8b)

A mixture of 8a (266 mg, 1 mmol), Pd₂(dba)₃ (46 mg, 0.05 mmol), BrettPhos (27 mg, 0.050 mmol) and BuONa (147 mg, 1.52 mmol) in sat. NH₃/dioxane (4 mL) was stirred at 100° C. for 12 h under N₂. The mixture was cooled and concentrated. The residue was purified by prep-HPLC (Method A) to afford 8b (100 mg, 41% yield) as a white solid. LC-MS (ESI, Method 3) t_{R}=1.27 min, m/z (M+H)⁺=243.2.

### Step 3. 6-Chloro-4-((1-(2,2-difluoropropyl)-7-methoxy-1H-pyrazolo[4,3-c]pyridin-6-yl)amino)-N-(methyl-d₃)nicotinamide (8c)

To a solution of 8b (90 mg, 0.37 mmol) and 4,6-dichloro-N-(methyl-d₃)nicotinamide (93 mg, 0.45 mmol) in THF (0.8 mL) was added LiHMDS (1.5 mL, 1.5 mmol, 1 M in THF) at −40° C. The reaction was stirred at −40° C. to r.t. for 1 h and quenched with H₂O (1 mL). The organic solvent was evaporated under reduce pressure. The formed solid was collected by filtering and was dried to afford 8c (50 mg, 38% yield) as a red solid. LC-MS (ESI, Method 3) t_{R}=1.60 min, m/z (M+H)⁺=414.2.

### Step 4. 4-((1-(2,2-Difluoropropyl)-7-methoxy-1H-pyrazolo[4,3-c]pyridin-6-yl)amino)-6-((1S,2S)-2-fluorocyclopropane-1-carboxamido)-N-(methyl-d3)nicotinamide (8)

A mixture of 8c (32 mg, 0.08 mmol), (1S, 2S)-2-fluorocyclopropane-1-carboxamide (40 mg, 0.40 mmol), BrettPhos Pd G3 (14 mg, 0.015 mmol) and Cs₂CO₃ (50 mg, 0.15 mmol) in 1,4-dioxane (0.5 mL) was stirred at 90° C. overnight under N₂ atmosphere. After cooling to r.t., the mixture was concentrated. And the residue was purified by prep-HPLC (Method A) to afford compound 8 (5.8 mg, 16% yield) as a white solid. ¹H NMR (400 MHz, DMSO-d₆) δ 11.66 (s, 1H), 10.76 (s, 1H), 9.23 (s, 1H), 8.71 (s, 1H), 8.68 (s, 1H), 8.58 (s, 1H), 8.33 (s, 1H), 5.00-4.82 (m, 3H), 3.90 (s, 3H), 2.23-2.20 (m, 1H), 1.69 (t, J=19.2 Hz, 3H), 1.65-1.60 (m, 1H), 1.18-1.23 (m, 1H). LC-MS (ESI, Method 2) t_{R}=3.46 min, m/z (M+H)⁺=481.2.

### Example 9

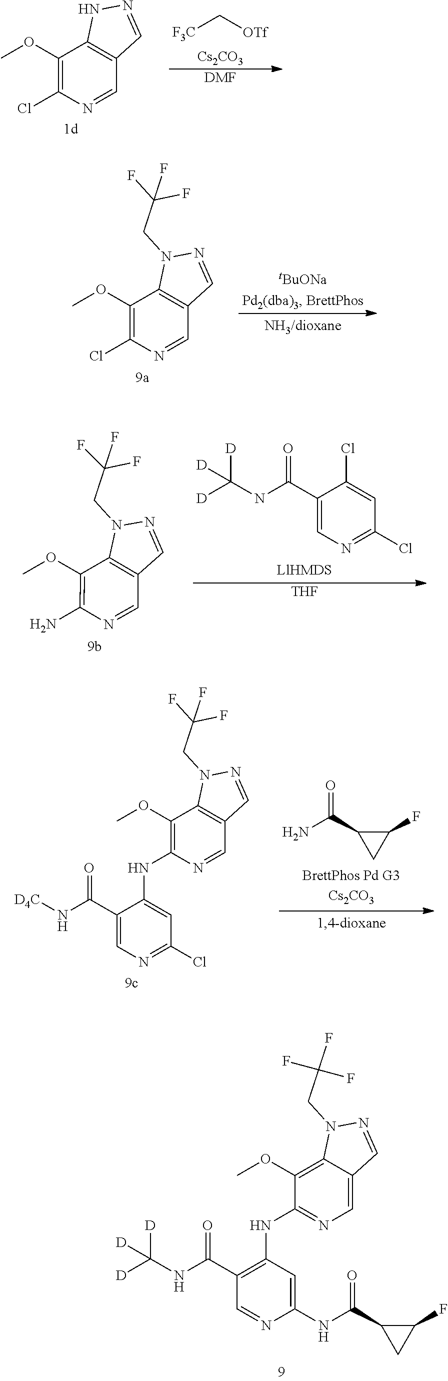

### Step 1. 6-Chloro-7-methoxy-1-(2,2,2-trifluoroethyl)-1H-pyrazolo[4,3-c]pyridine (9a)

A mixture of 1d (1 g, 5.4 mmol), 2,2,2-trifluoroethyl trifluoromethanesulfonate (2.5 g, 11 mmol) and Cs₂CO₃ (3.5 g, 11 mmol) in DMF (10 mL) was stirred at 25° C. for 1 h. The reaction mixture was diluted with H₂O (20 mL) and extracted with EtOAc (20 mL). The organic layer was washed with brine (20 mL), dried over Na₂SO₄ and filtered. The filtrate was concentrated and the residue was purified by flash chromatography on silical gel (PE/EA=3/1) to afford compound 9a (254 mg, 18% yield) as a yellow solid. LC-MS (ESI, Method 3) t_{R}=1.23 min, m/z (M+H)⁺=266.0.

### Step 2. 7-Methoxy-1-(2,2,2-trifluoroethyl)-1H-pyrazolo[4,3-c]pyridin-6-amine (9b)

A mixture of 9a (254 mg, 0.96 mmol), Pd₂(dba)₃ (17 mg, 0.019 mmol), BrettPhos (25 mg, 0.047 mmol), BuONa (137 mg, 1.4 mmol) and sat. NH₃/dioxane (4 mL) was stirred at 100° C. for 12 h. The mixture was cooled, concentrated and the residue was purified by prep-HPLC (Method A) to afford compound 9b (59 mg, 25% yield) as a white solid. LC-MS (ESI, Method 3) t_{R}=1.34 min, m/z (M+H)⁺=247.2.

### Step 3. 6-Chloro-4-((7-methoxy-1-(2,2,2-trifluoroethyl)-1H-pyrazolo[4,3-c]pyridin-6-yl)amino)-N-(methyl-d₃)nicotinamide (9c)

To a solution of 9b (45 mg, 0.18 mmol) and 4,6-dichloro-N-(methyl-d₃)nicotinamide (46 mg, 0.22 mmol) in THF (0.5 mL) was added LiHMDS (0.7 mL, 0.7 mmol, 1 M in THF) at −40° C. The reaction was stirred at −40° C. to r.t. for 1 h and quenched with H₂O (2 mL). The organic solvent was evaporated under reduced pressure. The formed solid was collected by filtering and was dried to afford compound 9c (12 mg, 16% yield) as a white solid. LC-MS (ESI, Method 3) t_{R}=1.63 min, m/z (M+H)⁺=418.2.

### Step 4. 6-((1S,2S)-2-fluorocyclopropane-1-carboxamido)-4-((7-methoxy-1-(2,2,2-trifluoroethyl)-1H-pyrazolo[4,3-c]pyridin-6-yl)amino)-N-(methyl-d₃)nicotinamide (9)

A mixture of 9c (50 mg, 0.12 mmol), (1S, 2S)-2-fluorocyclopropane-1-carboxamide (62 mg, 0.60 mmol), BrettPhos Pd G3 (22 mg, 0.024 mmol) and Cs₂CO₃ (78 mg, 0.24 mmol) in 1,4-dioxane (0.5 mL) was stirred at 90° C. overnight under N₂ atmosphere. After cooling to r.t., the mixture was concentrated. And the residue was purified by prep-HPLC (Method A) to afford compound 9 (3.3 mg, 6% yield) as a white solid. ¹H NMR (400 MHz, DMSO-d₆) δ 11.73 (s, 1H), 10.78 (s, 1H), 9.27 (s, 1H), 8.73 (s, 1H), 8.70 (s, 1H), 8.59 (s, 1H), 8.39 (s, 1H), 5.34 (q, J=8.0 Hz, 2H), 5.01-4.83 (m, 1H), 3.93 (s, 3H), 2.22-2.18 (m, 1H), 1.68-1.61 (m, 1H), 1.24-1.14 (m, 1H). LC-MS (ESI, Method 2) t_{R}=3.33 min, m/z (M+H)⁺=485.2.

### Example 10

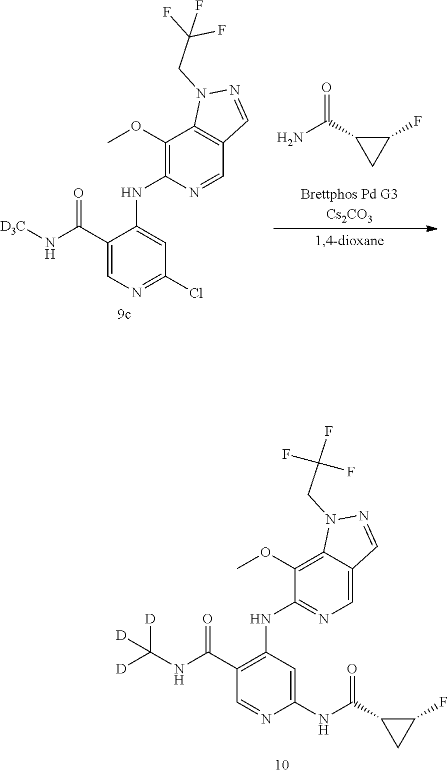 [SMILES: [2H]C([2H])([2H])NC(=O)c1cnc(Cl)cc1Nc1ncc2cnn(CC(F)(F)F)c2c1OC ; NC(=O)[C@H]1C[C@H]1F ; [2H]C([2H])([2H])NC(=O)c1cnc(NC(=O)[C@H]2C[C@H]2F)cc1Nc1ncc2cnn(CC(F)(F)F)c2c1OC]

### Step 1. 6-((1R,2R)-2-fluorocyclopropane-1-carboxamido)-4-((7-methoxy-1-(2,2,2-trifluoroethyl)-1H-pyrazolo[4,3-c]pyridin-6-yl)amino)-N-(methyl-d₃)nicotinamide (10)

A mixture of 9c (30 mg, 0.07 mmol), (1R, 2R)-2-fluorocyclopropane-1-carboxamide (37 mg, 0.36 mmol), BrettPhos Pd G3 (13 mg, 0.014 mmol) and Cs₂CO₃ (47 mg, 0.14 mmol) in 1,4-dioxane (0.5 mL) was stirred at 90° C. overnight under N₂ atmosphere. After cooling to r.t., the mixture was concentrated. And the residue was purified by prep-HPLC (Method A) to afford 10 (10 mg, 29% yield) as a white solid. ¹H NMR (400 MHz, DMSO-d₆) δ 11.73 (s, 1H), 10.78 (s, 1H), 9.27 (s, 1H), 8.74 (s, 1H), 8.70 (s, 1H), 8.60 (s, 1H), 8.39 (s, 1H), 5.34 (d, J=8.4 Hz, 2H), 5.01-4.83 (m, 1H), 3.93 (s, 3H), 2.24-2.19 (m, 1H), 1.68-1.61 (m, 1H), 1.17-1.14 (m, 1H). LC-MS (ESI, Method 2) t_{R}=3.32 min, m/z (M+H)⁺=485.2.

### Example 11

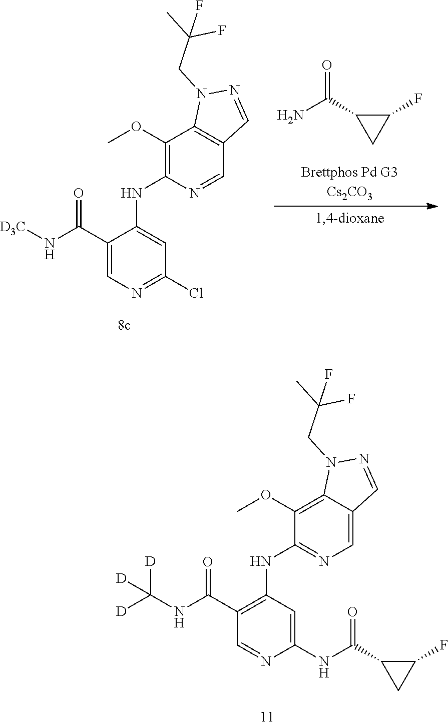 [SMILES: [2H]C([2H])([2H])NC(=O)c1cnc(Cl)cc1Nc1ncc2cnn(CC(C)(F)F)c2c1OC ; NC(=O)[C@H]1C[C@H]1F ; [2H]C([2H])([2H])NC(=O)c1cnc(NC(=O)[C@H]2C[C@H]2F)cc1Nc1ncc2cnn(CC(C)(F)F)c2c1OC]

### Step 1. 4-((1-(2,2-Difluoropropyl)-7-methoxy-1H-pyrazolo[4,3-c]pyridin-6-yl)amino)-6-((1R,2R)-2-fluorocyclopropane-1-carboxamido)-N-(methyl-d₃)nicotinamide (11)

A mixture of 8c (50 mg, 0.12 mmol), (1R, 2R)-2-fluorocyclopropane-1-carboxamide (62 mg, 0.60 mmol), BrettPhos Pd G3 (22 mg, 0.024 mmol) and Cs₂CO₃ (78 mg, 0.24 mmol) in 1,4-dioxane (0.5 mL) was stirred at 90° C. for 2 h under N₂ atmosphere. After cooling to r.t., the mixture was concentrated. And the residue was purified by prep-HPLC (Method A) to afford 11 (13 mg, 22% yield) as a white solid. ¹H NMR (400 MHz, DMSO-d₆) δ 11.66 (s, 1H), 10.75 (s, 1H), 9.23 (s, 1H), 8.71 (s, 1H), 8.67 (s, 1H), 8.58 (s, 1H), 8.33 (s, 1H), 5.01-4.80 (m, 3H), 3.90 (s, 3H), 2.22-2.17 (m, 1H), 1.69 (t, J=19.2 Hz, 3H), 1.60-1.58 (m, 1H), 1.17-1.12 (m, 1H). LC-MS (ESI, Method 2) t_{R}=2.56 min, m/z (M+H)⁺=481.2.

### Example 12

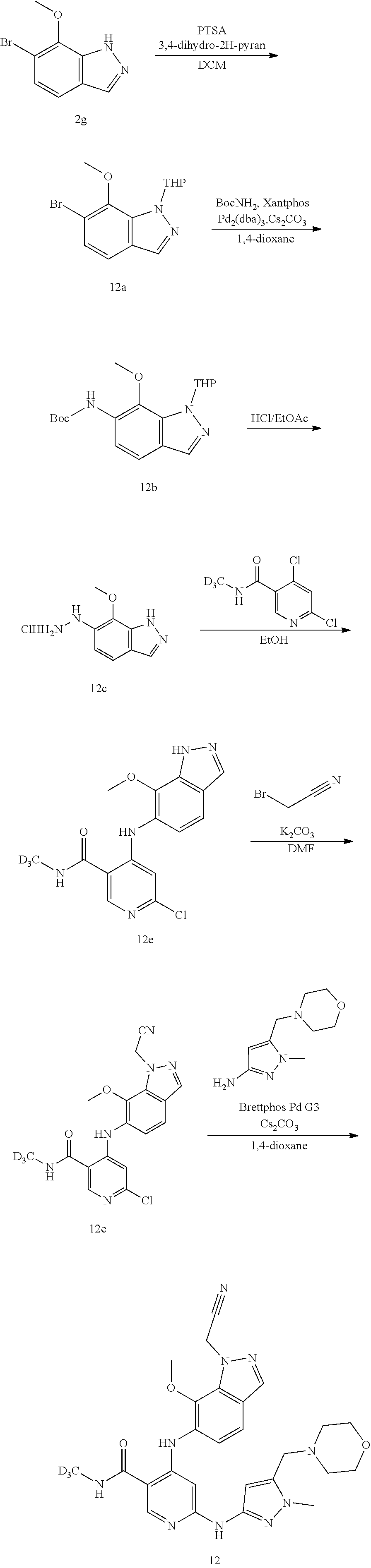

### Step 1. 6-Bromo-7-methoxy-1-(tetrahydro-2H-pyran-2-yl)-1H-indazole (12a)

To a solution of 2g (450 mg, 1.98 mmol) and 3,4-dihydro-2H-pyran (500 mg, 5.95 mmol, 0.54 mL) in DCM (5 mL) was added TsOH·H₂O (38 mg, 0.2 mmol). After stirring at 5° C. for 12 h, the reaction mixture was concentrated and the residue was purified by flash chromatography on silica gel (PE/EA=6/1) to afford 12a (450 mg, 73% yield) as a yellow solid. LC-MS (ESI, Method 3) t_{R}=1.41 min, m/z (⁷⁹Br, M+H)⁺=311.1.

### Step 2. Tert-butyl (7-methoxy-1-(tetrahydro-2H-pyran-2-yl)-1H-indazol-6-yl)carbamate (12b)

A mixture of 12a (400 mg, 1.29 mmol), Cs₂CO₃ (838 mg, 2.57 mmol), Pd₂(dba)₃ (118 mg, 0.13 mmol), BocNH₂ (301 mg, 2.57 mmol) and Xantphos (74 mg, 0.13 mmol) in 1,4-dioxane (3 mL) was stirred at 100° C. under N₂ for 12 h. The reaction mixture was concentrated and the residue was purified by flash chromatography on silica gel (PE/EA=4/1) to afford 12b (400 mg, 90% yield) as a yellow solid. LC-MS (ESI, Method 3) t_{R}=1.39 min, m/z (M−84+H)⁺=264.2.

### Step 3. 7-Methoxy-1H-indazol-6-amine hydrochloride (12c)

A solution of 12b (400 mg, 1.15 mmol) in HCl/EtOAc (4 mL, 2M) was stirred at 35° C. for 2 h. The formed solid was collected by filtering and was dried to afford 12c (200 mg, 87% yield) as a yellow solid. LC-MS (ESI, Method 3) t_{R}=0.36 min, m/z (M+H)⁺=163.9.

### Step 4. 6-Chloro-4-((7-methoxy-1H-indazol-6-yl)amino)-N-(methyl-d₃)nicotinamide (12d)

To a solution of 12c (200 mg, 1.00 mmol) and 4,6-dichloro-N-(methyl-d₃)nicotinamide (271 mg, 1.30 mmol) in EtOH (2 mL) was stirred at 80° C. for 12 h. The reaction mixture was concentrated and the residue was purified by flash chromatography on silica gel (DCM/MeOH=20/1) to afford 12d (200 mg, 60% yield) as a yellow solid. LC-MS (ESI, Method 3) t_{R}=1.06 min, m/z (M+H)⁺=335.0.

### Step 5. 6-Chloro-4-((1-(cyanomethyl)-7-methoxy-1H-indazol-6-yl)amino)-N-(methyl-d3)nicotinamide (12e)

To a solution of 12d (130 mg, 0.39 mmol) in DMF (1.5 mL) was slowly added NaH (47 mg, 1.16 mmol, 60% in mineral oil) at 0° C. and stirred at 0° C. for 30 min. Then 2-bromoacetonitrile (70 mg, 0.58 mmol, 0.04 mL) was added to the mixture at 0° C. After stirring at 25° C. for 12 h, the reaction solution was poured into ice-water (5 mL) and extracted with EtOAc (10 mL*2). The combined organic phase was washed with brine (5 mL), dried with Na₂SO₄, filtered and concentrated. The residue was purified by flash chromatography on silica gel (DCM/MeOH=30/1) to afford 12e (85 mg, 59% yield) as a yellow solid. LC-MS (ESI, Method 3) t_{R}=1.15 min, m/z (M+H)⁺=374.2.

### Step 6. 4-((1-(Cyanomethyl)-7-methoxy-1H-indazol-6-yl)amino)-N-(methyl-d₃)-6-((1-methyl-5-(morpholinomethyl)-1H-pyrazol-3-yl)amino)nicotinamide (12)

A mixture of 12e (50 mg, 0.13 mmol), 1-methyl-5-(morpholinomethyl)pyrazol-3-amine (32 mg, 0.16 mmol, CAS: 1328640-42-1), Cs₂CO₃ (87 mg, 0.27 mmol) and BrettPhos Pd G3 (24 mg, 0.027 mmol) in 1,4-dioxane (0.5 mL) was stirred at 110° C. under N₂ atmosphere for 12 h. The reaction mixture was concentrated and the residue was purified by flash chromatography on silica gel (DCM/MeOH=10/1) to afford 12 (17.5 mg, 25% yield) as a yellow solid. ¹H NMR (400 MHz, DMSO-d₆) δ 10.65 (s, 1H), 9.20 (s, 1H), 8.43 (s, 1H), 8.41 (s, 1H), 8.20 (s, 1H), 7.61 (d, J=8.8 Hz, 1H), 7.37 (d, J=8.4 Hz, 1H), 7.25 (s, 1H), 6.04 (s, 1H), 5.75 (s, 2H), 3.87 (s, 3H), 3.61 (s, 3H), 3.55-3.53 (m, 4H), 3.42 (s, 2H), 2.34-2.32 (m, 4H). LC-MS (ESI, Method 2) t_{R}=1.99 min, m/z (M+H)⁺=534.3.

### Example 13

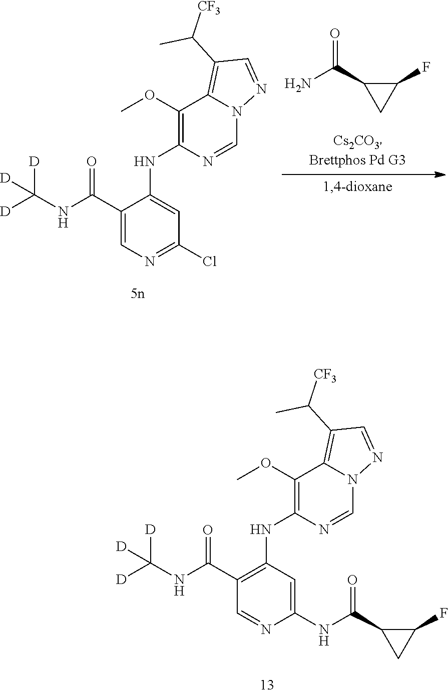

### Step 1. 6-((1S,2S)-2-fluorocyclopropane-1-carboxamido)-4-((4-methoxy-3-(1,1,1-trifluoropropan-2-yl)pyrazolo[1,5-c]pyrimidin-5-yl)amino)-N-(methyl-d₃)nicotinamide (13)

A mixture of 5n (10 mg, 0.023 mmol), (1S,2S)-2-fluorocyclopropane-1-carboxamide (12 mg, 0.12 mmol), BrettPhos Pd G3 (4 mg, 0.005 mmol) and Cs₂CO₃ (15 mg, 0.05 mmol) in 1,4-dioxane (0.5 mL) was stirred at 90° C. overnight under N₂ atmosphere. After cooling to r.t., the mixture was concentrated. And the residue was purified by prep-HPLC (Method A) to afford 13 (0.6 mg, 5% yield) as a white solid. ¹H NMR (400 MHz, DMSO-d₆) δ 11.58 (s, 1H), 10.83 (s, 1H), 9.37 (s, 1H), 8.89 (s, 1H), 8.73 (s, 1H), 8.61 (s, 1H), 8.28 (s, 1H), 5.01-4.83 (m, 1H), 4.13-4.11 (m, 1H), 3.90 (s, 3H), 2.21-2.18 (m, 1H), 1.56 (d, J=6.4 Hz, 3H), 1.18-1.16 (m, 1H), 1.09-1.05 (m, 1H). LC-MS (ESI, Method 2) t_{R}=3.24 min, m/z (M+H)⁺=499.0.

### Example 14

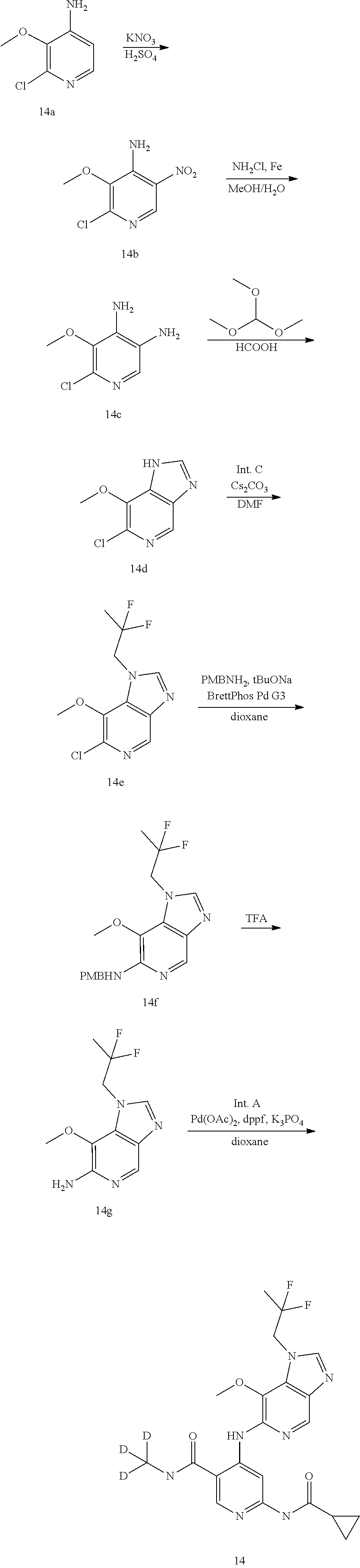

### Step 1. 2-Chloro-3-methoxy-5-nitropyridin-4-amine (14b)

To a solution of 14a (1.0 g, 6.31 mmol) in conc. H₂SO₄ (10 mL) was added KNO₃ (701 mg, 6.94 mmol) at 0° C., then the mixture was stirred at r.t. for 4 h. The mixture was poured into ice-water, basified to pH=13 with NH₃·H₂O, extracted with EtOAc (50 mL*5), washed with brine (50 mL), dried over Na₂SO₄, concentrated and purified by flash chromatography (PE/EA=10/1 to 1/1) to get compound 14b (800 mg, 62% yield) as a pale yellow solid. LC-MS (ESI, Method 4) t_{R}=1.88 min, m/z (M+H)⁺=204.1.

### Step 2. 6-Chloro-5-methoxypyridine-3,4-diamine (14c)

To a solution of 14b (350 mg, 1.72 mmol) in MeOH (4 mL) and H₂O (1 mL) was added Fe powder (480 mg, 8.60 mmol) and NH₄Cl (460 mg, 8.6 mmol). Then the mixture was stirred at 70° C. for 2 h. The mixture was filtered and washed with EtOAc (10 mL). Then the filtrate was concentrated and diluted with H₂O (10 mL), extracted with EtOAc (10 mL*3), washed with brine (10 mL), dried over Na₂SO₄, concentrated and purified by flash chromatography (DCM/MeOH=50/1 to 5/1) to get compound 14c (290 mg, 97% yield) as a yellow oil. LC-MS (ESI, Method 4) t_{R}=0.46 min, m/z (M+H)⁺=174.1.

### Step 3. 6-Chloro-7-methoxy-1H-imidazo[4,5-c]pyridine (14d)

To a solution of 14c (290 mg, 1.67 mmol) in trimethoxymethane (1.77 g, 16.71 mmol, 1.83 mL) was added formic acid (80 mg, 1.67 mmol), then the mixture was stirred at 100° C. for 1 h. The mixture was concentrated and diluted with H₂O (20 mL), extracted with EtOAc (15 mL*3), washed with brine (15 mL), dried over Na₂SO₄, concentrated to get compound 14d (300 mg, 98% yield) as a yellow solid. LC-MS (ESI, Method 4) t_{R}=0.88 min, m/z (M+H)⁺=184.1.

### Step 4. 6-Chloro-1-(2,2-difluoropropyl)-7-methoxy-1H-imidazo[4,5-c]pyridine (14e)

To a solution of 14d (300 mg, 1.63 mmol) in DMF (5 mL) was added Cs₂CO₃ (1.60 g, 4.90 mmol) and Int. C (559 mg, 2.45 mmol). Then the mixture was stirred at r.t. for 4 h. Then the mixture was diluted with H₂O (30 mL), extracted with EA (20 mL*3), washed with brine (20 mL), dried over Na₂SO₄, concentrated and purified by prep-TLC (DCM/MeOH=30/1) to get compound 14e (120 mg, 28% yield) as a yellow solid. ¹H NMR (400 MHz, CDCl₃) δ 8.68 (s, 1H), 7.94 (s, 1H), 4.74 (t, J=13.2 Hz, 2H), 4.07 (s, 3H), 1.71 (t, J=18.4 Hz, 3H). LC-MS (ESI, Method 4) t_{R}=2.16 min, m/z (M+H)⁺=262.1.

### Step 5. 1-(2,2-Difluoropropyl)-7-methoxy-N-(4-methoxybenzyl)-1H-imidazo[4,5-c]pyridin-6-amine (14f)

A mixture of 14e (120 mg, 0.46 mmol), (4-methoxyphenyl)methanamine (126 mg, 0.92 mmol), BrettPhos Pd G3 (83 mg, 0.091 mmol), tBuONa (88 mg, 0.92 mmol) in THF (1.5 mL) was stirred at 90° C. for 2 h. The mixture was concentrated and purified by flash chromatography (DCM/MeOH=100/1 to 20/1) to get compound 14f (150 mg, 90% yield) as a yellow oil. LC-MS (ESI, Method 4) t_{R}=1.98 min, m/z (M+H)⁺=363.2.

### Step 6. 1-(2,2-Difluoropropyl)-7-methoxy-1H-imidazo[4,5-c]pyridin-6-amine (14g)

A solution of 14f (150 mg, 0.41 mmol) in TFA (2 mL) was stirred at r.t. for 2 h. The mixture was concentrated and diluted with H₂O (20 mL), adjusted to pH >7 with aq. Na₂CO₃ and extracted with EtOAc (20 mL*3). The combined organic layer was washed with brine (30 mL*2), dried over Na₂SO₄, concentrated and purified by flash chromography (DCM/MeOH=100/1 to 5/1) to get compound 14g (90 mg, 90% yield) as a yellow solid. LC-MS (ESI, Method 4) t_{R}=0.50 min, m/z (M+H)⁺=243.1.

### Step 7. 6-(Cyclopropanecarboxamido)-4-((1-(2,2-difluoropropyl)-7-methoxy-1H-imidazo[4,5-c]pyridin-6-yl)amino)-N-(methyl-d₃)nicotinamide (14)

A mixture of 14g (2 mg, 0.008 mmol), Int. A (2 mg, 0.008 mmol), Pd(OAc)₂ (0.9 mg, 0.004 mmol), dppf (4 mg, 0.008 mmol), K₃PO₄ (4 mg, 17 mmol) in dioxane (0.5 mL) was stirred at 100° C. for 16 h. The mixture was concentrated and purified by prep-HPLC (Method E) to get compound 14 (1.0 mg, 26% yield) as a pale yellow solid. ¹H NMR (400 MHz, DMSO-d₆) δ 11.46 (s, 1H), 10.65 (s, 1H), 9.09 (s, 1H), 8.61 (s, 1H), 8.51 (s, 1H), 8.48 (s, 1H), 8.18 (s, 1H), 4.82 (t, J=14.4 Hz, 2H), 3.84 (s, 3H), 1.97-1.95 (m, 1H), 1.71 (t, J=19.2 Hz, 3H), 0.77-0.73 (m, 4H). LC-MS (ESI, Method 4) t_{R}=2.01 min, m/z (M+H)⁺=463.3.

### Example 15

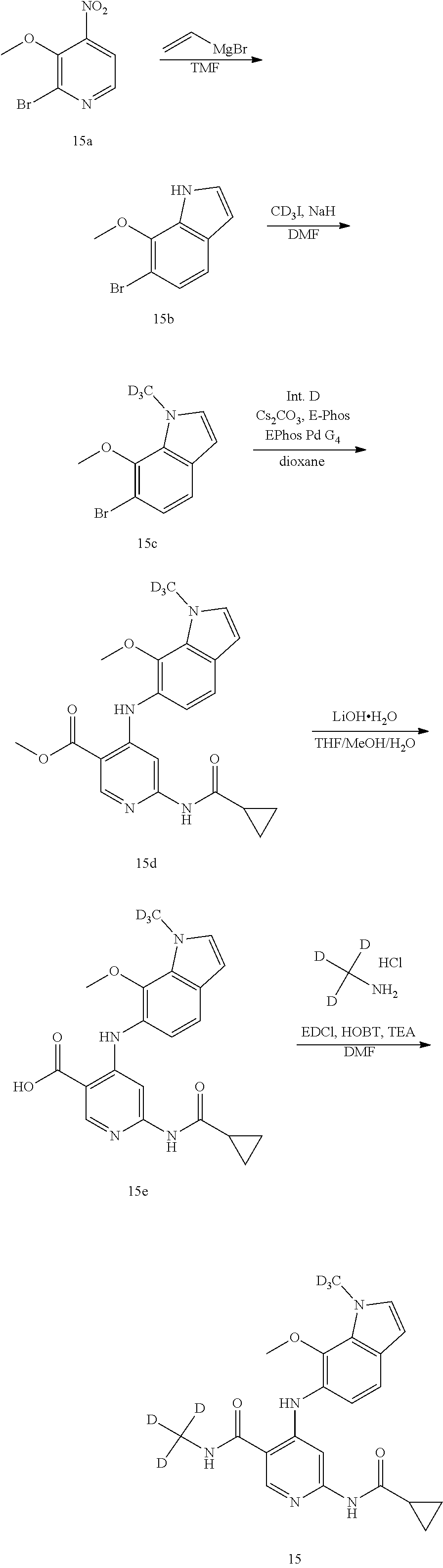

### Step 1. 6-Bromo-7-methoxy-1H-indole (15b)

To a solution of 15a (17 g, 219.9 mmol) in THF (100 mL) at −40° C. under N₂ atmosphere was added vinylmagnesium bromide (220 mL, 220 mmol, 1 M in THF). The resulting mixture was stirred at −20° C. for 30 min. The reaction was then quenched with aq. NH₄Cl (300 mL) and extracted with EA (300 mL*3). The combined organic layer was washed by brine (300 mL*2), dried over sodium sulphate and evaporated in vacuo. The residue was purified by flash chromatography (EA in PE is 5-10%) to give 15b (4.78 g, 20% yield) as a yellow oil. LC-MS (ESI, Method 2), t_{R}=1.88 min, m/z (⁷⁹Br, M+H)⁺=225.9.

### Step 2. 6-Bromo-7-methoxy-1-(methyl-d3)-1H-indole (15c)

To a solution of 15b (4.5 g, 19.9 mmol) in DMF (50 mL) at 0° C. under N₂ atmosphere was added NaH (1.19 g, 29.85 mmol, 60% in mineral oil). The resulting mixture was stirred at 0° C. for 30 min and added iodomethane-d3 (3.17 g, 21.89 mmol). The resulting mixture was stirred at 25° C. for 16 h. The reaction was then quenched with aq. NH₄Cl (50 mL) and extracted with EA (200 mL*3). The combined organic layer was washed by brine (200 mL*2), dried over sodium sulphate and evaporated in vacuo. The residue was purified by flash chromatography (EA in PE is 5-15%) to give 15c (4.57 g, 68% yield) as a yellow oil. LC-MS (ESI, Method 2), t_{R}=2.19 min, m/z (⁷⁹Br, M+H)⁺=243.0.

### Step 3. Methyl 6-(cyclopropanecarboxamido)-4-((7-methoxy-1-(methyl-d3)-1H-indol-6-yl)amino)nicotinate (15d)

To a solution of 15c (400 mg, 1.64 mmol) in dioxane (6 mL) was added Int. D (402 mg, 1.15 mmol), Cs₂CO₃ (1.61 g, 4.94 mmol), EPhos (87.9 mg, 0.164 mmol) and EPhos Pd G₄ (221.4 mg, 0.25 mmol). The reaction mixture was stirred at 90° C. under N₂ atmosphere for 16 h. The mixture was cooled to room temperature and diluted with water (30 mL), extracted with EA (30 mL*3). The combined organic layer was washed by brine (50 mL*2), dried over sodium sulphate, evaporated in vacuo and purified by prep-TLC (DCM/MeOH=20/1) to give 15d (120 mg, 11% yield) as a yellow solid. LC-MS (ESI, Method 2), t_{R}=1.76 min, m/z (M+H)⁺=398.2.

### Step 4. 6-(Cyclopropanecarboxamido)-4-((7-methoxy-1-(methyl-d₃)-1H-indol-6-yl)amino)nicotinic acid (15e)

To a solution of 15d (110 mg, 0.28 mmol) in THF/MeOH/H₂O=3/2/1 (6 mL) was added LiOH·H₂O (23.2 mg, 0.55 mmol) and stirred at 40° C. under N₂ atmosphere for 16 h. The mixture was diluted with water (20 mL) and extracted with EA (20 mL*3). The combined organic layer was washed by brine (20 mL*2), dried over sodium sulphate, evaporated in vacuo and purified by prep-TLC (DCM/MeOH=10/1) to give 15e (85 mg, 69% yield) as a yellow solid. LC-MS (ESI, Method 2), t_{R}=1.49 min, m/z (M+H)⁺=384.0.

### Step 5. 6-(Cyclopropanecarboxamido)-4-((7-methoxy-1-(methyl-d3)-1H-indol-6-yl)amino)-N-(methyl-d₃)nicotinamide (15)

To a solution of 15e (85 mg, 0.22 mmol) in DMF (2 mL) was added CD₃NH₂·HCl (23.5 mg, 0.33 mmol), EDCl (51.1 mg, 0.266 mmol), HOBt (36 mg, 0.266 mmol) and TEA (112 mg, 1.11 mmol, 0.15 mL). The reaction mixture was stirred at 25° C. under N₂ atmosphere for 16 h. The mixture was diluted with water (20 mL) and extracted with EA (10 mL*3). The combined organic layer was washed by brine (10 mL*2), dried over sodium sulphate, evaporated in vacuo and purified by prep-TLC (DCM/MeOH=10/1) to give 15 as a crude (26 mg). Then the crude product was purified by prep-HPLC (Method F) to give 15 (5.6 mg, 6% yield) as a yellow solid. ¹H NMR (400 MHz, DMSO-d₆) δ 10.59 (s, 1H), 10.34 (s, 1H), 8.52-8.48 (m, 2H), 7.71 (s, 1H), 7.27-7.25 (m, 2H), 6.95 (d, J=8.4 Hz, 1H), 6.40 (s, 1H), 3.73 (s, 3H), 1.94-1.92 (m, 1H), 0.72-0.69 (m, 4H). LC-MS (ESI, Method 4), t_{R}=2.49 min, m/z (M+H)⁺=400.1.

### Example 16

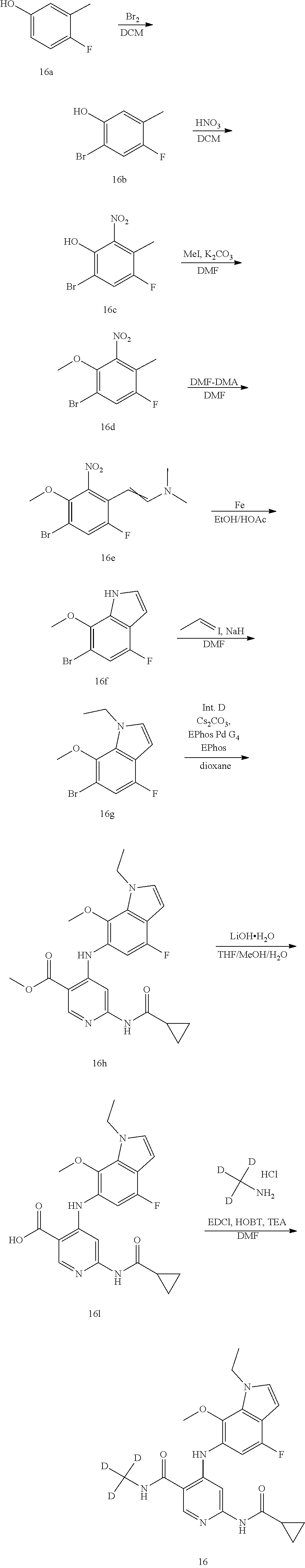

### Step 1. 2-Bromo-4-fluoro-5-methylphenol (16b)

To a solution of 16a (14 g, 79.3 mmol) in DCM (140 mL) was added Bri (15.21 g, 95.1 mmol, 4.89 mL) under nitrogen at 25° C. The reaction mixture was stirred at 25° C. for 16 h. The reaction was quenched by water (100 mL) and extracted with EA (100 mL*2), washed with water (100 mL*2) and brine (100 mL*2), dried over Na₂SO₄ and evaporated in vacuo. The crude product was purified by flash chromatography (PE) to give 16b (14 g, 73% yield) as a yellow oil. ¹H NMR (400 MHz, CDCl₃) δ 7.12 (d, JC 8.4 Hz, 1H), 6.84 (d, J 6.8 Hz, 1H), 2.20 (s, 3H).

### Step 2. 6-Bromo-4-fluoro-3-methyl-2-nitrophenol (16c)

To a solution of 16b (14 g, 68.3 mmol) in DCM (150 mL) was added nitric acid (3.2 mL, 81.9 mmol, 68%) under nitrogen at −20′° C. The reaction mixture was stirred at −20° C. for 2 h. The reaction was quenched with H₂O (50 mL) and extracted with EA (200 mL*3). The combined organic layer was washed by brine (200 mL*2), dried over Na₂SO₄ and evaporated in vacuo. The residue was purified by flash chromatography (PE) to give 16c (9.5 g, 55% yield) as a yellow solid. ¹H NMR (400 MHz, CDCl₃) δ 7.33 (s, 1H), 2.48 (s, 3H). LC-MS (ESI, Method 2), t_{R}=2.58 min, m/z (⁷⁹Br, M+H)⁺=250.0.

### Step 3. 1-Bromo-5-fluoro-2-methoxy-4-methyl-3-nitrobenzene (16d)

To a solution of 16c (5 g, 20.0 mmol) in DCM (50 mL) stirred under nitrogen at 25° C. was added a solution of CH₃I (3.41 g, 0.024 mol, 1.49 mL) and K₂CO₃ (8.29 g, 60.0 mmol). The reaction mixture was stirred at 25° C. for 16 h. The reaction was then quenched by water (100 mL), extracted with ethyl acetate (100 mL), washed with water (100 mL*2) and brine (100 mL*2), dried over anhydrous sodium sulfate and concentrated under vacuum. The crude product was purified by flash chromatography (EA in PE is 10-30%) to afford 16d (4.9 g, 92% yield) as a yellow oil. ¹H NMR (400 MHz, CDCl₃) δ 7.39 (d, J=8.4 Hz, 1H), 3.93 (s, 3H), 2.18 (s, 3H).

### Step 4. 2-(4-Bromo-6-fluoro-3-methoxy-2-nitrophenyl)-N,N-dimethylethen-1-amine (16e)

To a solution of 16d (2 g, 7.6 mmol) in DMF (9 mL) was added DMF-DMA (20 mL, 22.8 mmol) under nitrogen atmosphere at 25° C. The resulting solution was stirred at 110° C. for 3 h under nitrogen atmosphere. The solvent was evaporated under vacuum to give 16e (2.1 g, crude) as a yellow oil, which was used for the next step directly without further purification.

### Step 5. 6-Bromo-4-fluoro-7-methoxy-1H-indole (16f)

To a solution of 16e (2 g, 6.3 mmol) in EtOH/HOAc=1/1 (40 mL) was added Fe powder (1.06 g, 18.9 mmol) under nitrogen at 25° C. The reaction mixture was stirred at 80° C. for 2 h. The reaction was then quenched by water (50 mL), extracted with EtOAc (100 mL*2), washed with water (100 mL*2) and brine (100 mL*2), dried over anhydrous sodium sulfate and concentrated under vacuum. The crude product was purified by flash chromatography (EA in PE is 10-40%) to afford 16f (550 mg, 33% yield) as a yellow oil. LC-MS (ESI, Method 4), t_{R}=2.75 min, m/z (⁷⁹Br, M−H)⁻=242.0.

### Step 6. 6-Bromo-1-ethyl-4-fluoro-7-methoxy-1H-indole (16g)

To a solution of 16f (700 mg, 2.88 mmol) in DMF (10 mL) stirred under nitrogen at 0° C. was added NaH (127 mg, 3.17 mmol, 60% in mineral oil). The reaction mixture was stirred at 0° C. for 0.5 h. To the mixture was added C₂H₅I (494 mg, 3.17 mmol, 2.5 mL). The mixture was stirred at 70° C. for 16 h. After cooling to room temperature, the reaction was quenched by water (50 mL), extracted with EA (100 mL*2), washed with water (100 mL*2) and brine (100 mL*2), dried over anhydrous sodium sulfate and concentrated under vacuum. The residue was purified by flash chromatography (EA in PE is 10-30%) to give 16g (500 mg, 64% yield) as a yellow solid. ¹H NMR (400 MHz, DMSO-d₆) δ 7.46 (d, J=3.2 Hz, 1H), 7.04 (d, J=9.2 Hz, 1H), 6.53 (d, J=3.2 Hz, 1H), 4.35 (q, J=7.2 Hz, 2H), 3.86 (s, 3H), 1.35 (t, J=7.2 Hz, 3H).

### Step 7. Methyl 6-(cyclopropanecarboxamido)-4-((1-ethyl-4-fluoro-7-methoxy-1H-indol-6-yl)amino)nicotinate (16h)

To a solution of 16g (500 mg, 1.84 mmol) in dioxane (5 mL) was added Cs₂CO₃ (599 mg, 5.52 mmol), EPhos Pd G4 (169 mg, 0.18 mmol), EPhos (196 mg, 0.37 mmol) and Int. D (642 mg, 1.84 mmol). The reaction mixture was stirred at 100° C. for 16 h under N₂ atmosphere. The mixture was cooled to room temperature, diluted with water (10 mL) and extracted with EtOAc (15 mL*3). The combined organic layer was washed by brine (20 mL*2), dried over sodium sulphate and evaporated in vacuo. The residue was purified by prep-TLC (DCM/MeOH=20/1) to give 16h (180 mg, 22% yield) as a yellow solid. LC-MS (ESI, Method 2), t_{R}=1.74 min, m/z (M+H)⁺=427.1.

### Step 8. 6-(Cyclopropanecarboxamido)-4-((1-ethyl-4-fluoro-7-methoxy-1H-indol-6-yl)amino)nicotinic acid (16i)

To a solution of 16h (180 mg, 0.42 mmol) in THF (1.2 mL), MeOH (0.6 mL) and H₂O (0.4 mL) was added LiOH·H₂O (36 mg, 0.846 mmol) and stirred at 40° C. for 16 h under N₂ atmosphere. The mixture was diluted with water (5 mL), adjusted to pH=2 with 2 N HCl and extracted with EtOAc (5 mL*3). The combined organic layer was washed by brine (5 mL*2), dried over sodium sulphate and evaporated in vacuo. The residue was purified by prep-TLC (DCM/MeOH=10/1) to give 16i (150 mg, 86% yield) as a yellow solid. LC-MS (ESI, Method ²), t_{R}=1.61 min, m/z (M+H)⁺=413.1.

### Step 9. 6-(Cyclopropanecarboxamido)-4-((1-ethyl-4-fluoro-7-methoxy-1H-indol-6-yl)amino)-N-(methyl-d₃)nicotinamide (16)

To a solution of 16i (150 mg, 0.35 mmol) in DMF (2 mL) was added CD₃NH₂·HCl (39 mg, 0.53 mmol), EDCl (82 mg, 0.42 mmol), HOBt (60 mg, 0.42 mmol) and TEA (221 mg, 2.1 mmol, 0.3 mL). The reaction mixture was stirred at 25° C. for 16 h under N₂ atmosphere. The mixture was diluted with water (5 mL) and extracted with EtOAc (5 mL*3). The combined organic layer was washed by brine (5 mL*2), dried over sodium sulphate, evaporated in vacuo and purified by prep-TLC (DCM/MeOH=10/1) to give the crude product (30 mg). Then the crude product was purified by prep-HPLC (Method F) to give 16 (10 mg, 6% yield) as a yellow solid. ¹H NMR (400 MHz, DMSO-d₆) δ 10.66 (s, 1H), 10.42 (s, 1H), 8.56 (s, 1H), 8.50 (s, 1H), 7.77 (s, 1H), 7.40 (d, J=3.2 Hz, 1H), 6.78 (d, J=10.8 Hz, 1H), 6.48 (d, J=3.2 Hz, 1H), 4.35 (q, J=7.2 Hz, 2H), 3.71 (s, 3H), 1.94-1.93 (m, 1H), 1.35 (t, J=7.2 Hz, 3H), 0.73-0.71 (m, 4H). LC-MS (ESI, Method 4), t_{R}=3.07 min, m/z (M+H)⁺=429.1.

### Example 17

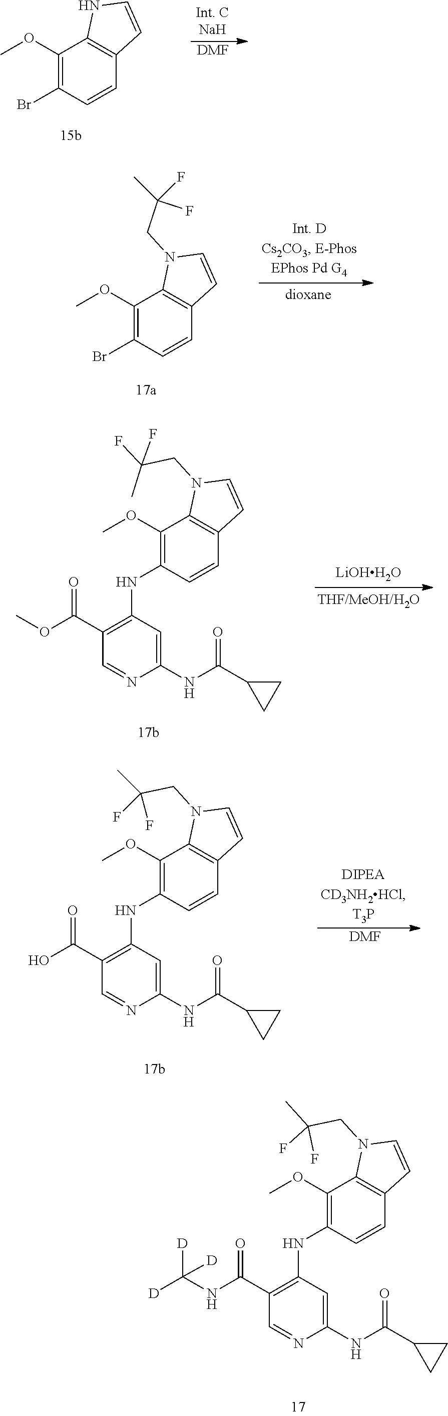

### Step 1. 6-Bromo-1-(2,2-difluoropropyl)-7-methoxy-1H-indole (17a)

To a solution of 15b (1.5 g, 6.6 mmol) in DMF (15 mL) was added NaH (400 mg, 9.9 mmol, 60% in mineral oil) and Int. C (700 mg, 9.9 mmol) at 0° C. The reaction mixture was stirred at 25° C. for 3 h under N₂ atmosphere. The reaction mixture was diluted with water (100 mL), then extracted with EtOAc (50 mL*2). The combined organic layer was washed with water (50 mL*2), brine (50 mL*2), dried over anhydrous Na₂SO₄, filtered and concentrated. The residue was purified by flash chromatography (EA in PE is 10-30%) to give 17a (830 mg, 37% yield) as a yellow solid. LC-MS ((ESI, Method 2), t_{R}=2.24 min, m/z (⁷⁹Br, M+H)⁺=303.8.

### Step 2. Methyl 6-(cyclopropanecarboxamido)-4-((1-(2,2-difluoropropyl)-7-methoxy-1H-indol-6-yl)amino)nicotinate (17b)

To a solution of 17a (800 mg, 2.63 mmol) in dioxane (6 mL) was added Int. D (918 mg, 2.63 mmol), Cs₂CO₃ (2.57 g, 7.89 mmol), EPhos (280.9 mg, 0.526 mmol) and EPhos Pd G4 (241.6 mg, 0.263 mmol). The reaction mixture was stirred at 90° C. under N₂ atmosphere for 16 h. The mixture was cooled to room temperature and diluted with water (30 mL), extracted with EA (50 mL*3). The combined organic layer was washed by brine (50 mL*2), dried over sodium sulphate and evaporated in vacuo. The residue was purified by prep-TLC (DCM/MeOH=20/1) to give 17b (120 mg, 6% yield) as a yellow solid. LC-MS (ESI, Method 2), t_{R}=1.65 min, m/z (M+H)⁺=459.1.

### Step 3. 6-(Cyclopropanecarboxamido)-4-((1-(2,2-difluoropropyl)-7-methoxy-1H-indol-6-yl)amino)nicotinic acid (17c)

To a solution of 17b (110 mg, 0.239 mmol) in THF/MeOH/H₂O=3/2/1 (2 mL) was added LiOH·H₂O (20.1 mg, 0.479 mmol). The resulting mixture was stirred at 40° C. for 16 h under N₂ atmosphere. The mixture was diluted with water (20 mL) and extracted with EA (2 mL*3). The combined organic layer was washed by brine (2 mL*2), dried over Na₂SO₄ and evaporated in vacuo. The residue was purified by flash chromatography (MeOH in DCM is 10-50%) to give 17c (55 mg, 41% yield) as a yellow solid. LC-MS (ESI, Method 2), t_{R}=1.59 min, m/z (M+H)⁺=445.0.

### Step 4. 6-(Cyclopropanecarboxamido)-4-((1-(2,2-difluoropropyl)-7-methoxy-1H-indol-6-yl)amino)-N-(methyl-d₃)nicotinamide (17)

A mixture of CD₃NH₂—HCl (16.4 mg, 0.234 mmol), 7 (35 mg, 0.078 mmol), DIPEA (30.5 mg, 0.236 mmol, 0.158 mL) and T₃P (148.8 mg, 0.234 mmol, 50% purity in EtOAc) in DMF (2 mL) was stirred at 70° C. for 12 h. A yellow solution was formed. The reaction mixture was diluted with water (10 mL), then extracted with EtOAc (1 mL*2). The combined organic layer was washed with water (1 mL) and brine (1 mL), dried over anhydrous Na₂SO₄, filtered and concentrated. The residue was purified by prep-HPLC (Method E) to give 17 (3.1 mg, 8% yield) as a yellow solid. ¹H NMR (400 MHz, DMSO-d₆) δ 10.62 (s, 1H), 10.34 (s, 1H), 8.55 (s, 1H), 8.49 (s, 1H), 7.69 (s, 1H), 7.32 (d, J=8.0 Hz, 1H), 7.27 (d, J=3.2 Hz, 1H), 6.98 (d, J=8.4 Hz, 1H), 6.53 (d, J=3.2 Hz, 1H), 4.81 (q, J=14.4 Hz, 2H), 3.72 (s, 3H), 1.92-1.90 (m, 1H), 1.58 (t, J=18.8 Hz, 3H), 0.70-0.72 (m, 4H). LC-MS (ESI, Method 4), t_{R}=2.79 min, m/z (M+H)⁺=461.1.

### Example 18

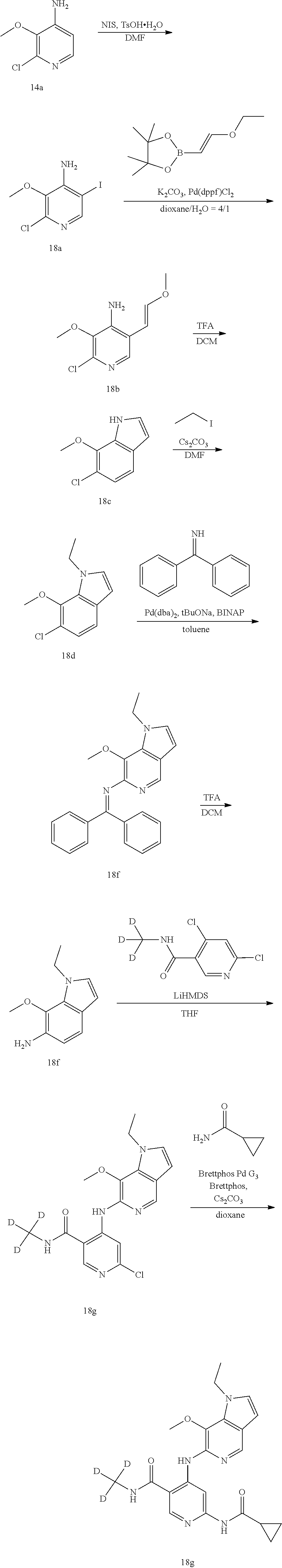

### Step 1. 2-Chloro-5-iodo-3-methoxypyridin-4-amine (18a)

To a solution of 14a (2 g, 12.6 mmol) in DMF (10 mL) was added NIS (4.25 g, 18.9 mmol) and TsOH·H₂O (240 mg, 1.2 mmol). The mixture was stirred for 12 h at 70° C. The mixture was quenched with water (50 mL), extracted with EA (50 mL*3), dried over Na₂SO₄ and concentrated to give crude product 18a. The residue was purified by flash chromatography (EA in PE is 5-15%) to give 18a (3.4 g, 85% yield) as a yellow solid. ¹H NMR (400 MHz, DMSO-d₆), 8.06 (s, 1H), 6.33 (s, 2H), 3.70 (s, 3H). LC-MS (ESI, Method 2), t_{R}=1.52 min, m/z (M+H)⁺=284.9.

### Step 2. (E)-2-chloro-5-(2-ethoxyvinyl)-3-methoxypyridin-4-amine (18b)

To a solution of 18a (3.4 g, 12 mmol) in dioxane/H₂O=4/1 (14 mL) was added (E)-2-(2-ethoxyvinyl)-4,4,5,5-tetramethyl-1,3,2-dioxaborolane (2.85 g, 14.4 mmol), K₂CO₃ (4.98 g, 3.6 mmol) and Pd(dppf)Cl₂ (1.76 g, 2.4 mmol). The mixture was stirred for 16 h at 60° C. The mixture was quenched with water (50 mL), extracted with EtOAc (50 mL*3), dried over Na₂SO₄ and concentrated to give crude product 18b. The residue was purified by flash chromatography (EA in PE is 10-20%) to give 18b (2.56 g, 88% yield) as a yellow oil. ¹H NMR (400 MHz, CDCl₃) δ 7.74 (s, 1H), 6.75 (d, J=12.8 Hz, 1H), 5.51 (d, J=12.8 Hz, 1H), 4.44 (s, 2H), 3.92 (q, J=7.2 Hz, 3H), 3.85 (s, 3H), 1.35 (t, J=7.2 Hz, 3H). LC-MS (ESI, Method 2), t_{R}=1.27 min, m/z (M+H)⁺=229.1.

### Step 3. 6-Chloro-7-methoxy-1H-pyrrolo[3,2-c]pyridine (18k)

To a solution of 18b (2.568 g, 11.2 mmol) in DCM (20 mL) was added TFA (5 mL). The mixture was stirred for 2 h at 25′° C. The mixture was concentrated. The residue was dissolved with water, adjusted to pH=7 with aq. NaHCO₃, extracted with DCM (10 mL*3), dried over Na₂SO₄ and concentrated to give 18c (2 g, crude) as a yellow solid, which was used for the next step directly without further purification. LC-MS (ESI, Method 2), t_{R}=0.20 min, m/z (M+H)⁺=183.0.

### Step 4. 6-Chloro-1-ethyl-7-methoxy-1H-pyrrolo[3,2-c]pyridine (18d)

To a solution of 18c (1 g, 5.50 mmol) and iodoethane (1.29 g, 8.25 mmol) in DMF (10 mL) stirred at 25° C. was added Cs₂CO₃(1.52 g, 11 mmol). The reaction mixture was stirred at 70° C. for 16 h. 30 mL of water was added to the mixture. The mixture was extracted with EtOAc (50 mL*3). The combined organic phase was washed with brine (50 mL), dried over Na₂SO₄ and concentrated. The residue was purified by flash chromatography (EA in PE is 10-30%) to give 18d (650 mg, crude), which was used for the next step directly without further purification. LC-MS (ESI, Method 2), t_{R}=1.54 min, m/z (M+H)⁺=211.0.

### Step 5. N-(1-ethyl-7-methoxy-1H-pyrrolo[3,2-c]pyridin-6-yl)-1,1-diphenylmethanimine (18e)

To a solution of 18d (600 mg, 2.85 mmol), diphenylmethanimine (1.55 g, 8.54 mmol) and BINAP (709.4 mg, 1.14 mmol) in toluene (15 mL) stirred at 25° C. was added Pd₂(dba)₃ (522.1 mg, 0.57 mmol) and t-BuONa (821.1 mg, 8.54 mmol). The reaction mixture was stirred at 110° C. for 16 h. Water (40 mL) was added to the mixture. The mixture was extracted with DCM (30 mL*3). The combined organic phase was washed with brine (10 mL), dried over Na₂SO₄ and concentrated. The residue was purified by prep-TLC (PE/EA=3/1) to give 18e (500 mg, 46% yield) as a yellow solid. LC-MS (ESI, Method 2), t_{R}=1.65 min m/z (M+H)⁺=356.1.

### Step 6. 1-Ethyl-7-methoxy-1H-pyrrolo[3,2-c]pyridin-6-amine (18f)

To a solution of 18e (500 mg, 1.41 mmol) in DCM (10 mL) stirred at 25° C. was added TFA (3 mL). The reaction mixture was stirred at 25° C. for 16 h. The reaction was quenched with aq. NaHCO₃ (15 mL), extracted with DCM (30 mL*3). The combined organic layer was washed by brine (5 mL), dried over sodium sulphate and evaporated in vacuo. The residue was purified by prep-TLC with (DCM/MeOH=10/1) to give 18f (170 mg, 61% yield) as a yellow solid. ¹H NMR (400 MHz, DMSO-d₆) δ 8.06 (s, 1H), 7.16 (d, J=3.2 Hz, 1H), 6.39 (d, J=3.2 Hz, 1H), 4.25 (q, J=7.2 Hz, 2H), 3.75 (s, 3H), 1.33 (t, J=7.2 Hz, 3H). LC-MS (ESI, Method 2), t_{R}=1.13 min, m/z (M+H)⁺=192.1.

### Step 7. 6-Chloro-4-((1-ethyl-7-methoxy-1H-pyrrolo[3,2-c]pyridin-6-yl)amino)-N-(methyl-d3)nicotinamide (18g)

To a solution of 18f (70 mg, 0.37 mmol) and 4,6-dichloro-N-(methyl-d₃)nicotinamide (152.3 mg, 0.73 mmol) in THF (7 mL) stirred at 0° C. was added LiHMDS (1.84 mL, 1 mol/L in THF) dropwise. The reaction mixture was stirred at 0° C. for 4 h. A solution of sat. NH₄Cl (2 mL) was added to the mixture. The reaction was extracted with DCM (15 mL*3). The combined organic phase was washed with brine, dried over Na₂SO₄ and concentrated. The residue was purified by prep-TLC (DCM/MeOH=10/1) to give 18g (100 mg, 75% yield) as a yellow solid. LC-MS (ESI, Method 2), t_{R}=1.36 min, m/z (M+H)⁺=363.0.

### Step 8. 6-(Cyclopropanecarboxamido)-4-((1-ethyl-7-methoxy-1H-pyrrolo[3,2-c]pyridin-6-yl)amino)-N-(methyl-d₃)nicotinamide (18)

To a mixture of 18g (100 mg, 0.28 mmol), cyclopropanecarboxamide (117.3 mg, 1.38 mmol) and Cs₂CO₃ (179.6 mg, 0.55 mmol) in dioxane (10 mL) stirred under nitrogen at 25° C. was added BrettPhos (29.6 mg, 0.055 mmol), BrettPhos Pd G3 (25.0 mg, 0.027 mmol). The reaction mixture was stirred at 90° C. for 16 h. Water (20 mL) was added to the mixture. The mixture was extracted with EA (30 mL*2). The combined organic phase was washed with brine, dried over Na₂SO₄ and concentrated. The residue was purified by prep-TLC (DCM/MeOH=10/1) to give 18 (30 mg, crude). The crude product was purified by prep-HPLC (Method E) to give 18 (9.5 mg, 8% yield) as a white solid. ¹H NMR (400 MHz, DMSO-d₆) δ 11.30 (s, 1H), 10.62 (s, 1H), 9.02 (s, 1H), 8.58 (s, 1H), 8.51 (s, 1H), 8.36 (s, 1H), 7.38 (d, J=3.2 Hz, 1H), 6.55 (d, J=3.2 Hz, 1H), 4.38-4.31 (q, J=7.2 Hz, 2H), 3.83 (s, 3H), 2.02-1.95 (m, 1H), 1.37 (t, J=7.2 Hz, 3H), 0.80-0.74 (m, 4H). LC-MS (ESI, Method 4), t_{R}=2.45 min, m/z (M+H)⁺=412.2.

### Example 19

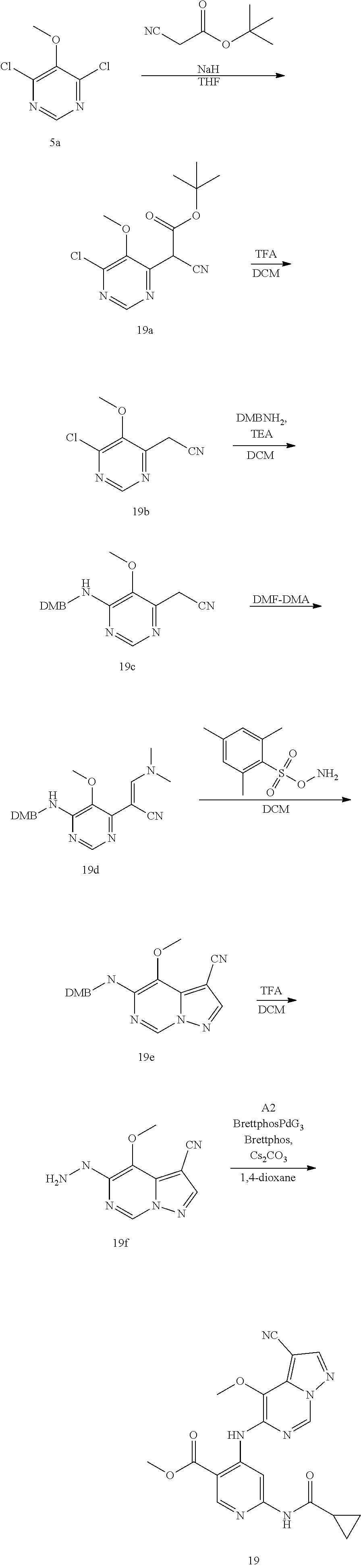

### Step 1. Tert-butyl 2-(6-chloro-5-methoxypyrimidin-4-yl)-2-cyanoacetate (19a)

To a solution of tert-butyl 2-cyanoacetate (9.46 g, 67.04 mmol) in THF (200 mL) was added NaH (5.36 g, 134.08 mmol, 60% in mineral oil) at 0° C. After stirring at 0° C. for 30 min, to it was added a solution of 5a (10 g, 55.86 mmol) in THF (20 mL). The mixture was stirred at 80° C. for 2 h. After cooling to r.t., the reaction mixture was poured into ice-water (150 mL) and acidified with 2 N HCl to pH=2. The mixture was extracted with EtOAc (300 mL*2). The combined organic layer was washed with brine (100 mL), dried over Na₂SO₄, filtered. The filtrate was concentrated under reduced pressure to afford 19a (15 g, 95% yield) as a yellow solid. LC-MS (ESI, Method 3) t_{R}=1.13 min, m/z (M+H-56)⁺=228.2.

### Step 2. 2-(6-Chloro-5-methoxypyrimidin-4-yl)acetonitrile (19b)

To a mixture of 19a (7.5 g, 26.44 mmol) in DCM (100 mL) was added TFA (50 mL) at 0° C. The mixture was stirred at r.t. for 4 h. The solvent was concentrated. The residue was diluted with H₂O (100 mL), adjusted to pH=9 with saturated Na₂CO₃ solution and extracted with EtOAc (100 mL*3). The combined organic layer was concentrated under reduced pressure to afford 19b (4.8 g, 99% yield) as a yellow oil. ¹H NMR (400 MHz, CDCl₃) δ 8.72 (s, 1H), 4.05 (s, 3H), 3.97 (s, 2H).

### Step 3. 2-(6-((2,4-Dimethoxybenzyl)amino)-5-methoxypyrimidin-4-yl)acetonitrile (19c)

A mixture of 19b (4.8 g, 26.14 mmol), TEA (7.3 mL, 52.3 mmol) and DMBNH₂ (5.68 g, 33.99 mmol) in EtOH (30 mL) was stirred at 80° C. for 2 h. After cooling to r.t., the mixture was diluted with H₂O (40 mL) and extracted with EtOAc (80 mL*2). The combined organic layer was concentrated under reduced pressure. The residue was purified by flash chromatography on silica gel (PE/EA=1/1) to afford 19c (5.35 g, 65% yield) as a yellow oil. LC-MS (ESI, Method 3) t_{R}=0.99 min, m/z (M+H)⁺=315.3.

### Step 4. (Z)-2-(6-((2,4-dimethoxybenzyl)amino)-5-methoxypyrimidin-4-yl)-3-(dimethylamino)acrylonitrile (19d)

A mixture of 19c (5.35 g, 17.02 mmol) in DMF-DMA (35 mL) was stirred at 130° C. for 10 h. The mixture was cooled and concentrated under reduced pressure to afford 19d (6.29 g, yield given) as a yellow oil. LC-MS (ESI, Method 3) t_{R}=1.06 min, m/z (M+H)⁺=370.2.

### Step 5. 5-((2,4-Dimethoxybenzyl)amino)-4-methoxypyrazolo[1,5-c]pyrimidine-3-carbonitrile (19e)

To a mixture of 19d (3.0 g, 8.12 mmol) in DCM (50 mL) was added dropwise a solution of O-(mesitylsulfonyl)hydroxylamine (2.10 g, 9.75 mmol) in DCM (10 mL) at 0° C. The mixture was stirred at 0° C. for 2 h. Another batch of O-(mesitylsulfonyl)hydroxylamine (0.58 g, 2.69 mmol) in DCM (5 mL) was added to the reaction mixture at 0° C. The mixture was stirred at r.t. for 18 h and purified by flash chromatography on silica gel (DCM/EA=20/1) to afford 19e (730 mg, 26% yield) as a yellow solid. ¹H NMR (400 MHz, CDCl₃) δ 8.80 (s, 1H), 8.03 (s, 1H), 7.21 (d, J=8.4 Hz, 1H), 6.47 (d, J=2.4 Hz, 1H), 6.41 (dd, J=8.4 Hz, 2.4 Hz, 1H), 5.56 (s, 1H), 4.64 (d, J=5.6 Hz, 2H), 3.90 (s, 3H), 3.89 (s, 3H), 3.76 (s, 3H).

### Step 6. 5-Amino-4-methoxypyrazolo[1,5-c]pyrimidine-3-carbonitrile (19f)

To a solution of 19e (400 mg, 1.18 mmol) in DCM (2 mL) was added TFA (2 mL) dropwise at r.t. The reaction mixture was stirred at r.t. for 1 h and concentrated under vacuum at 30° C. The residue was dissolved in H₂O (80 mL) and the solution was adjusted to pH=12 with 1 M NaOH solution. The mixture was extracted with DCM (80 mL*3). The combined organic layer was concentrated and the residue was purified by flash chromatography on silica gel (DCM/MeOH=10/1) to afford 19f (100 mg, 44% yield) as a yellow solid. LC-MS (ESI, Method 2) t_{R}=0.60 min, m/z (M+H)⁺=190.2.

### Step 7. Methyl 4-((3-cyano-4-methoxypyrazolo[1,5-c]pyrimidin-5-yl)amino)-6-(cyclopropanecarboxamido)nicotinate (19)

A mixture of 19f (100 mg, 0.53 mmol), A2 (148 mg, 0.58 mmol), BrettPhos Pd G3 (48 mg, 0.053 mmol), BrettPhos (28 mg, 0.053 mmol) and Cs₂CO₃ (344 mg, 1.06 mmol) in 1,4-dioxane (1 mL) was stirred at 90° C. overnight under N₂ atmosphere. After cooling to r.t., the mixture was concentrated. And the residue was purified by flash chromatography on silica gel (DCM/EA=20/1) to afford 19 (58 mg, 27% yield) as a brown solid. ¹H NMR (400 MHz, DMSO-d₆) δ 9.59 (s, 1H), 9.36 (s, 1H), 8.81 (s, 1H), 8.71 (s, 1H), 4.03 (s, 3H), 3.92 (s, 3H), 2.07-2.04 (m, 1H), 0.85-0.81 (m, 4H). LC-MS (ESI, Method 2) t_{R}=1.60 min, m/z (M+H)⁺=408.2.

### Example 20

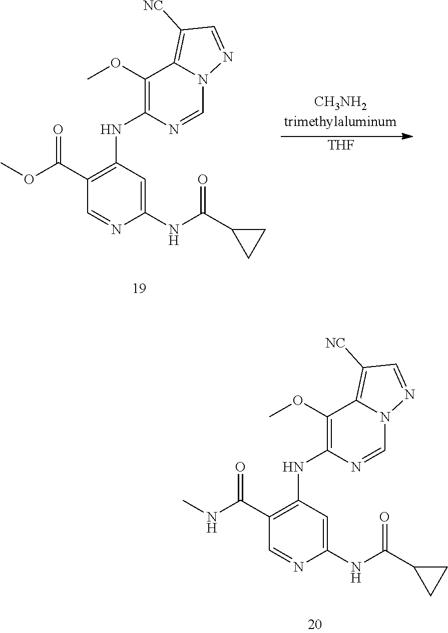 [SMILES: COC(=O)c1cnc(NC(=O)C2CC2)cc1Nc1ncn2ncc(C#N)c2c1OC ; CNC(=O)c1cnc(NC(=O)C2CC2)cc1Nc1ncn2ncc(C#N)c2c1OC ; "THF"]

### Step 1. 4-((3-Cyano-4-methoxypyrazolo[1,5-c]pyrimidin-5-yl)amino)-6-(cyclopropanecarboxamido)-N-methylnicotinamide (20)

To a solution of 19 (100 mg, 0.25 mmol) and CH₃NH₂ (2.45 mL, 2.45 mmol, 1 M in THF) in THF (4 mL) was slowly added trimethylaluminum (1.23 mL, 1.23 mmol, 1 M in petroleum ether) dropwise. The reaction was stirred at 70° C. for 5 h. After cooling to r.t., the reaction was quenched with H₂O (1 mL), adjusted to pH=6 with 2 M HCl and extracted with EtOAc (5 mL*2). The organic layer was separated, concentrated and the residue was purified by prep-HPLC (Method A) to afford 20 (6 mg, 6% yield) as a white solid. ¹H NMR (400 MHz, DMSO-d₆) δ 12.17 (s, 1H), 10.86 (s, 1H), 9.54 (s, 1H), 9.26 (s, 1H), 8.81 (s, 1H), 8.68 (s, 1H), 8.65 (s, 1H), 4.00 (s, 3H), 2.84 (d, J=4.4 Hz, 3H), 2.05-1.98 (m, 1H), 0.86-0.83 (m, 4H). LC-MS (ESI, Method 2) t_{R}=2.60 min, m/z (M+H)⁺=407.1.

### Example 21

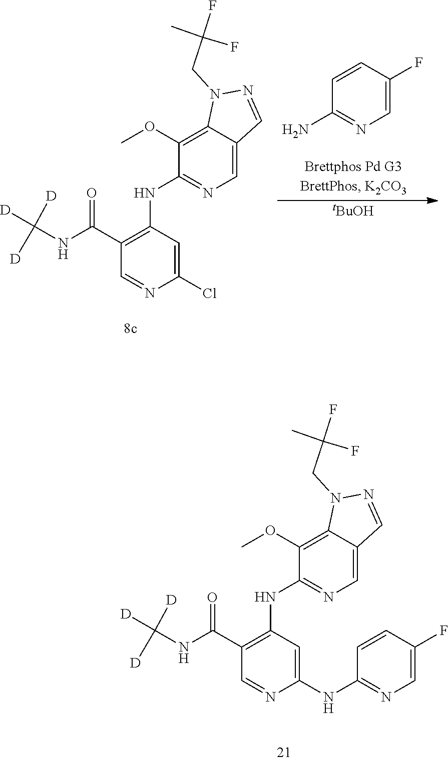

### Step 1. 4-((1-(2,2-Difluoropropyl)-7-methoxy-1H-pyrazolo[4,3-c]pyridin-6-yl)amino)-6-((5-fluoropyridin-2-yl)amino)-N-(methyl-d₃)nicotinamide (21)

To a solution of 8c (30 mg, 0.072 mmol), 5-fluoropyridin-2-amine (12 mg, 0.11 mmol) and K₂CO₃ (35 mg, 0.25 mmol) in BuOH (2 mL) was added BrettPhos Pd G3 (10 mg, 0.011 mmol) and BrettPhos (10 mg, 0.019 mmol) under N₂ atmosphere. The mixture was stirred at 110° C. for 5 h. The reaction mixture was diluted with water (20 mL), then extracted with EtOAc (20 mL*2). The combined organic layer was washed with brine (20 mL*2), dried over anhydrous Na₂SO₄, filtered and concentrated. The residue was purified by prep-HPLC (Method E) to give 21(12 mg, 34% yield) as a white solid. ¹H NMR (400 MHz, DMSO-d₆) δ 11.72 (s, 1H), 9.87 (s, 1H), 9.02 (s, 1H), 8.76 (s, 1H), 8.56 (s, 1H), 8.53 (s, 1H), 8.34 (s, 1H), 8.25 (d, J=2.8 Hz, 1H), 7.78-7.72 (m, 1H), 7.68-7.61 (m, 1H), 4.93 (t, J=13.2 Hz, 2H), 3.91 (s, 3H), 1.71 (t, J=19.2 Hz, 3H). LC-MS (ESI, Method 4) t_{R}=2.32 min, m/z (M+H)⁺=490.3.

### Example 22

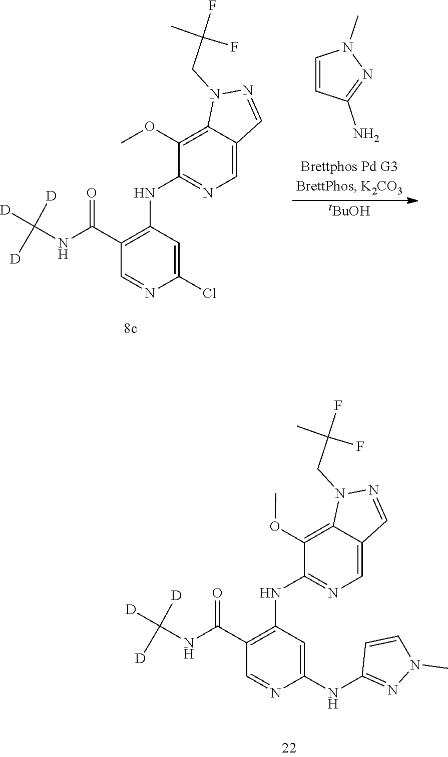

### Step 1. 4-((1-(2,2-Difluoropropyl)-7-methoxy-1H-pyrazolo[4,3-c]pyridin-6-yl)amino)-N-(methyl-d3)-6-((1-methyl-1H-pyrazol-3-yl)amino)nicotinamide (22)

To a solution of 8c (30 mg, 0.072 mmol), 1-methyl-1H-pyrazol-3-amine (11 mg, 0.11 mmol) and K₂CO₃ (35 mg, 0.25 mmol) in BuOH (2 mL) was added BrettPhos Pd G3 (10 mg, 0.011 mmol) and BrettPhos (10 mg, 0.019 mmol) under N₂ atmosphere. The mixture was stirred at 110° C. for 2 h. The reaction mixture was diluted with water (20 mL), then extracted with EtOAc (20 mL*2). The combined organic layer was washed with brine (20 mL*2), dried over anhydrous Na₂SO₄, filtered and concentrated. The residue was purified by prep-HPLC (Method E) to give 22 (21 mg, 60% yield) as a white solid. ¹H NMR (400 MHz, DMSO-d₆) δ 11.74 (s, 1H), 9.32 (s, 1H), 8.71 (s, 1H), 8.64 (s, 1H), 8.47-8.44 (m, 2H), 8.33 (s, 1H), 7.53 (d, J=2.4 Hz, 1H), 6.22 (d, J=2.4 Hz, 1H), 4.93 (t, J=13.2 Hz, 2H), 3.90 (s, 3H), 3.76 (s, 3H), 1.70 (t, J=19.2 Hz, 3H). LC-MS (ESI, Method 4) t_{R}=2.08 min, m/z (M+H)⁺=475.4.

### Example 23

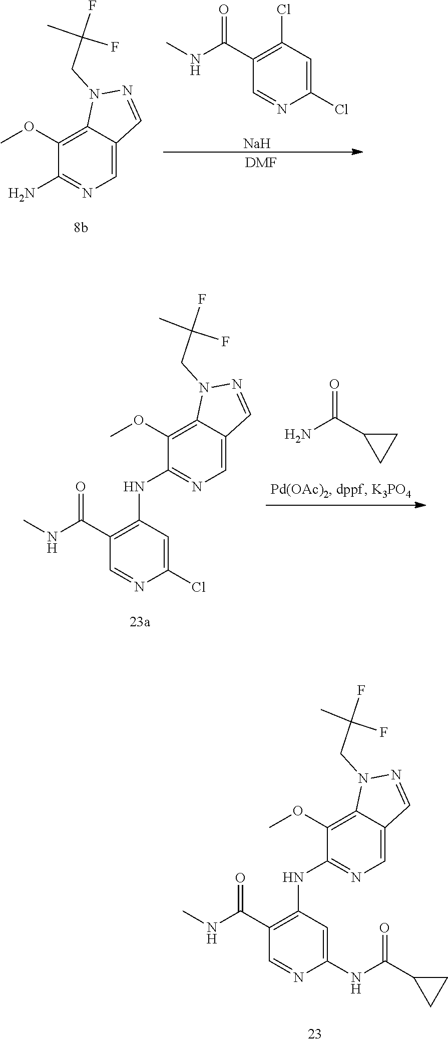

### Step 1. 6-Chloro-4-((1-(2,2-difluoropropyl)-7-methoxy-1H-pyrazolo[4,3-c]pyridin-6-yl)amino)-N-methylnicotinamide (23a)

To a solution of 8b (60 mg, 0.25 mmol) in DMF (4 mL) was added NaH (40 mg, 0.99 mmol, 60% in mineral oil) at 0° C. The mixture was stirred for 30 min, then 4,6-dichloro-N-methylnicotinamide (66 mg, 0.32 mmol) was added. The mixture was stirred at room temperature for 12 h. The mixture was quenched with sat. NH₄Cl (20 mL) and extracted with EtOAc (20 mL*2). The combined organic layer was washed with brine (15 mL*2), dried and condensed by vacuum. The residue was purified by flash chromatography (EtOAc in DCM is 65%) to give 23a (53 mg, 52% yield) as a yellow solid. LC-MS (ESI, Method 4) t_{R}=2.81 min, m/z (M+H)⁺=411.2.

### Step 2. 6-(Cyclopropanecarboxamido)-4-((1-(2,2-difluoropropyl)-7-methoxy-1H-pyrazolo[4,3-c]pyridin-6-yl)amino)-N-methylnicotinamide (23)

To a solution of 23a (20 mg, 0.049 mmol) and cyclopropanecarboxamide (17 mg, 0.19 mmol) in dioxane (2 mL) was added Pd(OAc)₂ (2 mg, 0.009 mmol), dppf (11 mg, 0.019 mmol) and K₃PO₄ (31 mg, 0.015 mmol) under N₂ atmosphere. The mixture was stirred at 100° C. for 3 h. The reaction mixture was diluted with water (20 mL), then extracted with EtOAc (20 mL*2). The combined organic layer was washed with brine (20 mL*2), dried over anhydrous Na₂SO₄, filtered and concentrated. The residue was purified by prep-HPLC (Method E) to give 23 (6 mg, 29% yield) as a white solid. ¹H NMR (400 MHz, DMSO-d₆) δ 11.64 (s, 1H), 10.73 (s, 1H), 9.22 (s, 1H), 8.76-8.64 (m, 2H), 8.56 (s, 1H), 8.32 (s, 1H), 4.93 (t, J=13.2 Hz, 2H), 3.89 (s, 3H), 2.82 (d, J=4.4 Hz, 3H), 2.05-1.97 (m, 1H), 1.70 (t, J=19.2 Hz, 3H), 0.84-0.76 (m, 4H). LC-MS (ESI, Method 4) t_{R}=1.97 min, m/z (M+H)⁺=460.3.

### Example 24

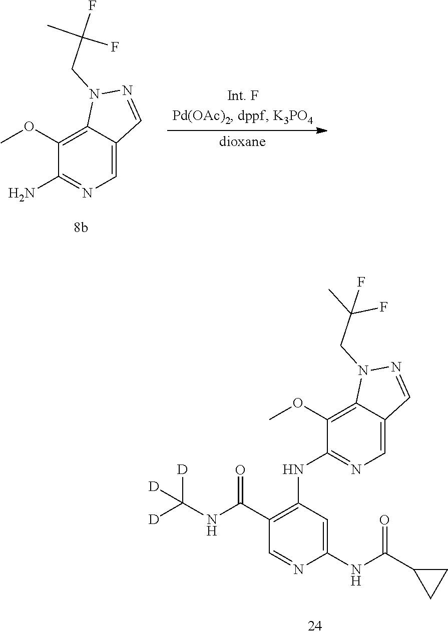 [SMILES: COc1c(N)ncc2cnn(CC(C)(F)F)c12 ; [2H]C([2H])([2H])NC(=O)c1cnc(NC(=O)C2CC2)cc1Nc1ncc2cnn(CC(C)(F)F)c2c1OC]

### Step 1. 6-(Cyclopropanecarboxamido)-4-((1-(2,2-difluoropropyl)-7-methoxy-1H-pyrazolo[4,3-c]pyridin-6-yl)amino)-N-(methyl-d₃)pyridazine-3-carboxamide (24)

To a solution of 8b (10 mg, 0.041 mmol), Int. F (16 mg, 0.062 mmol), dppf (9 mg, 0.017 mmol), K₃PO₄ (26 mg, 0.12 mmol) in dioxane (3 mL) was added Pd(OAc)₂ (2 mg, 0.008 mmol) at r.t. The reaction was stirred at 100° C. for 6 h under nitrogen protection. The reaction was added into H₂O (10 mL) and extracted by EtOAc (20 mL). The combined organic layer was washed by brine (10 mL*2), dried over Na₂SO₄, filtered and concentrated in vacuo. The crude was purified by flash chromatography and prep-HPLC (Method E) to afford 24 (1.5 mg, 8% yield) as a white solid. ¹H NMR (400 MHz, DMSO-d₆) δ 12.15 (s, 1H), 11.32 (s, 1H), 9.63 (s, 1H), 9.26 (s, 1H), 8.73 (s, 1H), 8.36 (s, 1H), 4.94 (t, J=12.8 Hz, 2H), 3.93 (s, 3H), 2.18-2.08 (m, 1H), 1.70 (t, J=19.2 Hz, 3H), 0.91-0.83 (m, 4H). LC-MS (ESI, Method 4) t_{R}=2.13 min, m/z (M+H)⁺=464.3.

### Example 25

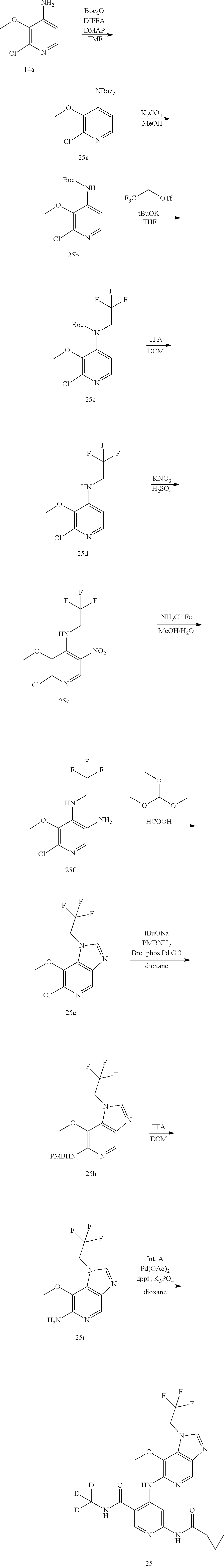

### Step 1. Tert-butyl N-[(tert-butoxy)carbonyl]-N-(2-chloro-3-methoxypyridin-4-yl)carbamate (25a)

To a solution of 14a (3 g, 18.92 mmol), DMAP (2.31 g, 18.92 mmol), DIPEA (4.89 g, 37.83 mmol, 6.59 mL) in THF (50 mL) was added Boc₂O (8.26 g, 8.68 mL) at an ice-bath, then the mixture was stirred at r.t. overnight. The mixture was diluted with H₂O (100 mL), extracted with EtOAc (100 mL*3), washed with brine (100 mL), dried over Na₂SO₄, filtered and the filtrate was concentrated to get the crude compound 25a (6.79 g, yield given) as a white solid. LC-MS (ESI, Method 4) t_{R}=3.44 min, m/z (M+H)⁺=359.3.

### Step 2. Tert-butyl (2-chloro-3-methoxypyridin-4-yl)carbamate (25b)

To a solution of 25a (6.79 g, 18.92 mmol) in MeOH (70 mL) was added K₂CO₃ (13.08 g, 94.62 mmol). Then the mixture was stirred at 70° C. for 4 h. The mixture was concentrated and diluted with H₂O (100 mL), extracted with EtOAc (50 mL*3), washed with brine (50 mL), dried over Na₂SO₄, filtered and the filtrate was concentrated and purified by flash chromatography (PE/EA=20/1 to 1/1) to get compound 25b (4.0 g, 82% yield) as a white solid. LC-MS (ESI, Method 4) t_{R}=2.97 min, m/z (M+H)⁺=259.1.

### Step 3. Tert-butyl (2-chloro-3-methoxypyridin-4-yl)(2,2,2-trifluoroethyl)carbamate (25c)

To a solution of 25b (500 mg, 1.93 mmol) in THF (5 mL) was added BuOK (281 mg, 2.51 mmol), then the mixture was stirred at 60° C. for 30 min. Then 2,2,2-trifluoroethyl trifluoromethanesulfonate (583 mg, 2.51 mmol) was added into the mixture and stirred at 60° C. for 2 h. Then the mixture was cooled to r.t. and another batch of ^{t}BuOK (281 mg, 2.51 mmol) was added. Then the mixture was stirred at 60° C. for 30 min. Then 2,2,2-trifluoroethyl trifluoromethanesulfonate (583 mg, 2.51 mmol) was added into the mixture and stirred at 60° C. for 2 h. The mixture was diluted with H₂O (30 mL), extracted with EtOAc (30 mL*3), washed with brine (30 mL), dried over Na₂SO₄, filtered and the filtrate was concentrated to get the crude compound 25c (500 mg, 76% yield) as a yellow solid. LC-MS (ESI, Method 4) t_{R}=3.37 min, m/z (M+H)⁺=341.2.

### Step 4. 2-Chloro-3-methoxy-N-(2,2,2-trifluoroethyl)pyridin-4-amine (25d)

To a solution of 25c (500 mg, 1.47 mmol) in DCM (5 mL) was added TFA (2.24 g, 19.60 mmol, 1.5 mL), then the mixture was stirred at r.t. for 4 h. The mixture was concentrated and diluted with H₂O (10 mL), adjusted to pH >7 with aq. Na₂CO₃, extracted with EtOAc (10 mL*3), washed with brine (20 mL), dried over Na₂SO₄, filtered and the filtrate was concentrated and purified by flash chromatography (PE/EA=20/1 to 2/1) to get compound 25d (233 mg, 66% yield) as a white solid. LC-MS (ESI, Method 4) t_{R}=2.07 min, m/z (M+H)⁺=241.0.

### Step 5. 2-Chloro-3-methoxy-5-nitro-N-(2,2,2-trifluoroethyl)pyridin-4-amine (25e)

To a solution of 25d (200 mg, 0.83 mmol) in conc. H₂SO₄ (2 mL) was added KNO₃ (109 mg, 1.08 mmol) at 0° C., then the mixture was stirred at r.t. for 6 h. The mixture was added into the ice-water dropwise, then basified to pH >9 with NH₃·H₂O, extracted with EtOAc (15 mL*4), washed with brine (20 mL), dried over Na₂SO₄ and concentrated to get compound 25e (160 mg, 67% yield) as a yellow solid. LC-MS (ESI, Method 4) t_{R}=2.93 min, m/z (M+H)⁺=286.0.

### Step 6. 6-Chloro-5-methoxy-N⁴-(2,2,2-trifluoroethyl)pyridine-3,4-diamine (25f)

A mixture of 25e (160 mg, 0.56 mmol), Fe powder (156 mg, 2.80 mmol), NH₄Cl (150 mg, 2.80 mmol) in MeOH (2 mL) and H₂O (0.5 mL) was stirred at 70° C. for 2 h. The mixture was filtered and the filter cake was washed with EtOAc (10 mL*2). The filtrate was concentrated and diluted with H₂O (10 mL), basified by NH₃·H₂O to pH >13, extracted with EtOAc (10 mL*3), washed with brine (10 mL), dried over Na₂SO₄, concentrated to get compound 25f (130 mg, 91% yield) as a yellow solid. LC-MS (ESI, Method 4) t_{R}=2.09 min, m/z (M+H)⁺=256.1.

### Step 7. 6-Chloro-7-methoxy-1-(2,2,2-trifluoroethyl)-1H-imidazo[4,5-c]pyridine (25g)

To a solution of 25f (130 mg, 0.51 mmol) in trimethoxymethane (2 mL) was added HCOOH (0.1 mL), then the mixture was stirred at 105° C. for 4 h. The mixture was concentrated, diluted with H₂O (20 mL), extracted with EtOAc (15 mL*3), washed with brine (15 mL), dried over Na₂SO₄ and concentrated to get the crude compound 25g (130 mg, 96% yield) as a yellow solid. LC-MS (ESI, Method 4) t_{R}=2.24 min, m/z (M+H)⁺=266.0.

### Step 8. 7-Methoxy-N-(4-methoxybenzyl)-1-(2,2,2-trifluoroethyl)-1H-imidazo[4,5-c]pyridin-6-amine (25h)

A mixture of 25g (120 mg, 0.45 mmol), (4-methoxyphenyl)methanamine (124 mg, 0.90 mmol), BrettPhos Pd G3 (82 mg, 0.09 mmol), tBuONa (108 mg, 1.13 mmol) in dioxane (1.5 mL) was stirred at 90° C. for 2 h. The mixture was concentrated and purified by flash chromatography (DCM/MeOH=100/1 to 20/1) to get compound 25h (150 mg, 90% yield) as a yellow solid. LC-MS (ESI, Method 4) t_{R}=2.24 min, m/z (M+H)⁺=367.2.

### Step 9. 7-Methoxy-1-(2,2,2-trifluoroethyl)-1H-imidazo[4,5-c]pyridin-6-amine (25i)

To a solution of 25h (150 mg, 0.41 mmol) in DCM (2 mL) was added TFA (2 mL), and the mixture was stirred at r.t. for 2 h. The mixture was concentrated and diluted with H₂O (20 mL), adjusted to pH >10 with aq. Na₂CO₃ and extracted with EtOAc (20 mL*3). The combined organic layer was washed with brine (30 mL*2), dried over Na₂SO₄, concentrated and purified by flash chromography (DCM/MeOH=100/1 to 5/1) to get compound 25i (57 mg, 57% yield) as a yellow solid. ¹H NMR (400 MHz, DMSO-d₆) δ 8.19 (s, 1H), 8.02 (s, 1H), 5.63 (s, 2H), 5.15 (q, J=9.2 Hz, 2H), 3.76 (s, 3H). LC-MS (ESI, Method 4) t_{R}=0.44 min, m/z (M+H)⁺=247.1.

### Step 10. 6-(Cyclopropanecarboxamido)-4-((7-methoxy-1-(2,2,2-trifluoroethyl)-1H-imidazo[4,5-c]pyridin-6-yl)amino)-N-(methyl-d₃)nicotinamide (25)

A mixture of 25i (10 mg, 0.041 mmol), Int. A (10 mg, 0.041 mmol), Pd(OAc)₂ (0.9 mg, 0.004 mmol), dppf (4.5 mg, 0.008 mmol), K₃PO₄ (17 mg, 0.08 mmol) in dioxane (0.5 mL) was stirred at 100° C. for 4 h. The mixture was concentrated and purified by prep-HPLC (Method E) to get compound 25 (6.3 mg, 33% yield) as a white solid. ¹H NMR (400 MHz, DMSO-d₆) δ 11.55 (s, 1H), 10.71 (s, 1H), 9.15 (s, 1H), 8.66 (s, 1H), 8.56 (s, 1H), 8.54 (s, 1H), 8.33 (s, 1H), 5.28 (q, J=9.2 Hz, 2H), 3.90 (s, 3H), 2.00-1.98 (m, 1H), 0.81-0.77 (m, 4H). LC-MS (ESI, Method 4) t_{R}=2.01 min, m/z (M+H)⁺=467.3.

### Example 26

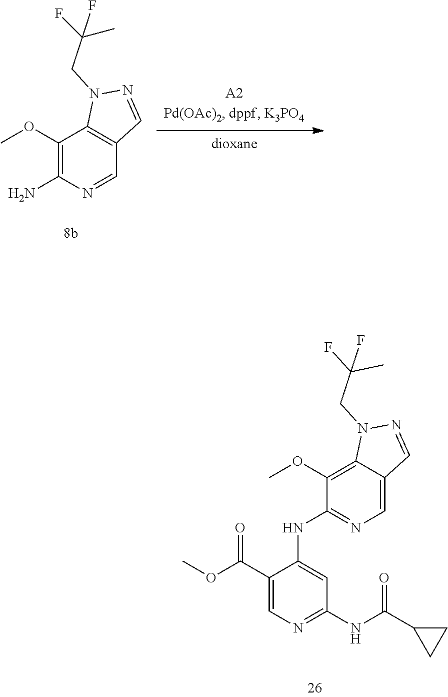 [SMILES: COc1c(N)ncc2cnn(CC(C)(F)F)c12 ; COC(=O)c1cnc(NC(=O)C2CC2)cc1Nc1ncc2cnn(CC(C)(F)F)c2c1OC]

### Step 1. Methyl 6-(cyclopropanecarboxamido)-4-((1-(2,2-difluoropropyl)-7-methoxy-1H-pyrazolo[4,3-c]pyridin-6-yl)amino)nicotinate (26)

To a solution of 8b (20 mg, 0.083 mmol), A2 (32 mg, 0.12 mmol), dppf (18 mg, 0.033 mmol), K₃PO₄ (53 mg, 0.25 mmol) in dioxane (3 mL) was added Pd(OAc)₂ (4 mg, 0.017 mmol) at r.t. The reaction was stirred at 100° C. for 6 h under nitrogen protection. The reaction was added into H₂O (10 mL) and extracted by EtOAc (20 mL). The combined organic layer was washed by brine (10 mL*2), dried over Na₂SO₄, filtered and concentrated in vacuo. The crude was purified by flash chromography and prep-HPLC (Method E) to afford 26 (25 mg, 66% yield) as a white solid. ¹H NMR (400 MHz, DMSO-d₆) δ 10.92 (s, 1H), 10.91 (s, 1H), 9.32 (s, 1H), 8.78 (s, 1H), 8.73 (s, 1H), 8.37 (s, 1H), 4.94 (t, J=13.2 Hz, 2H), 3.92 (s, 3H), 3.91 (s, 3H), 2.07-1.98 (m, 1H), 1.70 (t, J=19.2 Hz, 3H), 0.84-0.80 (m, 4H). LC-MS (ESI, Method 4) t_{R}=2.57 min, m/z (M+H)⁺=461.3.

### Example 27

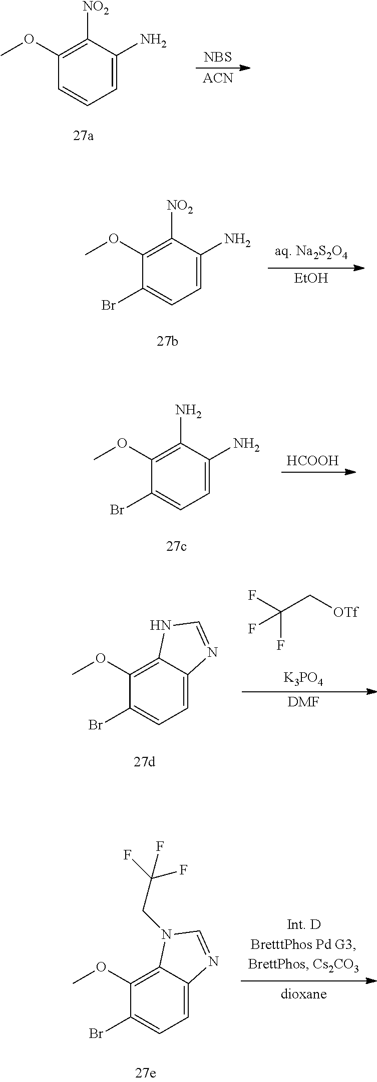

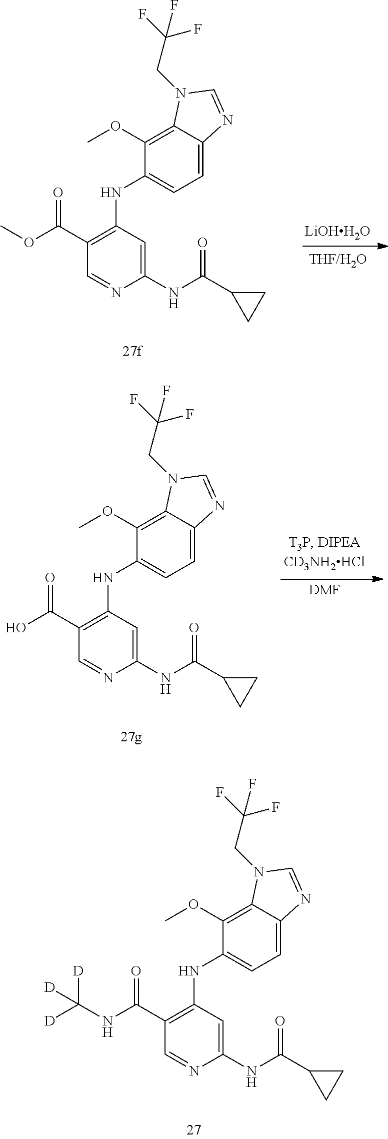

### Step 1. 3-Bromo-2-methoxy-6-nitro-aniline (27b)

To a solution of 27a (3.00 g, 17.84 mmol) in ACN (30 mL) was added NBS (3.22g, 17.84 mmol), then the mixture was stirred at r.t. for 2 h. The mixture was diluted with H₂ (100 mL), extracted with EtOAc (50 mL*3), washed with brine (50 mL), dried over anhydrous Na₂SO₄, concentrated and purified by flash chromatography (EA in PE is 5-20%) to give 27b (1.35 g, 31% yield) as a yellow solid. ¹H NMR (400 MHz, CDCl₃) δ 7.38 (d, J=8.8 Hz, 1H), 6.48 (d, J=9.2 Hz, 1H), 4.96 (brs, 2H), 3.99 (s, 3H). LC-MS (ESI, Method 4) t_{R}=2.76 min, m/z (⁷⁹Br, M+H)⁺=247.0.

### Step 2. 4-Bromo-3-methoxy-benzene-1,2-diamine (27c)

To a solution of 27b (500 mg, 2.02 mmol) in EtOH (8 mL) was added aq. Na₂S₂O₄ (8 mL, 1 M), then the mixture was stirred at 80° C. for 2 h. The mixture was diluted with H₂O (30 mL), extracted with EtOAc (30 mL*3), washed with brine (50 mL), dried over anhydrous Na₂SO₄ and concentrated to get the crude product 27c (400 mg, 91% yield) as a yellow solid. LC-MS (ESI, Method 4) t_{R}=1.21 min, m/z (⁷⁹Br, M+H)⁺=217.0.

### Step 3. 6-Bromo-7-methoxy-1H-benzimidazole (27d)

A solution of 27c (400 mg, 1.84 mmol) in HCOOH (5 mL) was stirred at 100° C. for 1 h. The mixture was concentrated and diluted with aq. NaHCO₃ (30 mL), extracted with EtOAc (30 mL*3), washed with brine (30 mL), dried over anhydrous Na₂SO₄, filtered and concentrated. The residue was purified by flash chromatography (MeOH in DCM is 0-10%) to give 27d (370 mg, 88% yield) as a yellow solid. LC-MS (ESI, Method 4) t_{R}=0.99 min, m/z (⁷⁹Br, M+H)⁺=227.0.

### Step 4. 6-Bromo-7-methoxy-1-(2,2,2-trifluoroethyl)benzimidazole (27e)

To a solution of 27d (100 mg, 0.44 mmol) in DMF (1.5 mL) was added K₃PO₄ (140 mg, 0.66 mmol) and 2,2,2-trifluoroethyl trifluoromethanesulfonate (112 mg, 0.48 mmol), then the mixture was stirred at 80° C. for 12 h. The mixture was diluted with H₂O (30 mL), extracted with EtOAc (20 mL*3), washed with brine (40 mL), dried over anhydrous Na₂SO₄, concentrated and purified by flash chromatography (PE in EA is 10-50%) to give 27e (40 mg, 29% yield) as a yellow gum. ¹H NMR (400 MHz, CDCl₃) δ 7.87 (s, 1H), 7.48-7.40 (m, 2H), 5.02 (q, J=8.4 Hz, 2H), 4.04 (s, 3H). LC-MS (ESI, Method 4) t_{R}=2.77 min, m/z (⁷⁹Br, M+H)⁺=309.0.

### Step 5. Methyl 6-(cyclopropanecarboxamido)-4-((7-methoxy-1-(2,2,2-trifluoroethyl)-1H-benzo[d]imidazol-6-yl)amino)nicotinate (27f)

A mixture of 27e (23 mg, 0.10 mmol), Int. D (35 mg, 0.10 mmol), BrettPhos Pd G3 (9 mg, 0.01 mmol), BrettPhos (11 mg, 0.02 mmol), Cs₂CO₃ (159 mg, 0.50 mmol) in dioxane (2 mL) was stirred at 100° C. for 16 h under N₂. The mixture was diluted with H₂O (10 mL), extracted with EtOAc (10 mL*3), washed with brine (20 mL), dried over anhydrous Na₂SO₄, concentrated and purified by flash chromatography (MeOH/DCM=10/1) to give 27f (25 mg, 55% yield) as a yellow solid. LC-MS (ESI, Method 4) t_{R}=2.98 min, m/z (M+H)⁺=464.2.

### Step 6. 6-(Cyclopropanecarboxamido)-4-((7-methoxy-1-(2,2,2-trifluoroethyl)-1H-benzo[d]imidazol-6-yl)amino)nicotinic acid (27g)

A mixture of 27f (25 mg, 0.05 mmol) and LiOH·H₂O (7 mg, 0.15 mmol) in co-solvent of THF (2 mL) and water (1 mL) was stirred at 20° C. for 24 h. A yellow solution was formed. The reaction mixture was concentrated and dried in vacuo to give 27g (24 mg, crude) as a yellow solid, which was used for the next step directly without further purification. LC-MS (ESI, Method 4) t_{R}=1.83 min, m/z (M+H)⁺=450.2.

### Step 7. 6-(Cyclopropanecarboxamido)-4-((7-methoxy-1-(2,2,2-trifluoroethyl)-1H-benzo[d]imidazol-6-yl)amino)-N-(methyl-d₃)nicotinamide (27)

A mixture of CD₃NH₂·HCl (6 mg, 0.08 mmol), 27g (24 mg, crude), DIPEA (21 mg, 0.16 mmol) and T₃P (52 mg, 0.08 mmol, 50% purity in EtOAc) in DMF (1 mL) was stirred at 30° C. for 3 days. A yellow solution was formed. The reaction mixture was diluted with water (30 mL) and extracted with EtOAc (40 mL*3). The combined organic layer was washed with water (40 mL*3) and brine (40 mL*2), dried over anhydrous Na₂SO₄, filtered and concentrated. The residue was purified by flash chromatography (MeOH in DCM is 0-10%) to give 27 (5.3 mg, 21% yield) as a white solid. ¹H NMR (400 MHz, DMSO-d₆) δ 10.68 (s, 1H), 10.41 (s, 1H), 8.60 (s, 1H), 8.51 (s, 1H), 8.26 (s, 1H), 7.70 (s, 1H), 7.47 (d, J=7.2 Hz, 1H), 7.19 (d, J=7.2 Hz, 1H), 5.30 (q, J=9.2 Hz, 2H), 3.78 (s, 3H), 1.97-1.90 (m, 1H), 0.76-0.66 (m, 4H). LC-MS (ESI, Method 4) t_{R}=3.33 min, m/z (M+H)⁺=466.3.

### Example 28

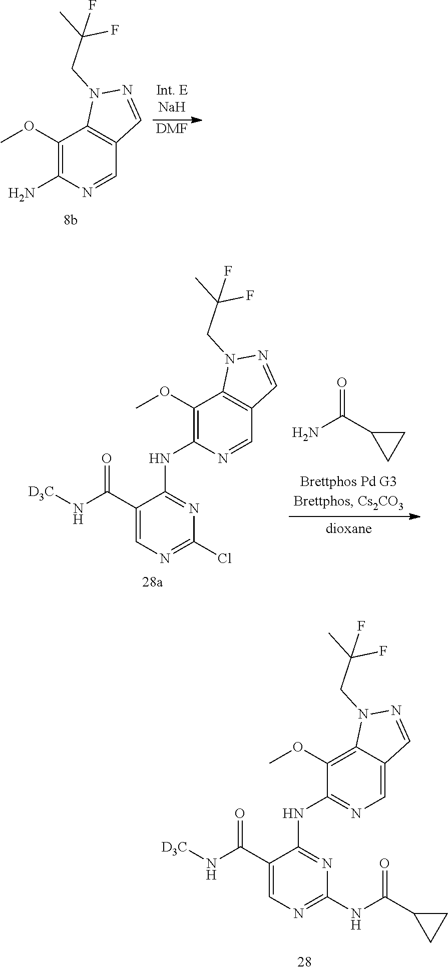

### Step 1. 2-Chloro-4-((1-(2,2-difluoropropyl)-7-methoxy-1H-pyrazolo[4,3-c]pyridin-6-yl)amino)-N-(methyl-d₃)pyrimidine-5-carboxamide (28a)

To a solution of 8b (70 mg, 0.29 mmol) and Int. E (91 mg, 0.43 mmol) in DMF (10 mL) was added NaH (35 mg, 1.44 mmol, 60% in mineral oil) at 0° C. under nitrogen protection. The reaction was stirred at 0° C. to r.t. for 3 h. H₂O (10 mL) was added and the solution was extracted by EtOAc (20 mL*2). The combined organic layer was washed by brine (10 mL*2), dried over Na₂SO₄, filtered and concentrated in vacuo. The crude was purified by flash chromatography (MeOH in DCM is 1-15%) to afford 28a (25 mg, 21% yield) as a yellow solid. LC-MS (ESI, Method 4) t_{R}=2.12 min, m/z (M+H)⁺=415.2.

### Step 2. 2-(Cyclopropanecarboxamido)-4-((1-(2,2-difluoropropyl)-7-methoxy-1H-pyrazolo[4,3-c]pyridin-6-yl)amino)-N-(methyl-d₃)pyrimidine-5-carboxamide (28)

To a solution of 28a (20 mg, 0.048 mmol), BrettPhos Pd G3 (9 mg, 0.01 mmol), BrettPhos (10 mg, 0.02 mmol), Cs₂CO₃ (47 mg, 0.14 mmol) in dioxane (2 mL) was added cyclopropane carboxamide (21 mg, 0.24 mmol) at r.t. The reaction was stirred at 100° C. for 3 h under nitrogen protection. H₂O (10 mL) was added and the solution was extracted by EtOAc (20 mL). The combined organic layer was washed by brine (10 mL*2), dried over Na₂SO₄, filtered and concentrated in vacuo. The crude was purified by prep-HPLC (Method E) to afford 28 (2.5 mg, 11% yield) as a light-yellow solid. ¹H NMR (400 MHz, DMSO-d₆) δ 11.12 (s, 1H), 10.28 (s, 1H), 8.74 (s, 1H), 8.72 (s, 1H), 8.67 (s, 1H), 8.37 (s, 1H), 4.98 (t, J=13.2 Hz, 2H), 3.83 (s, 3H), 2.73-2.61 (m, 1H), 1.70 (t, J=19.2 Hz, 3H), 0.72-0.64 (m, 2H), 0.49-0.41 (m, 2H). LC-MS (ESI, Method 4) t_{R}=2.12 min, m/z (M+H)⁺=464.3.

### Example 29

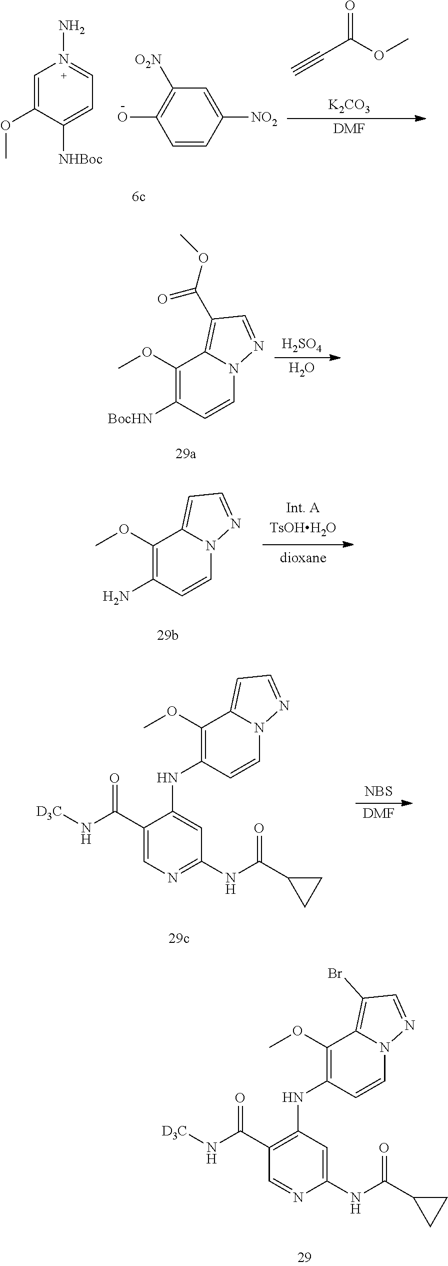

### Step 1. Methyl 5-((tert-butoxycarbonyl)amino)-4-methoxypyrazolo[1,5-a]pyridine-3-carboxylate (29a)

A mixture of methyl propiolate (749 mg, 8.91 mmol, 7.93 mL), 6c (1.89 g, 4.45 mmol) and K₂CO₃ (1.23 g, 8.91 mmol) in DMF (5 mL) was stirred at 20° C. for 2 h. A black suspension was formed. The reaction mixture was concentrated and diluted with water (50 mL), then extracted with EtOAc (50 mL*2). The combined organic layer was washed with water (50 mL*2), brine (50 mL*2), dried over anhydrous Na₂SO₄, filtered and concentrated. The residue was purified by flash chromatography (EtOAc in PE is 10-30%) to give 29a (350 mg, 24% yield) as a yellow solid. ¹H NMR (400 MHz, CDCl₃) δ 8.35 (s, 1H), 8.28 (d, J=7.6 Hz, 1H), 8.01 (d, J=7.6 Hz, 1H), 7.32 (brs, 1H), 3.89 (s, 3H), 3.88 (s, 3H), 1.55 (s, 9H). LC-MS (ESI, Method 4) t_{R}=4.13 min, m/z (M+H)⁺=322.1.

### Step 2. 4-Methoxypyrazolo[1,5-a]pyridin-5-amine (29b)

A mixture of 29a (350 mg, 1.09 mmol) in conc. H₂SO₄/H₂O (3 mL, v/v=1/1) was stirred at 60° C. for 2 h. After cooling to r.t., the mixture was basified with 10% aq. NaOH to pH=9 and extracted with EtOAc (10 mL*3). The organic layer was dried over Na₂SO₄, filtered and concentrated to afford 29b (152 mg, 86% yield) as a black solid. LC-MS (ESI, Method 3) t_{R}=0.79 min, m/z (M+H)⁺=164.0.

### Step 3. 6-(Cyclopropanecarboxamido)-4-((4-methoxypyrazolo[1,5-a]pyridin-5-yl)amino)-N-(methyl-d3)nicotinamide (29c)

A solution of 29b (150 mg, 0.92 mmol), Int. A (236 mg, 0.92 mmol) and TsOH·H₂O (4 mg, 0.02 mmol) in dioxane (0.5 mL) was stirred at 100° C. for 12 h. After cooling to r.t., the reaction mixture was concentrated and the residue was purified by flash chromatography on silica gel (DCM/MeOH=20/1) to afford compound 29c (90 mg, 26% yield) as a white solid. ¹H NMR (400 MHz, DMSO-d₆) δ 10.76 (s, 1H), 10.51 (s, 1H), 8.60 (s, 1H), 8.52 (s, 1H), 8.51 (d, J=7.2 Hz, 1H), 7.98 (d, J=2.4 Hz, 1H), 7.82 (s, 1H), 6.90 (d, J=7.2 Hz, 1H), 6.70 (d, J=2.4 Hz, 1H), 3.92 (s, 3H), 1.98-1.95 (m, 1H), 0.76-0.74 (m, 4H). LC-MS (ESI, Method 2) t_{R}=2.21 min, m/z (M+H)⁺=384.1.

### Step 4. 4-((3-Bromo-4-methoxypyrazolo[1,5-a]pyridin-5-yl)amino)-6-(cyclopropanecarboxamido)-N-(methyl-d3)nicotinamide (29)

To a solution of 29c (90 mg, 0.23 mmol) in DMF (1 mL) was added NBS (50 mg, 0.28 mmol) at 0° C. After stirring at 30° C. for 12 h, the reaction mixture was poured into H₂O (5 mL) and the formed solid was filtered. The filter cake was collected by filtration and purified by prep-HPLC (Method A) to afford 29 (12 mg, 11% yield) as a yellow solid. ¹H NMR (400 MHz, DMSO-d₆) δ 10.83 (s, 1H), 10.74 (s, 1H), 8.65 (s, 1H), 8.59 (d, J=7.6 Hz, 1H), 8.56 (s, 1H), 8.07 (s, 1H), 7.91 (s, 1H), 7.02 (d, J=7.6 Hz, 1H), 3.82 (s, 3H), 1.99-1.96 (m, 1H), 0.78-0.75 (m, 4H). LC-MS (ESI, Method 2) t_{R}=1.11 min, m/z (⁷⁹Br, M+H)⁺=462.0.

### Example 30

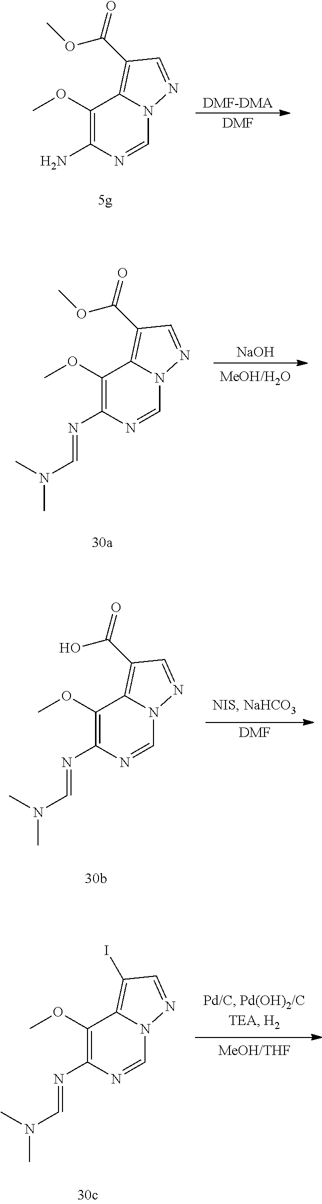

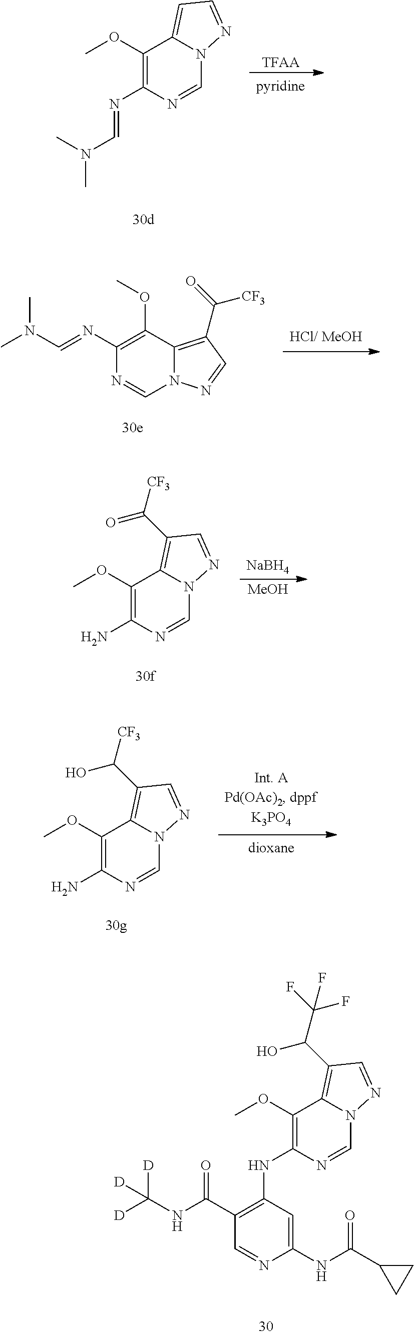

### Step 1. Methyl 5-amino-4-methoxypyrazolo[1,5-c]pyrimidine-3-carboxylate (30a)

A mixture of 5g (6.7 g, 30.15 mmol), DMF-DMA (7.19 g, 60.31 mmol) in DMF (20 mL) was stirred at 50° C. for 4 h. The mixture was concentrated to afford 30a (8 g, 96% yield) as a brown oil, which was used for the next step directly without further purification. LC-MS (ESI, Method 3) t_{R}=0.73 min, m/z (M+H)⁺=278.3.

### Step 2. 5-(((Dimethylamino)methylene)amino)-4-methoxypyrazolo[1,5-c]pyrimidine-3-carboxylic acid (30b)

To a mixture of 30a (7.5 g, 27.05 mmol) in MeOH (50 mL) and H₂O (25 mL) was added NaOH (2.16 g, 54.10 mmol). The mixture was stirred at 45° C. for 4 h and diluted with 25 mL of water. The resultant mixture was concentrated to remove organic layer and the aqueous layer was adjusted to pH=1 with 2 M HCl. The formed solid was filtered and dried to afford 30b (7.12 g, yield given) as a white solid. ¹H NMR (400 MHz, DMSO-d₆) δ 9.57 (s, 1H), 8.95 (s, 1H), 8.58 (s, 1H), 3.86 (s, 3H), 3.45 (s, 3H), 3.38 (s, 3H).

### Step 3. N′-(3-iodo-4-methoxypyrazolo[1,5-c]pyrimidin-5-yl)-N,N-dimethylformimidamide (30c)

To a solution of 30b (4 g, 11.40 mmol) in DMF (30 mL) was added NaHCO₃ (1.91 g, 22.79 mmol) at 0° C. To the reaction mixture was added NIS (5.13 g, 22.79 mmol) portionwise over 10 min. The reaction was stirred at 15° C. for 1 h and diluted with EtOAc (80 mL). The mixture was washed with sat. Na₂S₂O₃ (30 mL*2), sat. NaHCO₃ (30 mL*2) and brine (50 mL*2), dried over Na₂SO₄, filtered and concentrated. The residue was stirred in acetonitrile (5 mL) for 30 min and filtered. The filter cake was dried to afford 30c (1.63 g, 41% yield) as a white solid. LC-MS (ESI, Method 3) t_{R}=0.88 min, m/z (M+H)⁺=346.1.

### Step 4. N′-(4-methoxypyrazolo[1,5-c]pyrimidin-5-yl)-N,N-dimethylformimidamide (30d)

To a solution of 30c (600 mg, 1.74 mmol) and TEA (352 mg, 3.48 mmol, 0.48 mL) in MeOH/THF (4 mL/4 mL) was added Pd/C (60 mg, 10% on carbon wetted with 50% water) and Pd(OH)₂/C (60 mg, 10% on carbon wetted with 55% water) at 10° C. The mixture was stirred at 30° C. under H₂ (1 atm) for 24 h. The mixture was filtered and the filtrate was concentrated to afford 30d (350 mg, 92% yield) as a yellow solid, which was used for the next step directly without further purification. LC-MS (ESI, Method 3) t_{R}=0.66 min, m/z (M+H)⁺=220.1.

### Step 5. N′-(4-methoxy-3-(2,2,2-trifluoroacetyl)pyrazolo[1,5-c]pyrimidin-5-yl)-N,N-dimethylformimidamide (30e)

To a solution of 30d (350 mg, 1.60 mmol) in pyridine (3 mL) was slowly added trifluoroacetic anhydride (503 mg, 2.39 mmol, 0.33 mL) at 0° C. The reaction was stirred at 0° C. for 10 min and at 30° C. for 12 h. The reaction mixture was concentrated to dryness and purified by flash chromatography on silica gel (PE/EA=1/1) to afford 30e (150 mg, 30% yield) as a brown solid. LC-MS (ESI, Method 3) t_{R}=0.97 min, m/z (M+H)⁺=316.3.

### Step 6. 1-(5-Amino-4-methoxypyrazolo[1,5-c]pyrimidin-3-yl)-2,2,2-trifluoroethan-1-one (30f)

To a solution of 30e (150 mg, 0.48 mmol) in HCl/MeOH (0.5 M, 0.5 mL) was stirred at 80° C. for 1.5 h. The solution was basified with sat. Na₂CO₃ (3 mL) to pH >9 and extracted with EtOAc (3 mL*3). The organic layer was concentrated and purified by prep-TLC (DCM/MeOH=10/1) to get compound 30f (35 mg, 28% yield) as a yellow solid. LC-MS (ESI, Method 3) t_{R}=1.04 min, m/z (M+H)⁺=261.0.

### Step 7. 1-(5-Amino-4-methoxypyrazolo[1,5-c]pyrimidin-3-yl)-2,2,2-trifluoroethan-1-ol (30g)

To a solution of 30f (35 mg, 0.13 mmol) in MeOH (1 mL) was added NaBH₄ (11 mg, 0.27 mmol) at −5° C. After stirred at −5° C. for 0.5 h, the mixture was diluted with water (1 mL) and extracted with EtOAc (3 mL). The organic layer was washed with brine (1 mL), dried over Na₂SO₄, and filtered. The filtrate was concentrated to afford 30g (30 mg, 85% yield) as a yellow solid. ¹H NMR (400 MHz, CDCl₃) δ 8.80 (s, 1H), 7.98 (s, 1H), 5.39-5.37 (m, 1H), 4.49 (s, 2H), 3.91 (s, 3H), 3.43-3.41 (m, 1H). LC-MS (ESI, Method 3) t_{R}=0.92 min, m/z (M+H)⁺=263.0.

### Step 8. 6-(Cyclopropanecarboxamido)-4-((4-methoxy-3-(2,2,2-trifluoro-1-hydroxyethyl)pyrazolo[1,5-c]pyrimidin-5-yl)amino)-N-(methyl-d₃)nicotinamide (30)

A mixture of 30g (10 mg, 0.038 mmol), Int. A (10 mg, 0.038 mmol), dppf (8 mg, 0.015 mmol), Pd(OAc)₂ (2 mg, 0.0076 mmol), K₃PO₄ (24 mg, 0.114 mmol) in dioxane (0.5 mL) was stirred at 100° C. for 16 h. The mixture was concentrated and purified by prep-HPLC (Method E) to get compound 30 (10 mg, 54% yield) as a white solid. ¹H NMR (400 MHz, DMSO-d₆) δ 11.66 (s, 1H), 10.80 (s, 1H), 9.37 (s, 1H), 8.96 (s, 1H), 8.72 (s, 1H), 8.59 (s, 1H), 8.20 (s, 1H), 6.89 (d, J=6.0 Hz, 1H), 5.50-5.43 (m, 1H), 3.89 (s, 3H), 2.02-1.97 (m, 1H), 0.81-0.79 (m, 4H). LC-MS (ESI, Method 4) t_{R}=1.98 min, m/z (M+H)⁺=483.3.

### Example 31

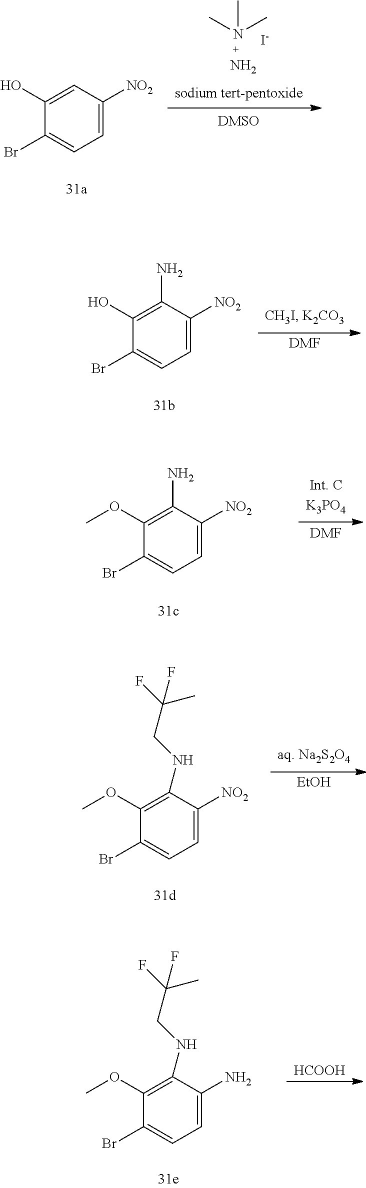

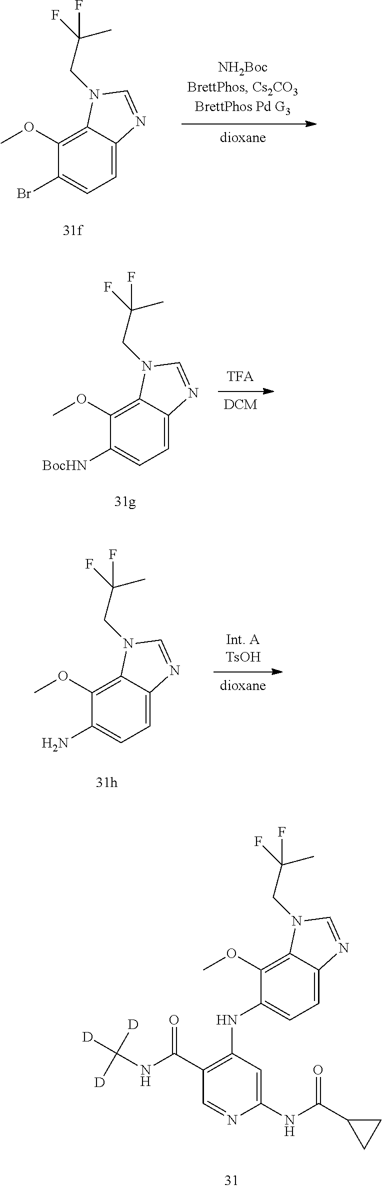 [SMILES: COc1c(Br)ccc2ncn(CC(C)(F)F)c12 ; COc1c(NC(=O)OC(C)(C)C)ccc2ncn(CC(C)(F)F)c12 ; COc1c(N)ccc2ncn(CC(C)(F)F)c12 ; [2H]C([2H])([2H])NC(=O)c1cnc(NC(=O)C2CC2)cc1Nc1ccc2ncn(CC(C)(F)F)c2c1OC]

### Step 1. 2-Amino-6-bromo-3-nitro-phenol (31b)

To a solution of 31a (4.50 g, 20.64 mmol) in DMSO (60 mL) was added 1,1,1-trimethylhydrazinium iodide (4.59 g, 22.71 mmol). The mixture was stirred at r.t. for 90 min, then sodium tert-pentoxide (11.37 g, 103.21 mmol) was added. The resulting mixture was further stirred at r.t. for 2 h. Aq. HCl (62 mL, 2 M) was added at 0° C. and the mixture was stirred for 1 h. The mixture was diluted with H₂O (100 mL), extracted with EtOAc (50 mL*3), washed with brine (100 mL), dried over Na₂SO₄ and concentrated to get compound 31b (4.76 g, 98% yield) as a yellow solid. ¹H NMR (400 MHz, DMSO-d₆) δ 10.00 (brs, 1H), 7.49 (d, J=9.6 Hz, 1H), 7.18 (brs, 2H), 6.76 (d, J=9.6 Hz, 1H). LC-MS (ESI, Method 4) t_{R}=2.29 min, m/z (⁷⁹Br, M+H)⁺=233.0.

### Step 2. 3-Bromo-2-methoxy-6-nitro-aniline (31c)

A mixture of 31b (4.76 g, 20.43 mmol), K₂CO₃ (6.21 g, 44.94 mmol) and iodomethane (3.19 g, 22.47 mmol, 1.40 mL) in DMF (50 mL) was stirred at 20° C. for 2 h. A brown suspension was formed. The reaction mixture was diluted with water (200 mL) and extracted with EtOAc (100 mL*3). The combined organic layer was washed with water (100 mL*3) and brine (100 mL*2), dried over anhydrous Na₂SO₄, filtered and concentrated. The residue was purified by flash chromatography (EtOAc in PE is 10-60%) to give 31c (3.10 g, 61% yield) as a yellow solid. LC-MS (ESI, Method 4) t_{R}=2.86 min, m/z (⁷⁹Br, M+H)⁺=247.0.

### Step 3. 3-Bromo-N-(2,2-difluoropropyl)-2-methoxy-6-nitro-aniline (31d)

To a solution of 31c (300 mg, 1.21 mmol) in DMF (5 mL) was added K₃PO₄ (516 mg, 2.43 mmol) and Int. C (489 mg, 1.82 mmol). The resulting mixture was stirred at 30° C. for 48 h. The mixture was diluted with H₂O (50 mL), extracted with EtOAc (50 mL*3), washed with water (50 mL*3) and brine (50 mL*2), dried over anhydrous Na₂SO₄, filtered and concentrated. The residue was purified by flash chromatography (EtOAc in PE is 5-20%) to give 31d (200 mg, 51% yield) as a yellow oil. LC-MS (ESI, Method 4) t_{R}=3.40 min, m/z (⁷⁹Br, M+H)⁺=325.0.

### Step 4. 5-Bromo-N¹-(2,2-difluoropropyl)-6-methoxybenzene-1,2-diamine (31e)

To a solution of 31d (200 mg, 0.61 mmol) in EtOH (5 mL) was added aq. Na₂S₂O₄ (5.0 mL, 1 M). The resulting mixture was stirred at 80° C. for 2 h. The reaction mixture was concentrated and diluted with water (50 mL), then extracted with EtOAc (50 mL*2). The combined organic layer was washed with water (50 mL*2) and brine (50 mL*2), dried over anhydrous Na₂SO₄, filtered and concentrated to give 31e (180 mg, crude) as a yellow oil, which was used for the next step directly without further purification. LC-MS (ESI, Method 4) t_{R}=2.96 min, m/z (⁷⁹Br, M+H)⁺=295.1.

### Step 5. 6-Bromo-1-(2,2-difluoropropyl)-7-methoxy-benzimidazole (31f)

A mixture of 31e (180 mg, 0.61 mmol) in HCOOH (1 mL) was stirred at 100° C. for 2 h. The reaction mixture was concentrated in vacuo and diluted with aq. NaHCO₃ (50 mL), then extracted with EtOAc (50 mL*2). The combined organic layer was washed with water (50 mL*2) and brine (50 mL*2), dried over anhydrous Na₂SO₄, filtered and concentrated. The residue was purified by flash chromatography (MeOH in DCM is 0-10%) to give 31f (100 mg, 54% yield for 2 steps) as a light yellow solid. LC-MS (ESI, Method 4) t_{R}=2.64 min, m/z (⁷⁹Br, M+H)⁺=305.1.

### Step 6. Tert-butyl (1-(2,2-difluoropropyl)-7-methoxy-1H-benzo[d]imidazol-6-yl)carbamate (31g)

A mixture of 31f (90 mg, 0.29 mmol), tert-butyl carbamate (52 mg, 0.44 mmol), BrettPhos Pd G3 (27 mg, 0.03 mmol), BrettPhos (32 mg, 0.59 mmol), K₃PO₄ (313 mg, 1.47 mmol) in dioxane (3 mL) was stirred at 100° C. for 16 h under N₂. The reaction mixture was diluted with EtOAc (50 mL) and filtered. The filtrate was washed with water (50 mL) and brine (50 mL), dried over anhydrous Na₂SO₄, filtered and concentrated. The residue was purified by flash chromatography (MeOH in DCM is 0-8%) to give 31g (100 mg, 99% yield) as a yellow solid. LC-MS (ESI, Method 4) t_{R}=2.45 min, m/z (M+H)⁺=342.2.

### Step 7. 3-(2,2-Difluoropropyl)-4-methoxy-benzimidazol-5-amine (31h)

To a solution of 31g (100 mg, 0.29 mmol) in DCM (2 mL) was added TFA (1 mL). The mixture was stirred at r.t. for 1 h. The mixture was concentrated and quenched with sat. NaHCO₃ (40 mL), then extracted with EtOAc (20 mL*3). The organic layer was washed with aq. NaHCO₃ (30 mL) and brine (30 mL). The separated solution was dried over anhydrous Na₂SO₄, filtered and concentrated. The residue was purified by flash chromatography (MeOH in DCM is 0-10%) to give 31h (60 mg, 85% yield) as a yellow solid. LC-MS (ESI, Method 4) t_{R}=0.51 min, m/z (ESI, M+H)⁺=242.0.

### Step 8. 6-(Cyclopropanecarboxamido)-4-((1-(2,2-difluoropropyl)-7-methoxy-1H-benzo[d]imidazol-6-yl)amino)-N-(methyl-d₃)nicotinamide (31)

A mixture of 31h (60 mg, 0.25 mmol), Int. A (58 mg, 0.22 mmol), pTSA (58 mg, 0.34 mmol) in dioxane (3 mL) was stirred at 100° C. for 24 h. The reaction mixture was concentrated in vacuo and diluted with aq. NaHCO₃ (30 mL), then extracted with EtOAc (30 mL*2). The combined organic layer was washed with water (50 mL*2) and brine (50 mL*2), dried over anhydrous Na₂SO₄, filtered and concentrated. The residue was purified by flash chromatography (MeOH in DCM is 0-10%) to give a yellow solid, which was further purified by prep-HPLC (Method D) to get 31(30.0 mg, 26% yield) as a white solid. ¹H NMR (400 MHz, DMSO-d₆) δ 10.66 (s, 1H), 10.37 (s, 1H), 8.58 (s, 1H), 8.50 (s, 1H), 8.14 (s, 1H), 7.67 (s, 1H), 7.44 (d, J=8.4 Hz, 1H), 7.15 (d, J=8.4 Hz, 1H), 4.87 (t, J=14.4 Hz, 2H), 3.75 (s, 3H), 1.96-1.89 (m, 1H), 1.68 (t, J=19.2 Hz, 3H), 0.74-0.66 (m, 4H). LC-MS (ESI, Method 4) t_{R}=1.71 min, m/z (M+H)⁺=462.4.

### Example 32

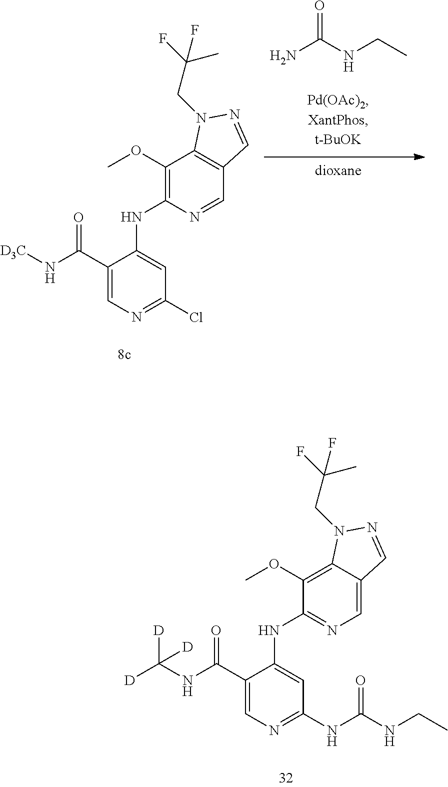

### Step 1. 4-((1-(2,2-Difluoropropyl)-7-methoxy-1H-pyrazolo[4,3-c]pyridin-6-yl)amino)-6-(3-ethylureido)-N-(methyl-d3)nicotinamide (32)

To a solution of 8c (10 mg, 0.024 mmol), Pd(OAc)₂ (1 mg, 0.005 mmol), XantPhos (6 mg, 0.01 mmol), t-BuOK (8 mg, 0.072 mmol) in dioxane (3 mL) was added 1-ethylurea (11 mg, 0.12 mmol) at r.t. The reaction was stirred at 100° C. for 5 h under nitrogen protection. The resulting solution was added into H₂O (10 mL) and extracted by EA (20 mL). The combined organic layer was washed by brine (10 mL*2), dried over Na₂SO₄ and filtered. The filtrate was concentrated in vacuo. The crude was purified by prep-HPLC (Method E) to afford 32 (4 mg, 35% yield) as a white solid. ¹H NMR (400 MHz, DMSO-d₆) δ 11.62 (s, 1H), 9.26 (s, 1H), 8.69 (s, 1H), 8.61 (s, 1H), 8.50 (s, 1H), 8.47 (s, 1H), 8.34 (s, 1H), 8.31-8.25 (m, 1H), 4.93 (t, J=13.2 Hz, 2H), 3.90 (s, 3H), 3.24-3.13 (m, 2H), 1.70 (t, J=19.2 Hz, 3H), 1.09 (t, J=7.2 Hz, 3H). LC-MS (ESI, Method 4) t_{R}=2.04 min, m/z (M+H)⁺=466.4.

### Example 33

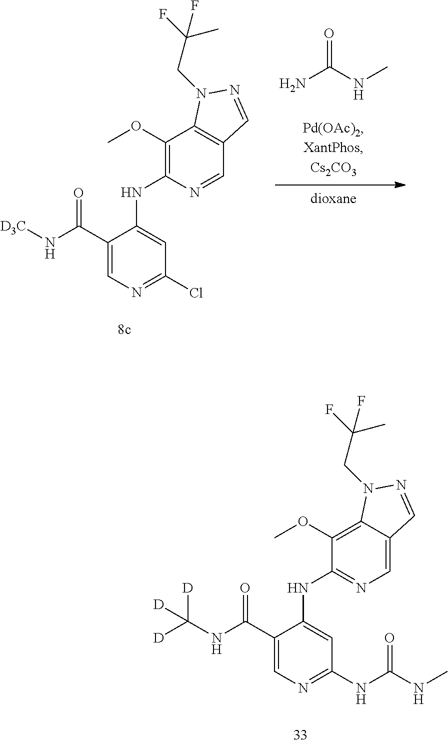

### 4-((1-(2,2-Difluoropropyl)-7-methoxy-1H-pyrazolo[4,3-c]pyridin-6-yl)amino)-N-(methyl-d3)-6-(3-methylureido)nicotinamide (33)

To a solution of 8c (20 mg, 0.048 mmol), Pd(OAc)₂ (2 mg, 0.01 mmol), XantPhos (11 mg, 0.02 mmol) in dioxane (3 mL) was added 1-methylurea (18 mg, 0.24 mmol) at r.t. The reaction was stirred at 100° C. for 5 h under nitrogen protection. The resulting solution was added into H₂O (10 mL) and extracted by EA (20 mL). The combined organic layer was washed by brine (10 mL*2), dried over Na₂SO₄ and filtered. The filtrate was concentrated in vacuo. The crude was purified by prep-HPLC (Method E) to afford 33 (1.2 mg, 6% yield) as a light-yellow solid. ¹H NMR (400 MHz, DMSO-d₆) δ 11.62 (s, 1H), 9.33 (s, 1H), 8.69 (s, 1H), 8.61 (s, 1H), 8.49 (s, 1H), 8.46 (s, 1H), 8.35 (s, 1H), 8.25-8.16 (m, 1H), 4.93 (t, J=12.8 Hz, 2H), 3.90 (s, 3H), 2.73 (d, J=4.4 Hz, 3H), 1.70 (t, J=19.2 Hz, 3H). LC-MS (ESI, Method 4) t_{R}=1.88 min, m/z (M+H)⁺=452.3.

### Example 34

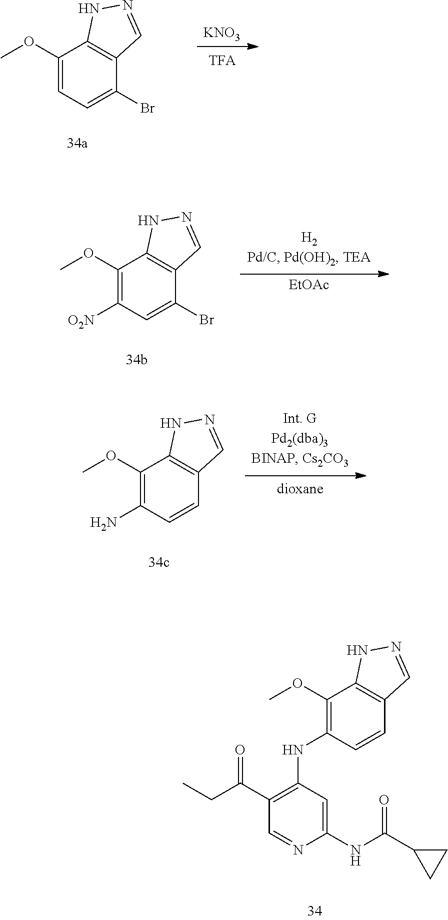

### Step 1. 4-Bromo-7-methoxy-6-nitro-1H-indazole (34b)

To a solution of 34a (400 mg, 1.76 mmol) in TFA (10 mL) was added KNO₃ (178 mg, 1.76 mmol) at r.t. The mixture was stirred at 70° C. for 1 h. After cooling to r.t., TFA was removed by pumping through N₂. The residue was dissolved in DCM (30 mL) and basified with sat. Na₂CO₃ to pH=9. The mixture was extracted with EtOAc (200 mL*3). The combined organic phase was concentrated, and the residue was purified by flash chromatography on silica gel (PE/EA=4/1) to afford 34b (450 mg, 94% yield) as a yellow solid. ¹H NMR (400 MHz, DMSO-d₆) δ 14.46 (s, 1H), 8.28 (s, 1H), 7.82 (s, 1H), 3.95 (s, 3H).

### Step 2. 7-Methoxy-1H-indazol-6-amine (34c)

A mixture of 34b (450 mg, 1.65 mmol), Pd/C (100 mg, palladium 10% on carbon wetted with 50% water), Pd(OH)₂ (100 mg, palladium hydroxide 10% on carbon wetted with ca. 55% water) and TEA (167 mg, 1.65 mmol, 0.23 mL) in EtOAc (8 mL) and MeOH (2 mL) was stirred at 30° C. for 3 h under H₂ (1 atom) atmosphere. The mixture was filtered and the filtrate was concentrated to afford 33c (270 mg, yield given) as a brown solid. LC-MS (ESI, Method 3) t_{R}=0.72 min, m/z (M+H)⁺=164.3.

### Step 3. N-(4-((7-methoxy-1H-indazol-6-yl)amino)-5-propionylpyridin-2-yl)cyclopropanecarboxamide (34)

A mixture of Int. G (30 mg, 0.12 mmol), 34c (58 mg, 0.14 mmol), Pd₂(dba)₃ (9 mg, 0.009 mmol), BINAP (8 mg, 0.011 mmol) and Cs₂CO₃ (77 mg, 0.24 mmol) in dioxane (1 mL) was stirred at 115° C. for 16 h under N₂ atmosphere. The reaction mixture was concentrated and the residue was purified by prep-TLC (DCM/MeOH=20/1) to afford 34 (10 mg, 22% yield) as a yellow solid. ¹H NMR (400 MHz, DMSO-d₆) δ 13.39 (s, 1H), 10.90 (s, 1H), 10.85 (s, 1H), 8.85 (s, 1H), 8.11 (s, 1H), 7.70 (s, 1H), 7.52 (d, J=8.4 Hz, 1H), 7.08 (d, J=8.4 Hz, 1H), 3.89 (s, 3H), 3.12 (q, J=7.2 Hz, 2H), 1.99-1.95 (m, 1H), 1.13 (t, J=7.2 Hz, 3H), 0.76-0.72 (m, 4H). LC-MS (ESI, Method 2) t_{R}=1.10 min, m/z (M+H)⁺=380.0.

### Example 35

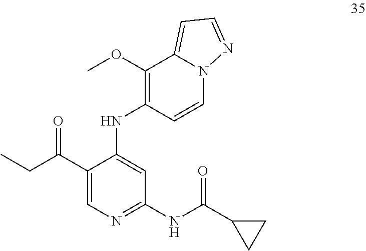 [SMILES: CCC(=O)c1cnc(NC(=O)C2CC2)cc1Nc1ccn2nccc2c1OC]

### N-[4-[(4-methoxypyrazolo[1,5-a]pyridin-5-yl)amino]-5-propanoyl-2-pyridyl]cyclopropanecarboxamide (35)

The synthesis of compound 35 refers to the patent (CN115466257A, page 24-26). LC-MS (ESI, Method 4) t_{R}=0.72 min, m/z (M+H)⁺=380.2. ¹H NMR (400 MHz, CDCl₃): δ 11.02 (brs, 1H), 8.71 (s, 1H), 8.42 (brs, 1H), 8.29 (d, J=7.2 Hz, 1H), 7.93 (s, 1H), 7.90 (d, J=2.0 Hz, 1H), 6.87 (d, J=7.2 Hz, 1H), 6.61 (d, J=2.0 Hz, 1H), 4.00 (s, 3H), 3.03 (q, J=7.2 Hz, 2H), 1.58-1.49 (m, 1H), 1.26 (t, J=7.2 Hz, 3H), 1.09-1.03 (m, 2H), 0.92-0.85 (m, 2H).

### Example 36

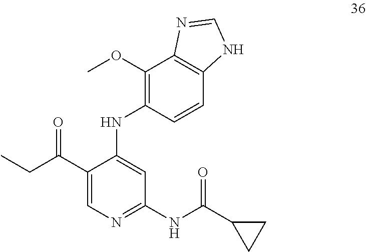 [SMILES: CCC(=O)c1cnc(NC(=O)C2CC2)cc1Nc1ccc2[nH]cnc2c1OC]

### N-[4-[(4-methoxy-1H-benzimidazol-5-yl)amino]-5-propanoyl-2-pyridyl]cyclopropanecarboxamide (36)

The synthesis of compound 36 refers to the patent (CN115466257A, page 33-34). LC-MS (ESI, Method 4) t_{R}=0.52 min, m/z (M+H)⁺=380.2. ¹H NMR (400 MHz, CDCl₃): δ 10.98 (s, 1H), 9.82 (brs, 1H), 8.61 (s, 1H), 8.61 (s, 1H), 7.94 (s, 1H), 7.70 (s, 1H), 7.23-7.13 (m, 2H), 4.28 (s, 3H), 3.03 (q, J=7.2 Hz, 2H), 1.72-1.62 (m, 1H), 1.27 (t, J=7.2 Hz, 3H), 1.03-0.95 (m, 2H), 0.91-0.82 (m, 2H).

### Example 37

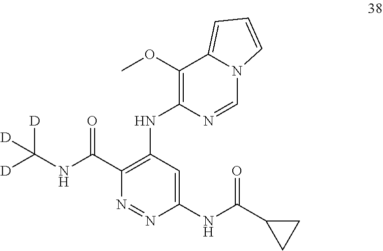 [SMILES: [2H]C([2H])([2H])NC(=O)c1nnc(NC(=O)C2CC2)cc1Nc1ncn2cccc2c1OC]

### 6-(cyclopropanecarboxamido)-4-((4-methoxypyrrolo[1,2-c]pyrimidin-3-yl)amino)-N-(methyl-d3)pyridazine-3-carboxamide (37)

The synthesis of compound 37 refers to the patent (CN116262739A, page 35-36). LC-MS (ESI, Method 4) t_{R}=0.76 min, m/z (M+H)⁺=385.2. ¹H NMR (400 MHz, DMSO-d₆): S 11.50 (s, 1H), 11.24 (s, 1H), 9.14 (s, 1H), 9.01 (s, 1H), 8.99 (d, J=0.8 Hz, 1H), 7.67 (dd, J=2.8, 1.2 Hz, 1H), 6.90 (dd, J=3.6, 2.8 Hz, 1H), 6.56-6.53 (m, 1H), 3.99 (s, 3H), 2.13-2.07 (m, 1H), 0.84 (d, J=6.4 Hz, 4H).

A Summary of representative compounds are shown in Table 1A.

TABLE 1A
Exemplary Compounds

| Example | Structure | Name | Mol Weight |
|---|---|---|---|
| 1 | 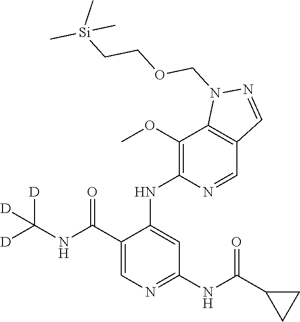 [SMILES: [2H]C([2H])([2H])NC(=O)c1cnc(NC(=O)C2CC2)cc1Nc1ncc2cnn(COCC[Si](C)(C)C)c2c1OC] | 6-(Cyclopropanecarboxamido)-4- ((7-methoxy-1-((2- (trimethylsilyl)ethoxy)methyl)-1H- pyrazolo[4,3-c]pyridin-6- yl)amino)-N-(methyl- d3)nicotinamide | 514.67 |
| 2 | 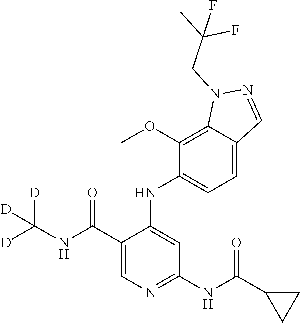 [SMILES: [2H]C([2H])([2H])NC(=O)c1cnc(NC(=O)C2CC2)cc1Nc1ccc2cnn(CC(C)(F)F)c2c1OC] | 6-(Cyclopropanecarboxamido)-4- ((1-(2,2-difluoropropyl)-7- methoxy-1H-indazol-6-yl)amino)- N-(methyl-d3)nicotinamide | 461.48 |
| 3 | 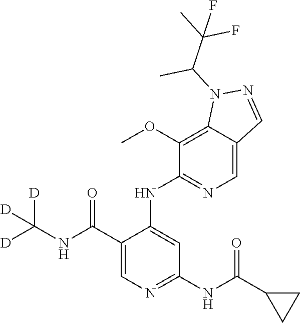 [SMILES: [2H]C([2H])([2H])NC(=O)c1cnc(NC(=O)C2CC2)cc1Nc1ncc2cnn(C(C)C(C)(F)F)c2c1OC] | 6-(Cyclopropanecarboxamido)-4- ((7-methoxy-1-(1,1,1- trifluoropropan-2-yl)-1H- pyrazolo[4,3-c]pyridin-6- yl)amino)-N-(methyl- d3)nicotinamide | 480.46 |
| 3A | 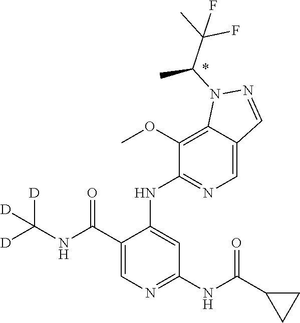 [SMILES: [2H]C([2H])([2H])NC(=O)c1cnc(NC(=O)C2CC2)cc1Nc1ncc2cnn([C@@H](C)C(C)(F)F)c2c1OC] | (R*)-6- (Cyclopropanecarboxamido)-4-((7- methoxy-1-(1,1,1-trifluoropropan- 2-yl)-1H-pyrazolo[4,3-c]pyridin-6- yl)amino)-N-(methyl- d3)nicotinamide | 480.46 |
| 3B | 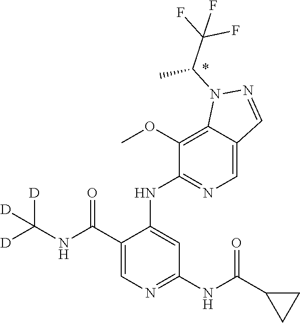 [SMILES: [2H]C([2H])([2H])NC(=O)c1cnc(NC(=O)C2CC2)cc1Nc1ncc2cnn([C@H](C)C(F)(F)F)c2c1OC] | (S*)-6- (Cyclopropanecarboxamido)-4-((7- methoxy-1-(1,1,1-trifluoropropan- 2-yl)-1H-pyrazolo[4,3-c]pyridin-6- yl)amino)-N-(methyl- d3)nicotinamide | 480.46 |
| 4 | 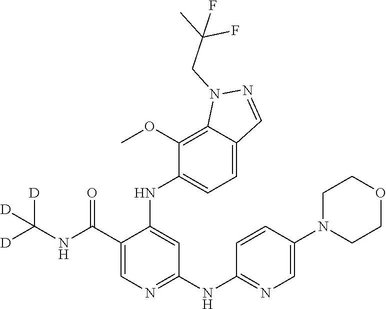 [SMILES: [2H]C([2H])([2H])NC(=O)c1cnc(Nc2ccc(N3CCOCC3)cn2)cc1Nc1ccc2cnn(CC(C)(F)F)c2c1OC] | 4-((1-(2,2-Difluoropropyl)-7- methoxy-1H-indazol-6-yl)amino)- N-(methyl-d3)-6-((5- morpholinopyridin-2- yl)amino)nicotinamide | 555.59 |
| 5 | 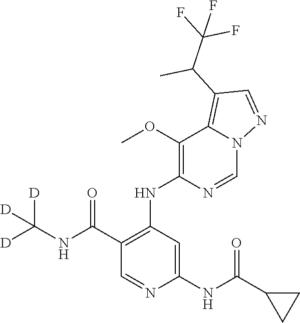 [SMILES: [2H]C([2H])([2H])NC(=O)c1cnc(NC(=O)C2CC2)cc1Nc1ncn2ncc(C(C)C(F)(F)F)c2c1OC] | 6-(Cyclopropanecarboxamido)-4- ((4-methoxy-3-(1,1,1- trifluoropropan-2-yl)pyrazolo[1,5- c]pyrimidin-5-yl)amino)-N- (methyl-d3)nicotinamide | 480.46 |
| 5A | 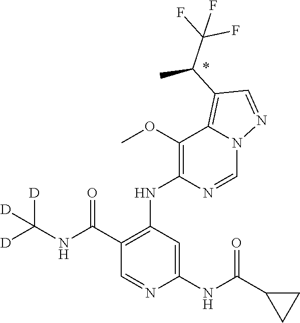 [SMILES: [2H]C([2H])([2H])NC(=O)c1cnc(NC(=O)C2CC2)cc1Nc1ncn2ncc([C@@H](C)C(F)(F)F)c2c1OC] | (R*)6- (Cyclopropanecarboxamido)-4-((4- methoxy-3-(1,1,1-trifluoropropan- 2-yl)pyrazolo[1,5-c]pyrimidin-5- yl)amino)-N-(methyl- d3)nicotinamide | 480.46 |
| 5B | 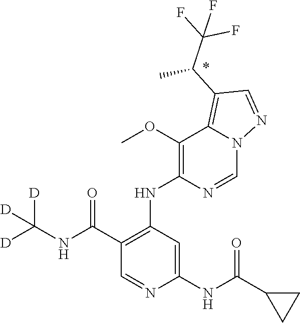 [SMILES: [2H]C([2H])([2H])NC(=O)c1cnc(NC(=O)C2CC2)cc1Nc1ncn2ncc([C@H](C)C(F)(F)F)c2c1OC] | (S*)6- (Cyclopropanecarboxamido)-4-((4- methoxy-3-(1,1,1-trifluoropropan- 2-yl)pyrazolo[1,5-c]pyrimidin-5- yl)amino)-N-(methyl- d3)nicotinamide | 480.46 |
| 6 | 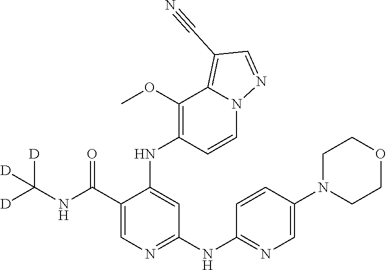 [SMILES: [2H]C([2H])([2H])NC(=O)c1cnc(Nc2ccc(N3CCOCC3)cn2)cc1Nc1ccn2ncc(C#N)c2c1OC] | 4-((3-Cyano-4- methoxypyrazolo[1,5-a]pyridin-5- yl)amino)-N-(methyl-d3)-6-((5- morpholinopyridin-2- yl)amino)nicotinamide | 502.54 |
| 7 | 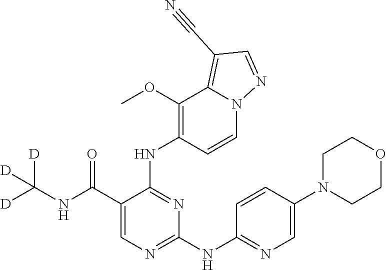 [SMILES: [2H]C([2H])([2H])NC(=O)c1cnc(Nc2ccc(N3CCOCC3)cn2)nc1Nc1ccn2ncc(C#N)c2c1OC] | 4-((3-Cyano-4- methoxypyrazolo[1,5-a]pyridin-5- yl)amino)-N-(methyl-d3)-2-((5- morpholinopyridin-2- yl)amino)pyrimidine-5- carboxamide | 503.53 |
| 8 | 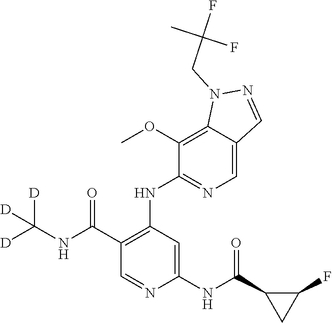 [SMILES: [2H]C([2H])([2H])NC(=O)c1cnc(NC(=O)[C@@H]2C[C@@H]2F)cc1Nc1ncc2cnn(CC(C)(F)F)c2c1OC] | 4-((1-(2,2-Difluoropropyl)-7- methoxy-1H-pyrazolo[4,3- c]pyridin-6-yl)amino)-6-((1S,2S)- 2-fluorocyclopropane-1- carboxamido)-N-(methyl- d3)nicotinamide | 480.46 |
| 9 | 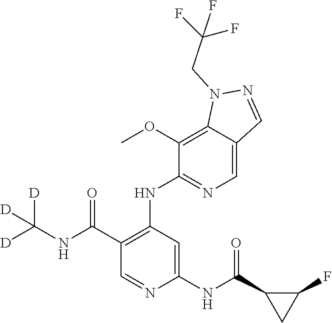 [SMILES: [2H]C([2H])([2H])NC(=O)c1cnc(NC(=O)[C@@H]2C[C@@H]2F)cc1Nc1ncc2cnn(CC(F)(F)F)c2c1OC] | 6-((1S,2S)-2-fluorocyclopropane- 1-carboxamido)-4-((7-methoxy-1- (2,2,2-trifluoroethyl)-1H- pyrazolo[4,3-c]pyridin-6- yl)amino)-N-(methyl- d3)nicotinamide | 484.42 |
| 10 | 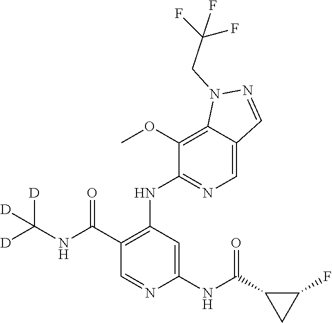 [SMILES: [2H]C([2H])([2H])NC(=O)c1cnc(NC(=O)[C@H]2C[C@H]2F)cc1Nc1ncc2cnn(CC(F)(F)F)c2c1OC] | 6-((1R,2R)-2-fluorocyclopropane- 1-carboxamido)-4-((7-methoxy-1- (2,2,2-trifluoroethyl)-1H- pyrazolo[4,3-c]pyridin-6- yl)amino)-N-(methyl- d3)nicotinamide | 484.42 |
| 11 | 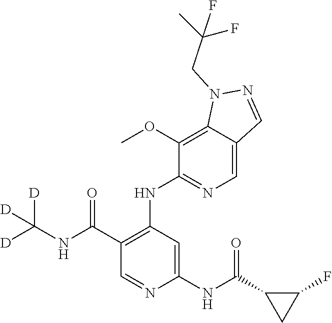 [SMILES: [2H]C([2H])([2H])NC(=O)c1cnc(NC(=O)[C@H]2C[C@H]2F)cc1Nc1ncc2cnn(CC(C)(F)F)c2c1OC] | 4-((1-(2,2-Difluoropropyl)-7- methoxy-1H-pyrazolo[4,3- c]pyridin-6-yl)amino)-6-((1R,2R)- 2-fluorocyclopropane-1- carboxamido)-N-(methyl- d3)nicotinamide | 480.46 |
| 12 | 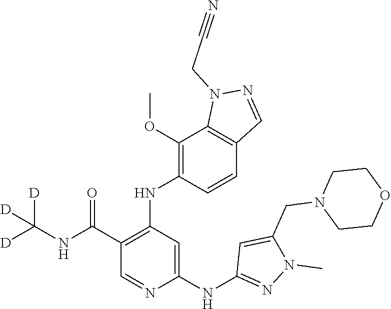 [SMILES: [2H]C([2H])([2H])NC(=O)c1cnc(Nc2cc(CN3CCOCC3)n(C)n2)cc1Nc1ccc2cnn(CC#N)c2c1OC] | 4-((1-(Cyanomethyl)-7-methoxy- 1H-indazol-6-yl)amino)-N- (methyl-d3)-6-((1-methyl-5- (morpholinomethyl)-1H-pyrazol- 3-yl)amino)nicotinamide | 533.60 |
| 13 | 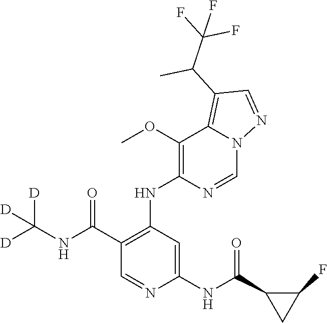 [SMILES: [2H]C([2H])([2H])NC(=O)c1cnc(NC(=O)[C@@H]2C[C@@H]2F)cc1Nc1ncn2ncc(C(C)C(F)(F)F)c2c1OC] | 6-((1S,2S)-2-fluorocyclopropane- 1-carboxamido)-4-((4-methoxy-3- (1,1,1-trifluoropropan-2- yl)pyrazolo[1,5-c]pyrimidin-5- yl)amino)-N-(methyl- d3)nicotinamide | 498.45 |
| 14 | 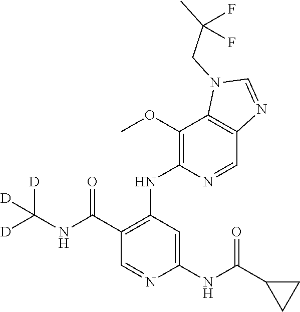 [SMILES: [2H]C([2H])([2H])NC(=O)c1cnc(NC(=O)C2CC2)cc1Nc1ncc2ncn(CC(C)(F)F)c2c1OC] | 6-(Cyclopropanecarboxamido)-4- ((1-(2,2-difluoropropyl)-7- methoxy-1H-imidazo[4,5- c]pyridin-6-yl)amino)-N-(methyl- d3)nicotinamide | 462.47 |
| 15 | 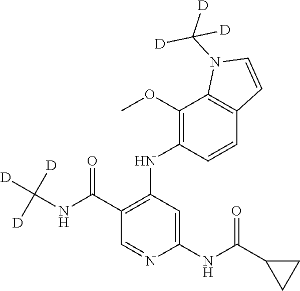 [SMILES: [2H]C([2H])([2H])NC(=O)c1cnc(NC(=O)C2CC2)cc1Nc1ccc2ccn(C([2H])([2H])[2H])c2c1OC] | 6-(Cyclopropanecarboxamido)-4- ((7-methoxy-1-(methyl-d3)-1H - indol-6-yl)amino)-N-(methyl- d3)nicotinamide | 399.48 |
| 16 | 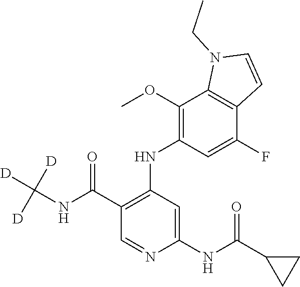 [SMILES: [2H]C([2H])([2H])NC(=O)c1cnc(NC(=O)C2CC2)cc1Nc1cc(F)c2ccn(CC)c2c1OC] | 6-(Cyclopropanecarboxamido)-4- ((1-ethyl-4-fluoro-7-methoxy-1H- indol-6-yl)amino)-N-(methyl- d3)nicotinamide | 428.47 |
| 17 | 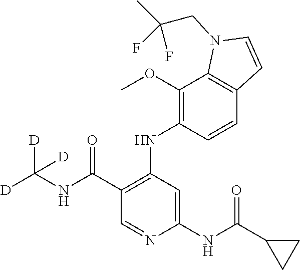 [SMILES: [2H]C([2H])([2H])NC(=O)c1cnc(NC(=O)C2CC2)cc1Nc1ccc2ccn(CC(C)(F)F)c2c1OC] | 6-(Cyclopropanecarboxamido)-4- ((1-(2,2-difluoropropyl)-7- methoxy-1H-indol-6-yl)amino)-N- (methyl-d3)nicotinamide | 460.49 |
| 18 | 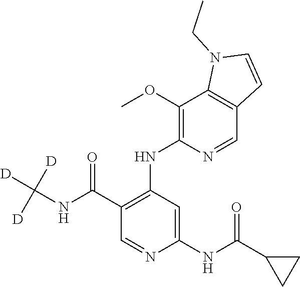 [SMILES: [2H]C([2H])([2H])NC(=O)c1cnc(NC(=O)C2CC2)cc1Nc1ncc2ccn(CC)c2c1OC] | 6-(Cyclopropanecarboxamido)-4- ((1-ethyl-7-methoxy-1H- pyrrolo[3,2-c] pyridin-6-yl) amino)-N-(methyl- d3)nicotinamide | 411.47 |
| 19 | 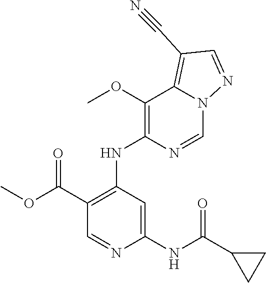 [SMILES: COC(=O)c1cnc(NC(=O)C2CC2)cc1Nc1ncn2ncc(C#N)c2c1OC] | Methyl 4-((3-cyano-4- methoxypyrazolo[1,5-c]pyrimidin- 5-yl)amino)-6- (cyclopropanecarboxamido) nicotinate | 407.38 |
| 20 | 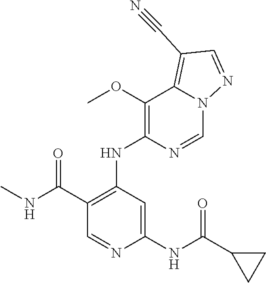 [SMILES: CNC(=O)c1cnc(NC(=O)C2CC2)cc1Nc1ncn2ncc(C#N)c2c1OC] | 4-((3-Cyano-4- methoxypyrazolo[1,5-c]pyrimidin- 5-yl)amino)-6- (cyclopropanecarboxamido)-N- methylnicotinamide | 406.40 |
| 21 | 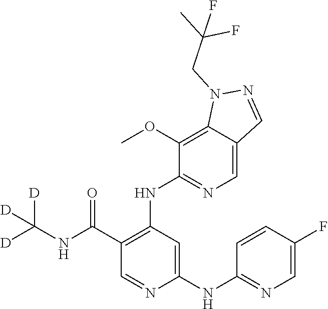 [SMILES: [2H]C([2H])([2H])NC(=O)c1cnc(Nc2ccc(F)cn2)cc1Nc1ncc2cnn(CC(C)(F)F)c2c1OC] | 4-((1-(2,2-Difluoropropyl)-7- methoxy-1H-pyrazolo[4,3- c]pyridin-6-yl)amino)-6-((5- fluoropyridin-2-yl)amino)-N- (methyl-d3)nicotinamide | 489.47 |
| 22 | 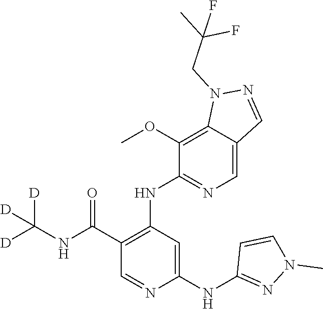 [SMILES: [2H]C([2H])([2H])NC(=O)c1cnc(Nc2ccn(C)n2)cc1Nc1ncc2cnn(CC(C)(F)F)c2c1OC] | 4-((1-(2,2-Difluoropropyl)-7- methoxy-1H-pyrazolo[4,3- c]pyridin-6-yl)amino)-N-(methyl- d3)-6-((1-methyl-1H-pyrazol-3- yl)amino)nicotinamide | 474.48 |
| 23 | 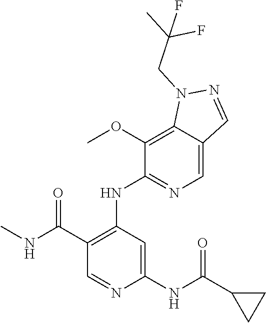 [SMILES: CNC(=O)c1cnc(NC(=O)C2CC2)cc1Nc1ncc2cnn(CC(C)(F)F)c2c1OC] | 6-(Cyclopropanecarboxamido)-4- ((1-(2,2-difluoropropyl)-7- methoxy-1H-pyrazolo[4,3- c]pyridin-6-yl)amino)-N- methylnicotinamide | 459.45 |
| 24 | 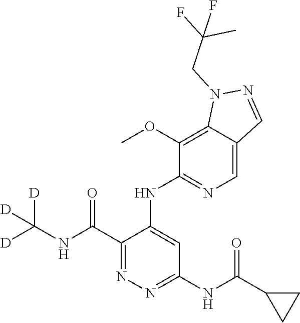 [SMILES: [2H]C([2H])([2H])NC(=O)c1nnc(NC(=O)C2CC2)cc1Nc1ncc2cnn(CC(C)(F)F)c2c1OC] | 6-(Cyclopropanecarboxamido)-4- ((1-(2,2-difluoropropyl)-7- methoxy-1H-pyrazolo[4,3- c]pyridin-6-yl)amino)-N-(methyl- d3)pyridazine-3-carboxamide | 463.46 |
| 25 | 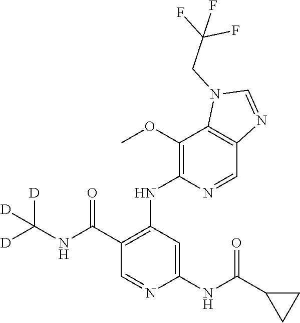 [SMILES: [2H]C([2H])([2H])NC(=O)c1cnc(NC(=O)C2CC2)cc1Nc1ncc2ncn(CC(F)(F)F)c2c1OC] | 6-(Cyclopropanecarboxamido)-4- ((7-methoxy-1-(2,2,2- trifluoroethyl)-1H-imidazo[4,5- c]pyridin-6-yl)amino)-N-(methyl- d3)nicotinamide | 466.43 |
| 26 | 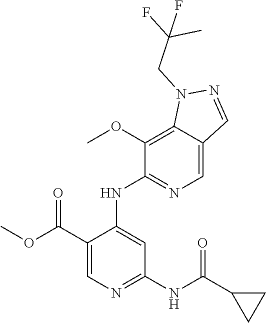 [SMILES: COC(=O)c1cnc(NC(=O)C2CC2)cc1Nc1ncc2cnn(CC(C)(F)F)c2c1OC] | Methyl 6- (cyclopropanecarboxamido)-4-((1- (2,2-difluoropropyl)-7-methoxy- 1H-pyrazolo[4,3-c]pyridin-6- yl)amino)nicotinate | 460.43 |
| 27 | 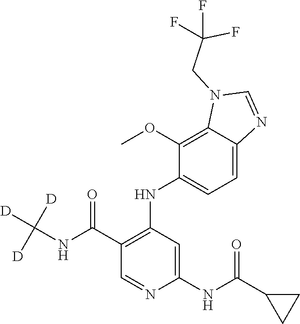 [SMILES: [2H]C([2H])([2H])NC(=O)c1cnc(NC(=O)C2CC2)cc1Nc1ccc2ncn(CC(F)(F)F)c2c1OC] | 6-(cyclopropanecarboxamido)-4- ((7-methoxy-1-(2,2,2- trifluoroethyl)-1H- benzo[d]imidazol-6-yl)amino)-N- (methyl-d3)nicotinamide | 465.44 |
| 28 | 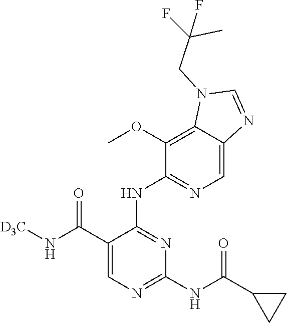 [SMILES: [2H]C([2H])([2H])NC(=O)c1cnc(NC(=O)C2CC2)nc1Nc1ncc2ncn(CC(C)(F)F)c2c1OC] | 2-(cyclopropanecarboxamido)-4- ((1-(2,2-difluoropropyl)-7- methoxy-1H-pyrazolo[4,3- c]pyridin-6-yl)amino)-N-(methyl- d3)pyrimidine-5-carboxamide | 463.46 |
| 29 | 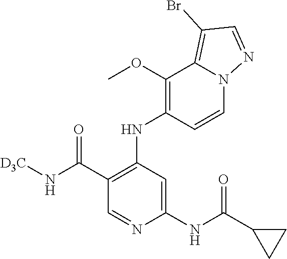 [SMILES: [2H]C([2H])([2H])NC(=O)c1cnc(NC(=O)C2CC2)cc1Nc1ccn2ncc(Br)c2c1OC] | 4-((3-bromo-4- methoxypyrazolo[1,5-a]pyridin-5- yl)amino)-6- (cyclopropanecarboxamido)-N- (methyl-d3)nicotinamide | 462.31 |
| 30 | 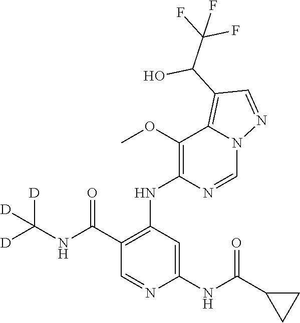 [SMILES: [2H]C([2H])([2H])NC(=O)c1cnc(NC(=O)C2CC2)cc1Nc1ncn2ncc(C(O)C(F)(F)F)c2c1OC] | 6-(cyclopropanecarboxamido)-4- ((4-methoxy-3-(2,2,2-trifluoro-1- hydroxyethyl)pyrazolo[1,5- c]pyrimidin-5-yl)amino)-N- (methyl-d3)nicotinamide | 482.43 |
| 31 | 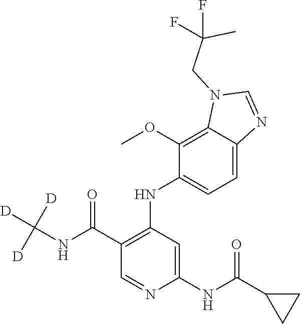 [SMILES: [2H]C([2H])([2H])NC(=O)c1cnc(NC(=O)C2CC2)cc1Nc1ccc2ncn(CC(C)(F)F)c2c1OC] | 6-(cyclopropanecarboxamido)-4- ((1-(2,2-difluoropropyl)-7- methoxy-1H-benzo[d]imidazol-6- yl)amino)-N-(methyl- d3)nicotinamide | 461.48 |
| 32 | 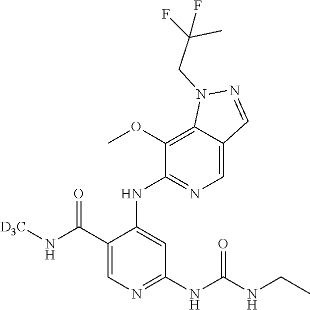 [SMILES: [2H]C([2H])([2H])NC(=O)c1cnc(NC(=O)NCC)cc1Nc1ncc2cnn(CC(C)(F)F)c2c1OC] | 4-((1-(2,2-difluoropropyl)-7- methoxy-1H-pyrazolo[4,3- c]pyridin-6-yl)amino)-6-(3- ethylureido)-N-(methyl- d3)nicotinamide | 465.48 |
| 33 | 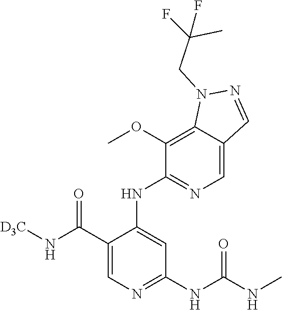 [SMILES: [2H]C([2H])([2H])NC(=O)c1cnc(NC(=O)NC)cc1Nc1ncc2cnn(CC(C)(F)F)c2c1OC] | 4-((1-(2,2-difluoropropyl)-7- methoxy-1H-pyrazolo[4,3- c]pyridin-6-yl)amino)-N-(methyl- d3)-6-(3- methylureido)nicotinamide | 451.45 |

Note:
A asterisk * at the chiral center in the chemical structures or the S* or R* in the names means the compound is a chiral pure but absolute configuration unknown single isomer.

Binding Activity test for JH2 domain of JAK and TYK2

The compounds are prepared in DMSO for 200× top dose, then serial dilute it with 27-fold for 3 more points. Add 8 μL/well in echo source plate, echo will add 75 nL/well with 3-fold serial dilution for 11 points in assay plate. 75 nL DMSO for high control and low control.

Prepare 3× working solutions (1.5 nM) of JAK1-JH2 domain or TYK2-JH2 domain enzyme in assay buffer, add 5 μL of enzyme working solutions per well to the assay plate including high control. For low control, add 5 μL/well assay buffer. And then spin down at 1000 rpm and centrifuge for 30 seconds. After enzyme system prepared, add 5 μL of Tb-antibody solution into each wells of assay plate. Spin down at 1000 rpm and centrifuge for 30 sec. After Tb-antibody added, also add 5 μL of tracer into the assay plate. Spin down at 1000 rpm and centrifuge for 30 sec. Incubate for 60 mins at 25° C. firstly and then continue to incubate the plate at 4° C. overnight. Read by envision in FRET mode. The Luminescence value was recorded by a multi-label reader Envision (PerkinElmer). Inhibition Rate was calculated relative to vehicle (DMSO) treated control wells using following formula:

$$\% Inhibition = 100 - \frac{{RLU}_{compound} - RLU low control}{RLU high control - RLU low control}$$

wherein
RLU_compound=the relative light unit of cells treated with test compounds
RLU_low control=the relative light unit of medium with DMSO only
RLU_high control=the relative light unit of cells treated with DMSO only

The dose-response (percent inhibition) curve was plotted and IC50 values (the concentration that causes 50% growth inhibition) were determined by GraphPad software. The IC50 of tested compounds are shown in Table 2.

TABLE 2
IC50 on JH2 domain activity (nM)

| | TYK2 JH2 | JAK1 JH2 | |
|---|---|---|---|
| | Binding | Binding | JAK1/TYK2 |
| Example | IC50(nM) | IC50(nM) | selectivity |
| 1 | 9.309 | >298.5 | >32.07 |
| 2 | 0.239 | 18.873 | 78.97 |
| 3 | 0.682 | 121.087 | 177.55 |
| 3A | 8.544 | >298.5 | >34.94 |
| 3B | 0.805 | 113.795 | 141.36 |
| 4 | 0.165 | 8.453 | 51.23 |
| 5 | 0.271 | 160.049 | 590.59 |
| 5A | 1.511 | >298.5 | >197.55 |
| 5B | 0.197 | 141.061 | 716.05 |
| 6 | 0.157 | 0.703 | 4.48 |
| 7 | 0.404 | 10.746 | 26.60 |
| 8 | 0.288 | 161.874 | 561.99 |
| 9 | 0.212 | 35.209 | 166.08 |
| 10 | 0.408 | 35.105 | 86.04 |
| 11 | 0.325 | 73.828 | 227.16 |
| 12 | 0.189 | 1.616 | 8.55 |
| 13 | 1.702 | >298.5 | >175.38 |
| 14 | 0.178 | 38.779 | 217.86 |
| 15 | 0.094 | 1.47 | 15.64 |
| 16 | 0.083 | 5.818 | 70.10 |
| 17 | 0.341 | 41.464 | 121.60 |
| 18 | 0.049 | 4.094 | 83.55 |
| 19 | 1.634 | 22.069 | 13.51 |
| 20 | 0.304 | 19.339 | 63.62 |
| 21 | 0.396 | 139.817 | 353.07 |
| 22 | 2.623 | 297.209 | 113.31 |
| 23 | 0.315 | 125.589 | 398.70 |
| 24 | 0.271 | 21.643 | 79.86 |
| 25 | 0.174 | 46.721 | 268.51 |
| 26 | 0.26 | 115.263 | 443.32 |
| 27 | 2.768 | >298.5 | >107.8 |
| 28 | >298.5 | >298.5 | / |
| 29 | 0.188 | 7.870 | 42.0 |
| 30 | 0.134 | 110.154 | 822.4 |
| 31 | 0.300 | 20.772 | 69.3 |
| 32 | 1.702 | >298.5 | >175.4 |
| 33 | 2.136 | >298.5 | >139.8 |
| 34 | 3.940 | 31.670 | 8.0 |
| BMS986165 | 0.133 | 0.631 | 4.7 |
| 35 | 2.96 | 67.124 | 22.68 |
| 36 | 2.716 | 17.402 | 6.41 |
| 37 | 1.48 | 10.36 | 7.0 |

BMS986165, See, PCT/CN2022/139649 (filed on Dec. 16, 2022), Example 41.

Exemplary compounds showed good selectivity over JAK1. These compounds can be used to eliminate the side effects caused by JAK1 inhibition in autoimmune disease.

### Biochemical Assay

### Testing for JAK1, JAK2 and TYK2 Kinase Activities

JAK activity was determined in the reaction buffer 50 mM HEPES, 0.01% Brij35, 10 mM MgCl₂, 2 mM DTT by a microfluidic assay. The phosphorylation of a FAM labeled peptide substrate was monitored in the Caliper EZ Reader II (Perkin Elmer). The assay condition for each batch of enzyme (Carna Biosciences) was optimized to obtain 10% conversion rate of peptide substrate.

The test compounds were dissolved in DMSO to a stock concentration of 10 mM. Three-fold serially diluted compounds with top concentration of 5 μM were pre-incubated with JAK1, JAK2 or TYK2 for 10 min at ambient temperature. The final DMSO concentration of assay mixture was 1%. FAM labeled peptide substrate (final concentration 3 μM) and ATP (Km concentration or 1 mM) were sequentially added to initiate the kinase reaction at 28° C. for 90 min (JAK), 15 min (JAK2) and 30 min (TYK2), respectively. The reaction was stopped by adding 50 mM EDTA.

The well in the test plate without enzyme was defined as 100% inhibition. And the well without compound but with equivalent DMSO was defined as no inhibition. The percent inhibition was calculated by the following formula:

$$\% Inhibition = \frac{Conversion \% \_max - Conversion \% \_sample}{C o n v e rsion \% \_max - Conversion \% \_min} \times 100$$

wherein
Conversion %_ₘₐₓ=the conversion rate in the positive well without addition of compound
Conversion %_ₛₐₘₚₗₑ=the conversion rate of test compounds
Conversion %_ₘᵢₙ=the conversion rate in the well without addition of enzyme

The dose-response (percent inhibition) curve was plotted and IC50 values were determined by GraphPad software. The IC50 values of tested compounds were list in Table 3.

TABLE 3
JAK1, JAK2 and TYK2 IC50 Values of Illustrative Compounds

| | JAK1 | JAK2 | TYK2 |
|---|---|---|---|
| | (10 nM) (1 mM | (1.5 nM) (1 mM | (20 nM) (1 mM |
| Example | ATP), nM | ATP), nM | ATP), nM |
| 4 | >20000 | >20000 | >20000 |
| 23 | >20000 | >20000 | >20000 |
| 24 | >20000 | >20000 | >20000 |
| 276* | >20000 | >20000 | >20000 |

Example 276*: See, PCT/CN2022/139649 (filed on Dec. 16, 2022), Example 276.

Exemplary compounds showed excellent selectivity over JAK1, JAK2 and TYK2 kinase domain inhibition.

### Anti-Proliferative Assay

Dimerization domain of Tel protein fused with JAK kinase domain was permanently transduced into Ba/F3 cells, whose proliferation is dependent on JAK activity in the absence of IL-3 induction. These engineered Ba/F3-FL-TYK2-P760L cells were used to monitor JAK inhibitory activities of the compounds in the cellular. Ba/F3 cells were cultured in RPMI-1640 (Corning) containing 10% fetal bovine serum. Cells were seeded at 2000/well of white flat bottom 96-well plates. The well containing medium only was used as background control. After 24 h growth, cells were treated with compounds. The test compounds were dissolved in DMSO to a stock concentration of 20 mM. 3-fold serially diluted compounds for 9 concentrations with top concentration of 20 μM was added into the each well. The final DMSO concentration was 0.1%. The cells continued to grow at 37° C. in 5% CO₂ for 72 h after compound treatment. The viability was measured by cellular ATP determination using the Cell-Titer Glo luciferase reagent (Promega). The Luminescence value was recorded by a multi-label reader Envision (PerkinElmer). Inhibition Rate was calculated relative to vehicle (DMSO) treated control wells using following formula:

$$\% Inhibition = 100 - \frac{RLU\_compound - RLU\_b1ank}{RLU\_contro1 - RLU\_b1ank} \times 100 \%$$

wherein
RLU_compound=the relative light unit of cells treated with test compounds
RLU_blank=the relative light unit of medium with DMSO only
RLU_control=the relative light unit of cells treated with DMSO only

The dose-response (percent inhibition) curve was plotted and IC50 values (the concentration that causes 50% growth inhibition) were determined by GraphPad software. The IC50 of tested compounds are shown in Table 4.

TABLE 4
Ba/F3-FL-TYK2-P760L Cells IC50
Values of Exemplary Compounds

| | Baf3-FL-T2P760L |
|---|---|
| Example | (nM) |
| 1 | 442.3 |
| 2 | 86.9 |
| 3 | 164.2 |
| 3A | 1804 |
| 3B | 164.15 |
| 4 | 22.07 |
| 5 | 52.36 |
| 5A | >5000.0 |
| 5B | 62.26 |
| 6 | 231 |
| 7 | 147.22 |
| 8 | 72.69 |
| 9 | 66.75 |
| 10 | 117.85 |
| 11 | 160 |
| 12 | 54.6 |
| 13 | 499.3 |
| 14 | 145.5 |
| 15 | 20.99 |
| 16 | 16.55 |
| 17 | 58.06 |
| 18 | 3.69 |
| 19 | 233.4 |
| 20 | 62.34 |
| 21 | 63.09 |
| 22 | 329.8 |
| 23 | 78.43 |
| 24 | 54.82 |
| 25 | 99 |
| 26 | 726.4 |
| 27 | 128.0 |
| 28 | >5000.0 |
| 29 | 39.51 |
| 30 | 62.86 |
| 31 | 135.20 |
| 32 | / |
| 33 | / |
| 34 | 856.90 |

### IFN-γ Release in NK92 Cells Stimulated by IL-12

Purpose: To evaluate the inhibition activity of TYK2 inhibitors in NK92 cells on TYK2 pathway through ELISA method.

### Materials:

| Items | Vendor | Cat. No. |
|---|---|---|
| PBS | BI | 02-024-01ACS |
| α-MEM | Gibco | 12561056 |
| Inactivated Horse Serum | Gibco | 26050088 |
| FBS | Gibco | 10091148 |
| β- Mercaptoethanol | procell | PB180633 |
| Penicillin-Streptomycin | Gibco | 15140-122 |
| Folic Acid | MCE | HY-16637A |
| i-Inositol | MCE | HY-B1411 |
| Recombinant human IL-2 | PEPROTECH | 200-02-10UG |
| Recombinant human IL-12 P70 | PEPROTECH | 200-12-50UG |
| ELISA MAX ™ Deluxe Set Human | BD | 555142 |
| IFN-γ | | |
| Nunc Edge 2 96-well plate | Thermo | 167425 |
| TMB | Solarbio | PR1200 |
| ELISA Plate | Corning | 9018 |
| Stop Solution | Elabscience | E-ELIR-006 |

Procedure: 1) Seeding NK92 cells into 96-well plate, cell density is 1.5E5 cells/well, 100 uL/well; 2) Add prepared test articles into indicated wells, 50 uL/well, and continue incubate for 1h, at 37° C.; 3) After compound treatment, add 50 uL/well IL-12 into indicated wells, final concentration of IL-12 is 2.5 ng/mL; 4) Incubate cytokine stimulated cells at 37° C. for another 15 hrs in CO₂ incubator; 5) After cytokine stimulation, transfer culture supernatant into a new plate and centrifuge for 5 min at 350× g, and detect IFN-γ concentration in the supernatant with ELISA kit; 6) Data analysis with GrapdPad Prism6.0 software.

TABLE 5
IC50 Values of Exemplary Compounds on TYK2 pathway

| | IC50 on TYK2 pathway |
|---|---|
| Example | (nM) |
| 262* | 26.27 |
| 266* | 37.9 |
| 276* | 27.7 |
| 4 | 14.01 |
| 17 | 21.73 |
| 23 | 17.45 |
| 24 | 8.94 |
| 29 | 32.09 |
| 34 | 922.8 |
| 35 | 1500 |
| 36 | 515.1 |

Example 262*, 266* and 276*: See, PCT/CN2022/139649 (filed on 16 Dec. 2022, incorporated herein by reference), Example 262, 266 and 276, respectively.

Exemplary compounds showed good TYK2 pathway inhibition activity in cells.

### Phosphorylation Inhibition of STAT in Human Whole Blood Testing:

The inhibitory potential of test articles in human whole blood assay was evaluated by the method of flow cytometry.

IFN-alpha will activate JAK1 and TYK2 kinase in T cells by binding to IFN receptors, and then lead to phosphorylation of STAT1 and STAT2. The phosphorylated STAT1 enter nuclear and promote transcription and expression of IFN-gamma. To evaluate the efficacy of TYK2i in T cells, freshly collected human whole blood will be stimulated with certain unit of human IFN-alpha (Universal Type I IFN (1MU), R&D, 11200-2) for 20 mins in incubator. After stimulation, fix cells with Phosflow™ Fix Buffer I (BD, 557870), and then collect fixed cells by centrifugation (500 g, 8 min). Wash cells once with pre-cooled PBS, and then permeabilize cells with cold Perm Buffer III buffer (BD, 558050) for 45 mins on ice. Collect cells by centrifugation (600 g, 8 min) and washed twice with cold PBS. Stain cells with anti-human CD3(FITC Mouse Anti-Human CD3, BD, 555332) and anti-pSTAT1_Y701 (Alexa Fluor 647 Mouse Anti-Stat1 (pY701), BD, 612597) antibodies. Detect pSTAT1 by flow cytometry (CytoFlex S) and analyze data with FlowJo and GraphPad Prism 8 software. IC50 values of test articles were determined using 4-parameter logistic equation.

Exemplary results are summarized in Table 6.

TABLE 6

| | pSTAT1 inhibition in | | pSTAT1 inhibition in |
|---|---|---|---|
| Example | hWB (IC50, nM) | Example | hWB (IC50, nM) |
| 2 | 327.4 | 20 | 166.8 |
| 4 | 151.6 | 276* | 176.6 |
| 9 | 273.6 | 34 | >10000 |
| 15 | 204.9 | 35 | >5000 |
| 16 | 219.7 | 37 | 1469 |

### ADME

### Microsomal Metabolic Stability Assay

Microsomes were pre-incubated with test compound or control compounds for 5 min at 37° C. in 100 mM potassium phosphate buffer, pH 7.4, 1.0 mM EDTA. The reaction was initiated by addition of 15 μL of the NADPH regenerating system to 30 μL of each incubation mixture per time point. The final incubation condition was composed of 0.5 mg/mL microsomal protein, 1 μM test article/positive control, 2 mM NADPH. The 0-minute samples were prepared by addition of a 30 μL aliquot of each incubation mixture to 135 μL quench reagent to precipitate proteins. And then a 15 μL aliquot of the NADPH regenerating system was added. At 5, 15, 30 and 45 minutes, the reaction will be stopped by the addition of cold acetonitrile solution containing internal standard. The samples taken at all time points were centrifuged at 5000×g for 15 minutes. 50 μL of supernatant are taken into 96-well assay plates pre-added with 50 μL ultrapure water, and then analyzed by LC/MS/MS.

Concentrations of test articles, control compounds in the samples were determined by using LC/MS/MS method. Plotting of the chromatograms and peak area integrations are carried out by Analyst (AB Sciex).

In the determination of the in vitro elimination constant (ke) of the control compounds, the analyte/internal standard peak area ratios will be converted to percentage remaining (% Remaining) with the following equation:

$$\% Remaining = \frac{Peak area ratio of analyte to IS at each time point}{Peak area ratio of analyte to IS at t = 0} \times 100 \%$$

The CLᵢₙₜ of microsomes was calculated using the formula: CL_{int (mic)}=0.693/T_{1/2}/mg microsome protein per mL. The slope was measured by the natural logarithm of the percentage of the residual compound and time, T1/2 was calculated according to the following formula.

$${T}_{1 / 2} = \frac{0.693}{- slope}$$

Exemplary results are summarized in Table 7.

TABLE 7

| | Human LM T½ | Human LM Clint |
|---|---|---|
| Example | (min) | (uL/min/mg) |
| 2 | 25.77 | 53.79 |
| 3 | 239.11 | 5.80 |
| 3A | 223.67 | 6.20 |
| 3B | 259.98 | 5.33 |
| 5 | 27.69 | 50.06 |
| 5A | 24.08 | 57.55 |
| 5B | 29.11 | 47.61 |
| 6 | 48.84 | 28.38 |
| 7 | 49.33 | 28.10 |
| 9 | 255.94 | 5.42 |
| 10 | 308.79 | 4.49 |
| 11 | 485.14 | 2.86 |
| 13 | 54.49 | 25.44 |
| 14 | 35.33 | 39.23 |
| 20 | 35.36 | 39.20 |
| 21 | 84.8 | 16.34 |
| 22 | 149.78 | 9.25 |
| 24 | 333.09 | 4.16 |
| 34 | 2.89 | 479.76 |
| 276* | 290.03 | 6.12 |

### Kinetic Solubility

Aliquots of 8 μL of reference and test compound stock solutions (10 mM/5 mM) are added into 792 μL of 100 mM pH 7.4 phosphate buffer. The final DMSO concentration is 1%. Sample tubes are shaken for 2 hours at 1000 rpm at room temperature. Samples are centrifuged at 12000 rpm for 10 min to precipitate un-dissolved particles. And transfer the supernatants to a new tube or plate. Add 5 μL of samples (no diluted, 10 times diluted and 100 times diluted) and standard curve samples to 95 μL of ACN containing IS for LC-MS/MS analysis. Exemplary results are summarized in Table 8

TABLE 8

| Example | Kinetics Solubility_PBS7.4(μM) |
|---|---|
| 3A | 19.05 |
| 3B | 12.75 |
| 4 | 61.75 |
| 9 | 38.2 |
| 10 | 29 |
| 12 | 7.21 |
| 14 | 16.9 |
| 17 | 11.11 |
| 18 | 41.49 |
| 22 | 19.8 |
| 27 | 44.25 |
| 28 | 20.6 |
| 276* | 12.79 |

Exemplary compounds showed ideal solubility for further development.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A compound having the structural formula (I): 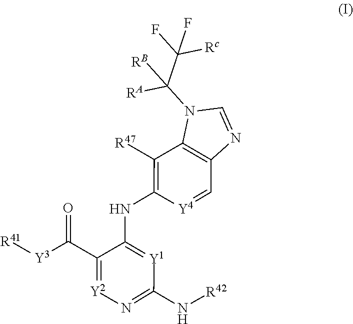
or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
an aryl or heteroaryl group, each substituted with 0-2 R^{42a};
(C═O)R^{42b}; or
(C═O)NHR^{42b};
each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a};
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c};
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a}; and
R⁴⁷ is a C₁₋₃ alkoxy group optionally substituted with 1-3 D or halo.

2. The compound of claim 1, wherein Y⁴ is CH, having the structure:

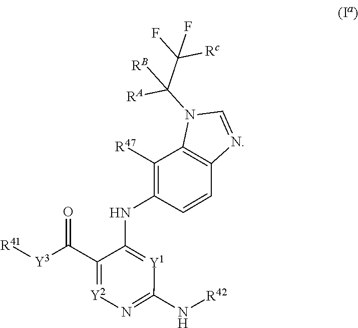

3. The compound of claim 2, wherein Y¹ is CH and Y² is CH, having the structure:

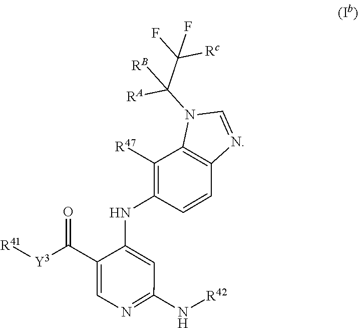

4. The compound of claim 2, wherein Y¹ is N and Y² is CH, having the structure:

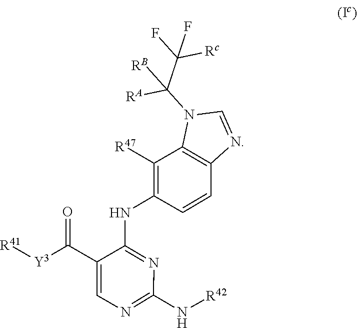

5. The compound of claim 2, wherein Y¹ is CH and Y² is N, having the structure:

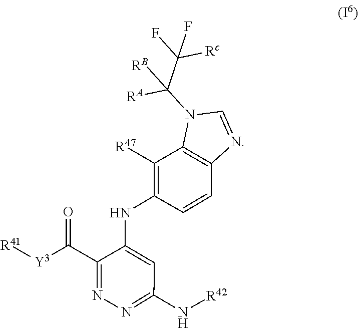

6. The compound of claim 2, wherein Y¹ is N and Y² is N, having the structure:

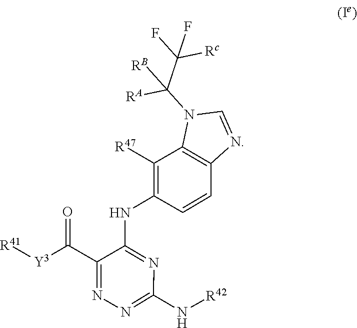

7. The compound of claim 1, wherein Y⁴ is N, having the structure:

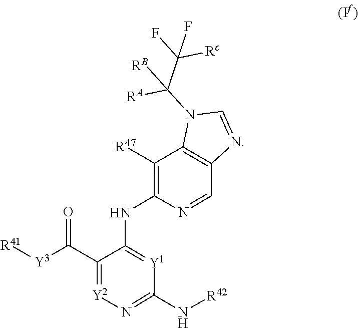

8. The compound of claim 7, wherein Y¹ is CH and Y² is CH, having the structure:

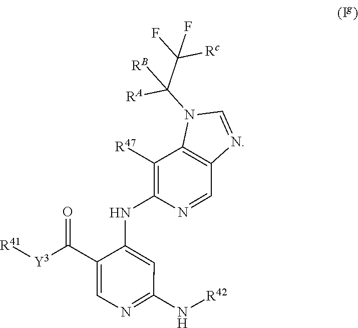

9. The compound of claim 7, wherein Y¹ is N and Y² is CH, having the structure:

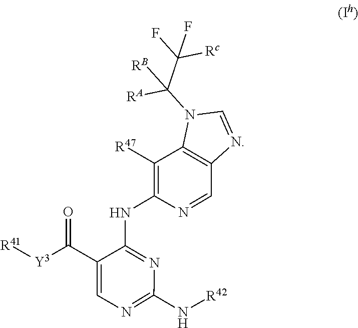

10. The compound of claim 7, wherein Y¹ is CH and Y² is N, having the structure:

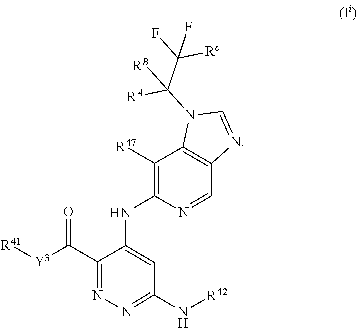

11. The compound of claim 7, wherein Y¹ is N and Y² is N, having the structure:

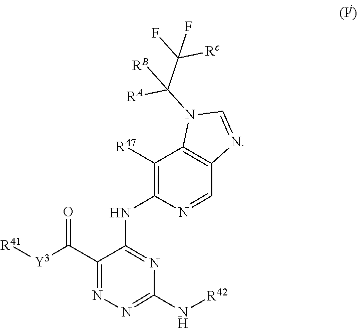

12. The compound of any one of claims 1-11, wherein R^{A} is H and R^{B} is H.

13. The compound of any one of claims 1-12, wherein R^{C} is CH₃.

14. The compound of any one of claims 1-12, wherein R^{C} is not CH₃.

15. A compound having the structural formula (II): 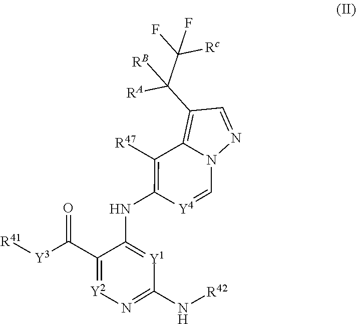
or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
an aryl or heteroaryl group, each substituted with 0-2 R^{42a}; or 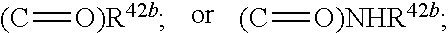
each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a}; and
R⁴⁷ is a C₁₋₃ alkoxy group optionally substituted with 1-3 D or halo.

16. The compound of claim 15, wherein Y⁴ is CH, having the structure:

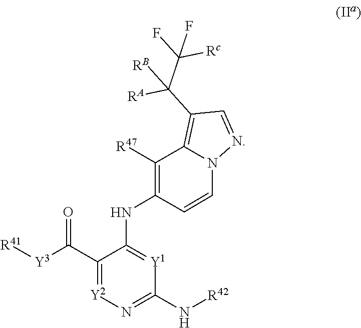

17. The compound of claim 16, wherein Y¹ is CH and Y² is CH, having the structure:

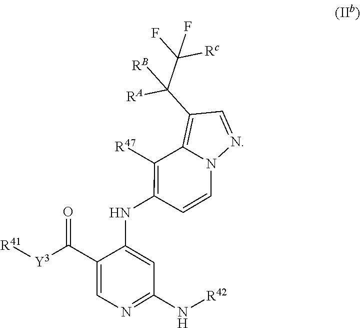

18. The compound of claim 16, wherein Y¹ is CH and Y² is N, having the structure:

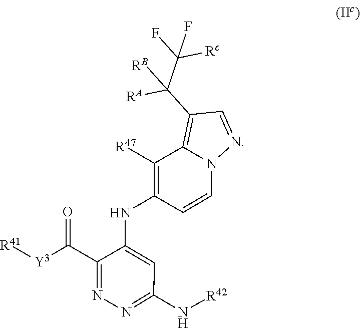

19. The compound of claim 16, wherein Y¹ is N and Y² is CH, having the structure:

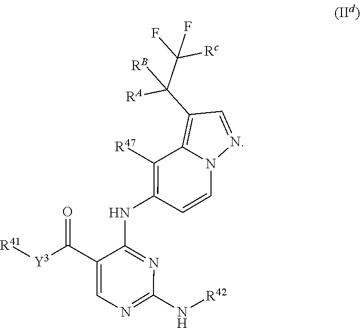

20. The compound of claim 16, wherein Y¹ is N and Y² is N, having the structure:

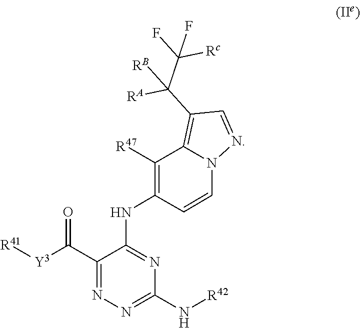

21. The compound of claim 15, wherein Y⁴ is N, having the structure:

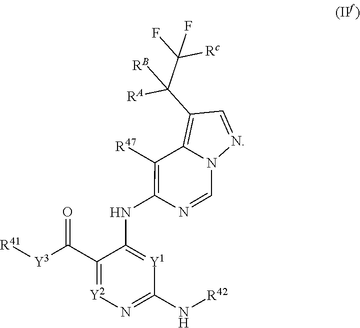

22. The compound of claim 21, wherein Y¹ is CH and Y² is CH, having the structure:

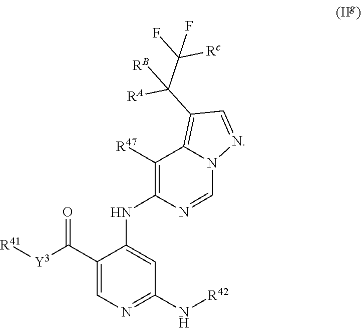

23. The compound of claim 21, wherein Y¹ is N and Y² is CH, having the structure:

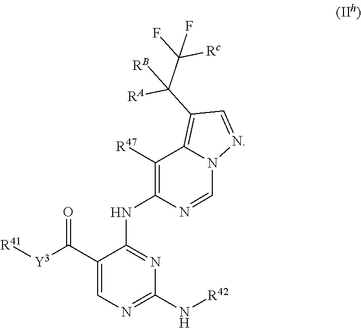

24. The compound of claim 21, wherein Y¹ is CH and Y² is N, having the structure:

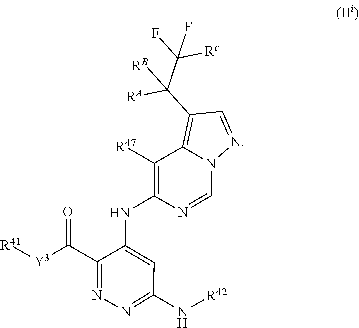

25. The compound of claim 21, wherein Y¹ is N and Y² is N, having the structure:

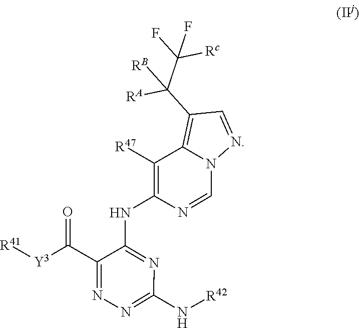

26. The compound of any one of claims 15-25, wherein R^{A} is H and R^{B} is H.

27. The compound of any one of claims 15-26, wherein R^{C} is CH₃.

28. The compound of any one of claims 15-26, wherein R^{C} is not CH₃.

29. A compound having the structural formula (III): 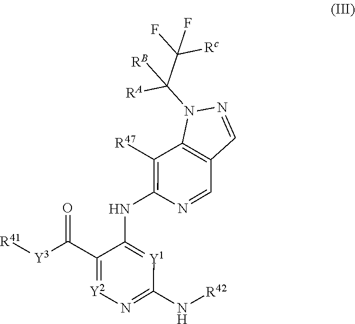
or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
an aryl or heteroaryl group, each substituted with 0-2 R^{42a}; or 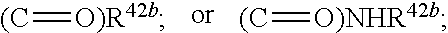
each of R^{A} and R^{B} is independently selected from H, D, halo, OR, and C₁₋₃ alkyl, provided that when both R^{A} and R^{B} is H, R^{C} is not CH₃ or F;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a}; and
R⁴⁷ is a C₁₋₃ alkoxy group optionally substituted with 1-3 D or halo.

30. The compound of claim 29, wherein YI is CH and Y² is CH, having the structure:

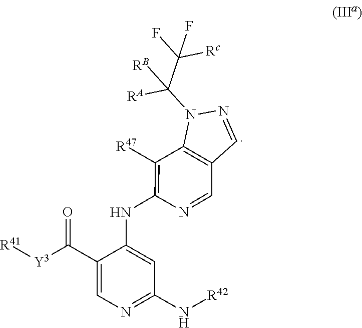

31. The compound of claim 29, wherein YI is CH and Y² is N, having the structure:

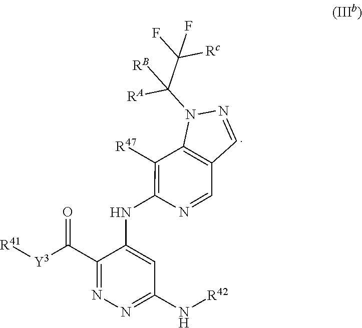

32. The compound of claim 29, wherein Y¹ is N and Y² is CH, having the structure:

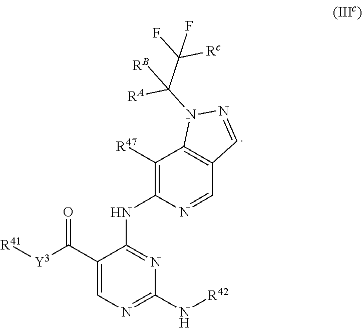

33. The compound of claim 29, wherein Y¹ is N and Y² is N, having the structure:

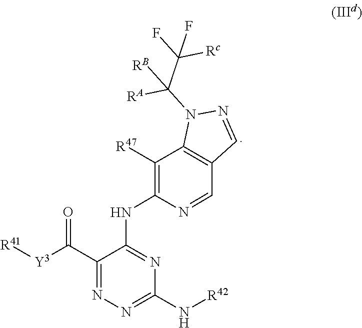

34. The compound of any one of claims 29-33, wherein R^{A} is H and R^{B} is H.

35. The compound of any one of claims 29-33, wherein one of R^{A} and R^{B} is not H.

36. The compound of any one of claims 29-35, wherein R^{C} is not CH₃.

37. A compound having the structural formula (IV): 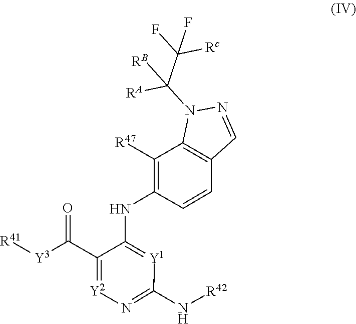
or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
an aryl or heteroaryl group, each substituted with 0-2 R^{42a}; or 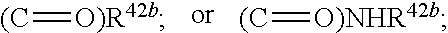
each of R^{A} and R^{B} is independently selected from H, D, halo, OR, and C₁₋₃ alkyl, provided that when both R^{A} and R^{B} is H, R^{C} is not H or F;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a}; and
R⁴⁷ is a C₁₋₃ alkoxy group optionally substituted with 1-3 D or halo.

38. The compound of claim 37, wherein Y¹ is CH and Y² is CH, having the structure:

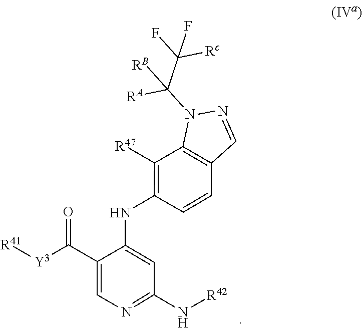

39. The compound of claim 37, wherein YI is N and Y² is CH, having the structure:

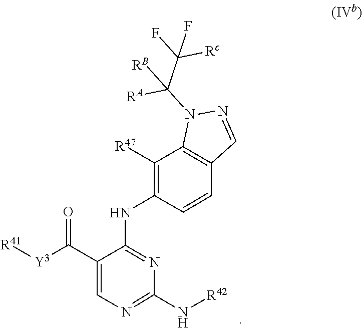

40. The compound of claim 37, wherein Y¹ is CH and Y² is N, having the structure:

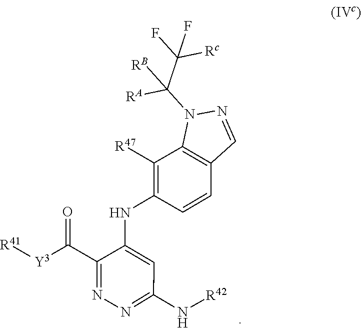

41. The compound of claim 37, wherein Y¹ is N and Y² is N, having the structure:

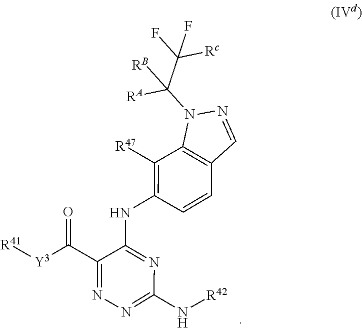

42. The compound of any one of claims 37-41, wherein R^{A} is H and R^{B} is H.

43. The compound of any one of claims 37-41, wherein R^{C} is CH₃.

44. The compound of any one of claims 37-41, wherein R^{C} is not CH₃.

45. A compound having the structural formula (V): 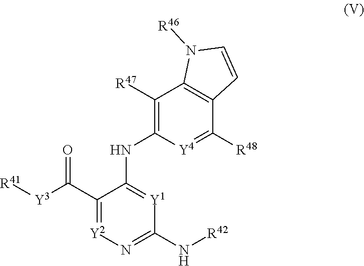
or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
an aryl or heteroaryl group, each substituted with 0-2 R^{42a}; 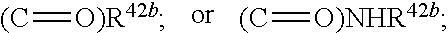
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a};
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a};
R⁴⁶ is H, CD₃, a C₁-C₆ alkyl or C₃-C₆ cycloalkyl, with 0-4 of halogen, CN and OR;
R⁴⁷ is a C₁₋₃ alkoxy group optionally substituted with 1-3 D or halo; and
R⁴⁸ is H, halo or a C₁-C₃ alkyl.

46. The compound of claim 45, wherein Y¹ is CH and Y² is CH, having the structure:

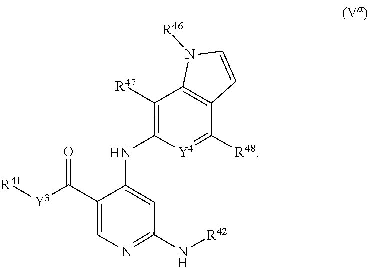

47. The compound of claim 45, wherein Y¹ is N and Y² is CH, having the structure:

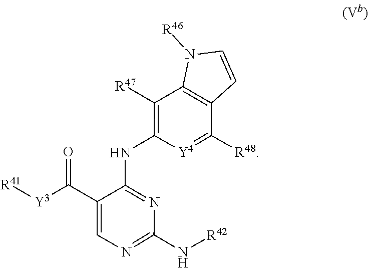

48. The compound of claim 45, wherein Y¹ is CH and Y² is N, having the structure:

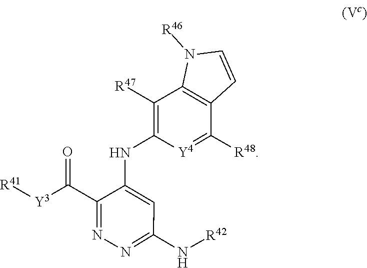

49. The compound of claim 45, wherein Y¹ is N and Y² is N, having the structure:

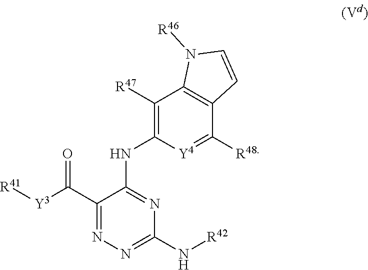

50. The compound of claim 45, having the structure: 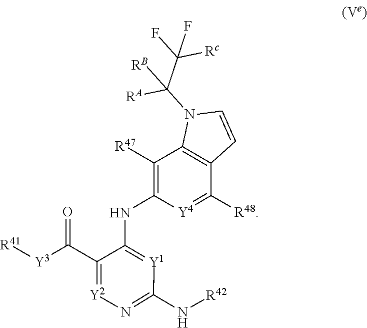
wherein
each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a};
51. The compound of any one of claims 45-50, wherein Y⁴ is CH.

52. The compound of any one of claims 45-50, wherein Y⁴ is N.

53. The compound of any one of claims 45-52, wherein R⁴⁸ is H.

54. The compound of any one of claims 45-52, wherein R⁴⁸ is halo.

55. The compound of any one of claims 1-54, wherein R⁴⁷ is C₁-C₃ alkoxy.

56. The compound of any one of claims 1-54, wherein R⁴⁷ is OCH₃.

57. The compound of any one of claims 1-54, wherein R⁴⁷ is OCD₃.

58. The compound of any one of claims 1-57, wherein Y³ is NR.

59. The compound of claim 58, wherein Y³ is NH.

60. The compound of any one of claims 1-57, wherein Y³ is O.

61. The compound of any one of claims 1-57, wherein Y³ is O—NH.

62. The compound of any one of claims 1-61, wherein R⁴¹ is CH₃.

63. The compound of any one of claims 1-61, wherein R⁴¹ is CD₃.

64. The compound of any one of claims 1-63, wherein R⁴² is R^{42′}.

65. The compound of any one of claims 1-63, wherein R⁴² is (C═O)R^{42b}, wherein R^{42b} is selected from C₁-C₆ alkyl, cyclopropyl or cyclobutyl, substituted with 0-2 R^{42c}.

66. The compound of claim 65, wherein R⁴² is (C═O)R^{42b}, wherein R^{42b} is cyclopropyl, optionally substituted with one or more of F, Cl, CH₃, CF₃ and CN.

67. The compound of claim 65, wherein R⁴² is (C═O)R^{42b}, wherein R^{42b} is bis-spiro-cyclopropyl, optionally substituted with one or more of F, Cl, CH₃, CF₃ and CN.

68. The compound of claim 65, wherein R⁴² is (C═O)R^{42b}, wherein R^{42b} is cyclobutyl, optionally substituted with one or more of F, Cl, CH₃, CF₃ and CN.

69. The compound of claim 65, wherein R⁴² is (C═O)R^{42b}, wherein R^{42b} is C₁-C₆ alkyl, optionally substituted with one or more of F, Cl, CH₃, CF₃, CN, NRR′ and OR.

70. The compound of any one of claims 1-63, wherein R⁴² is (C═O)NHR⁴²6, wherein R^{42b} is selected from C₁-C₆ alkyl, cyclopropyl or cyclobutyl, substituted with 0-2 R^{42c}.

71. The compound of any one of claims 1-63, wherein R⁴² is pyridinyl substituted with 0-2 R^{42c}.

72. The compound of any one of claims 1-63, wherein R⁴² is phenyl substituted with 0-2 R^{42c}.

73. The compound of any one of claims 1-63, wherein R⁴² is pyrazolyl substituted with 0-2 R^{42c}.

74. The compound of any one of claims 1-63, wherein R⁴² is pyrimidyl substituted with 0-2 R^{42c}.

75. The compound of any one of claims 71-74, wherein R^{42c} is CH₃ or CD₃.

76. The compound of any one of claims 71-74, wherein R^{42c} is F.

77. A compound selected from Table 1A or a pharmaceutically acceptable form or an isotope derivative thereof.

78. A compound selected from Table 1B or a pharmaceutically acceptable form or an isotope derivative thereof.

79. A pharmaceutical composition comprising a compound according to any of claims 1-78, effective to treat or reduce one or more diseases or disorders, in a mammal, including a human, and a pharmaceutically acceptable excipient, carrier, or diluent.

80. The pharmaceutical composition of claim 79, being suitable for oral administration.

81. The pharmaceutical composition of claim 79 or 80, being suitable for topical administration.

82. The pharmaceutical composition of any one of claims 79-81, being suitable for GI-restricted administration.

83. The pharmaceutical composition of any one of claims 79-81, being useful to treat or reduce one or more of inflammatory diseases, immune-mediated diseases and cancers, or a related disease or disorder.

84. The pharmaceutical composition of claim 83, wherein the disease or disorder is an inflammatory disease.

85. The pharmaceutical composition of claim 84, wherein the disease or disorder is an immune-mediated disease.

86. The pharmaceutical composition of claim 84, wherein the disease or disorder is cancer.

87. The pharmaceutical composition of claim 86, wherein the disease or disorder is selected from: inflammatory bowel disease, psoriasis, vitiligo, atopic dermatitis, systemic lupus erythematosus, asthma, diabetic nephropathy, chronic myelogenous leukemia (CML), essential thrombocythemia (ET), polycythemia vera (PV), myelofibrosis (MF), breast cancer and ovarian cancer.

88. A unit dosage form comprising a pharmaceutical composition according to any of claims 79-87.

89. The unit dosage form of claim 88, being a tablet.

90. The unit dosage form of claim 88, being a capsule.

91. The unit dosage form of claim 88, being a topical formulation.

92. A method for treating, reducing or preventing a disease or disorder, comprising administering to a subject in need thereof a therapeutically effective amount of a compound having the structural formula of (I): 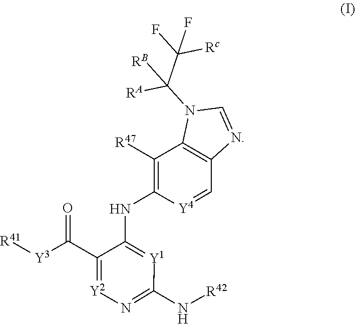
or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
an aryl or heteroaryl group, each substituted with 0-2 R^{42a}; or 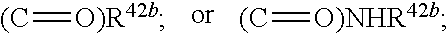
each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a};
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c};
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a}; and
R⁴⁷ is a C₁₋₃ alkoxy group optionally substituted with 1-3 D or halo,
wherein the disease or disorder is selected from inflammatory diseases, immune-mediated diseases, cancer, or a related disease or disorder thereof, in a mammal, including a human.

93. A method for treating, reducing or preventing a disease or disorder, comprising administering to a subject in need thereof a therapeutically effective amount of a compound having the structural formula of (II): 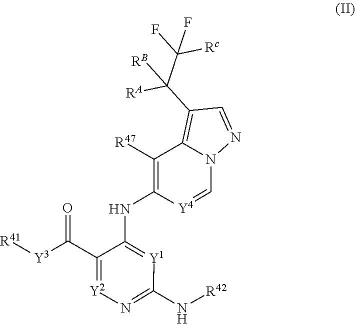
or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
an aryl or heteroaryl group, each substituted with 0-2 R^{42a}; or 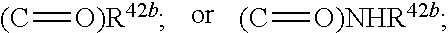
each of R^{A} and R^{B} is independently selected from H, D, halo, OR, and C₁₋₃ alkyl;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a}; and
R⁴⁷ is a C₁₋₃ alkoxy group optionally substituted with 1-3 D or halo,
wherein the disease or disorder is selected from inflammatory diseases, immune-mediated diseases, cancer, or a related disease or disorder thereof, in a mammal, including a human.

94. A method for treating, reducing or preventing a disease or disorder, comprising administering to a subject in need thereof a therapeutically effective amount of a compound having the structural formula of (III): 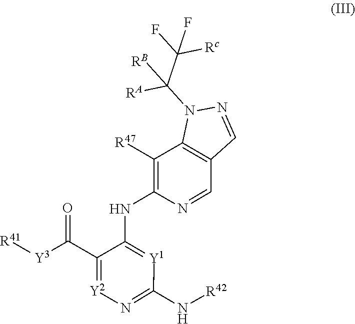
or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
an aryl or heteroaryl group, each substituted with 0-2 R^{42a}; or 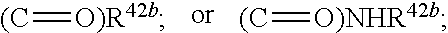
each of R^{A} and R^{B} is independently selected from H, D, halo, OR, and C₁₋₃ alkyl, provided that when both R^{A} and R^{B} is H, R^{C} is not CH₃ or F;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a}; and
R⁴⁷ is a C₁₋₃ alkoxy group optionally substituted with 1-3 D or halo,
wherein the disease or disorder is selected from inflammatory diseases, immune-mediated diseases, cancer, or a related disease or disorder thereof, in a mammal, including a human.

95. A method for treating, reducing or preventing a disease or disorder, comprising administering to a subject in need thereof a therapeutically effective amount of a compound having the structural formula of (IV): 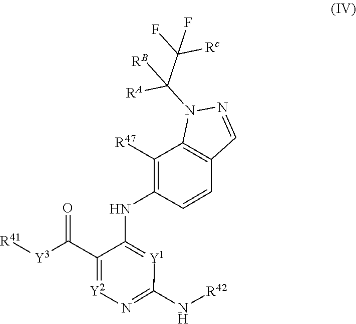
or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
an aryl or heteroaryl group, each substituted with 0-2 R^{42a}; or 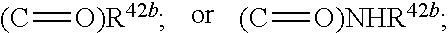
each of R^{A} and R^{B} is independently selected from H, D, halo, OR, and C₁₋₃ alkyl, provided that when both R^{A} and R^{B} is H, R^{C} is not H or F;
R^{C} is H, D, F, Cl, OR, C₁₋₅ alkyl, C₃₋₅ cycloalkyl or heterocyclic group, substituted with 0-2 R^{42a}.
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42a}; and
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a}; and
R⁴⁷ is a C₁₋₃ alkoxy group optionally substituted with 1-3 D or halo,
wherein the disease or disorder is selected from inflammatory diseases, immune-mediated diseases, cancer, or a related disease or disorder thereof, in a mammal, including a human.

96. A method for treating, reducing or preventing a disease or disorder, comprising administering to a subject in need thereof a therapeutically effective amount of a compound having the structural formula of (V): 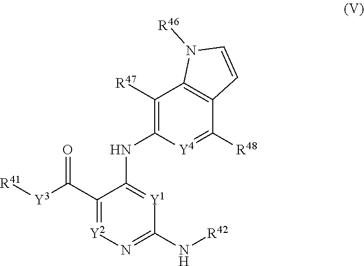
or a pharmaceutically acceptable form or an isotope derivative thereof,
wherein
Y¹ is CH, CF or N;
Y² is CH or N;
Y³ is NR, O, CH₂, CF₂ or O—NH;
Y⁴ is CH or N;
R⁴¹ is a H, F, C₁-C₃ alkyl and CD₃, provided that R⁴¹ is not F when Y³ is NR, O or O—NH;
R⁴² is
R^{42′}, wherein R^{42′} is a C₁-C₆ alkyl, C₃-C₆ cycloalkyl or heterocycloalkyl, each substituted with 0-2 of halogen, CN, OR, NRR′, alkyl, cycloalkyl, heterocyclic, aryl and heteroaryl;
an aryl or heteroaryl group, each substituted with 0-2 R^{42a}; 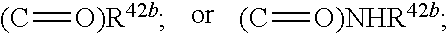
each of R and R′ is independently H or a C₁-C₆ alkyl, C₁-C₆ acyl, or R and R′, together with the nitrogen atom to which they are bound, form a 4- to 7-membered ring comprising 0-2 heteroatoms selected from O, NR, S and SO₂;
R^{42a} at each occurrence is independently D, halo, OH, OR, CH₃, CF₃, CH₂CF₃, CN, C(O)NR, NRR′, (CH₂)ₙNRR′ or a 4- to 6-membered heterocycle having 1-4 heteroatoms selected from N, O and S;
R^{42b} is a C₁₋₆ alkyl or C₃₋₆ cycloalkyl, C₅₋₇ spirocycloalkyl, aryl or heteroaryl, each substituted with 0-2 R^{42c};
R^{42c} at each occurrence is independently halo, CN, OR, NRR′, OCF₃, CF₃, C₁₋₆ alkyl substituted with 0-3 R^{42a}, C₁₋₆ haloalkyl, C₂₋₆ alkenyl substituted with 0-3 R^{42a}, C²⁻⁶ alkynyl substituted with 0-3 R^{42a};
R⁴⁶ is H, CD₃, a C₁-C₆ alkyl or C₃-C₆ cycloalkyl, with 0-4 of halogen, CN and OR;
R⁴⁷ is a C₁₋₃ alkoxy group optionally substituted with 1-3 D or halo; and
R⁴⁸ is H, halo or a C₁-C₃ alkyl,
wherein the disease or disorder is selected from inflammatory diseases, immune-mediated diseases, cancer, or a related disease or disorder thereof, in a mammal, including a human.

97. The method of any one of claims 92-96, wherein the disease or disorder is an inflammatory disease.

98. The method of any one of claims 92-96, wherein the disease or disorder is an immune-mediated disease.

99. The method of any one of claims 92-96, wherein the disease or disorder is cancer.

100. The method of any one of claims 92-99, wherein the disease or disorder is selected from: inflammatory bowel disease, psoriasis, vitiligo, atopic dermatitis, systemic lupus erythematosus, asthma, diabetic nephropathy, chronic myelogenous leukemia (CML), essential thrombocythemia (ET), polycythemia vera (PV), myelofibrosis (MF), breast cancer and ovarian cancer.

101. The method of any of claims 92-100, wherein administration is via oral administration.

102. The method of any of claims 92-100, wherein administration is via topical administration.

103. The method of any of claims 92-100, wherein administration is via GI-restricted administration.

104. Use of a compound of any of claims 1-78, and a pharmaceutically acceptable excipient, carrier, or diluent, in preparation of a medicament for treating a disease or disorder.

105. The use of claim 104, wherein the disease or disorder is one or more of inflammatory diseases, immune-mediated diseases and cancer.

106. The use of claim 105, wherein the disease or disorder is an inflammatory disease.

107. The use of claim 105, wherein the disease or disorder is an immune-mediated disease.

108. The use of claim 105, wherein the disease or disorder is cancer.

109. The use of any one of claims 104-108, wherein the medicament is for oral administration.

110. The use of any one of claims 104-108, wherein the medicament is for topical administration.

111. The use of any one of claims 104-108, wherein the medicament is for GI restriction administration.

112. A method for preparing a compound of any one of claims 1-78.
